SEQUENCE LISTING (8 sequences)

SEQ ID NO: 1 - DNA, 97 bp, Artificial Sequence
  Synthetic polynucleotide
  misc_feature (97)..(97): Modified by phosphorylation
agcttgagatgtgaggaagtagtaatcgctaatgcgtaatcggtaatccgtaatcgctaatgcgtaatcggtaatccgtaatctactccctaacatt

SEQ ID NO: 2 - DNA, 27 bp, Artificial Sequence
  Synthetic polynucleotide
  misc_feature (27)..(27): Modified by biotin
ttcctcacatctcaagctttttttttt

SEQ ID NO: 3 - DNA, 14 bp, Artificial Sequence
  Synthetic polynucleotide
taagttagggagta

SEQ ID NO: 4 - DNA, 41 bp, Artificial Sequence
  Synthetic polynucleotide
  misc_feature (41)..(41): Modified by phosphorylation
agcttgagatgtgaggaagtagtcacctactccctaacatt

SEQ ID NO: 5 - DNA, 57 bp, Artificial Sequence
  Synthetic polynucleotide
  misc_feature (57)..(57): Modified by phosphorylation
agcttgagatgtgaggaagtagtcaccttataaccatactatctactccctaacatt

SEQ ID NO: 6 - DNA, 153 bp, Artificial Sequence
  Synthetic polynucleotide
  misc_feature (153)..(153): Modified by phosphorylation
agcttgagatgtgaggaagtagtaatcggtaaacggtaatccgtaaaccgtaatcgctaaacgctaatgcgtaaagcgtaatcggtaaacggtaatccgtaaaccgtaatcgctaaacgctaatgcgtaaagcgtaatctactccctaacatt

SEQ ID NO: 7 - DNA, 41 bp, Artificial Sequence
  Synthetic polynucleotide
  misc_feature (1)..(1): Modified by biotin
ggccatgctctgtaagttagaaatgctactactattagtag

SEQ ID NO: 8 - DNA, 54 bp, Artificial Sequence
  Synthetic polynucleotide
  misc_feature (1)..(1): Modified by phosphorylation
ctactaatagtagtagcatttctaacttacagagcatggctactccctaactta

(19) United States
(12) Patent Application Publication
(10) Pub. No.: US 2019/0032109 A1
(43) Pub. Date: Jan. 31, 2019

(54) SINGLE MOLECULE TIMERS AND CLOCKS

(71) Applicant: Dana-Farber Cancer Institute, Inc., Boston, MA (US)

(72) Inventors: Alexander E. Johnson-Buck, Brighton, MA (US); William M. Shih, Cambridge, MA (US); Bhavik Nathwani, Boston, MA (US)

(73) Assignee: Dana-Farber Cancer Institute, Inc., Boston, MA (US)

(21) Appl. No.: 16/073,058

(22) PCT Filed: Jan. 26, 2017
(86) PCT No.: PCT/US17/15057

§ 371 Date: Jul. 26, 2018

Related U.S. Application Data
(60) Provisional application No. 62/287,816, filed on Jan. 27, 2016.
(60) Provisional application No. 62/409,305, filed on Oct. 17, 2016.

(51) Int. Cl. C12Q 1/682; C12Q 1/6853; G01N 21/64

(52) U.S. Cl. CPC C12Q 1/682; C12Q 1/6853; G01N 2021/6432; G01N 2021/6441; G01N 21/6428

(57) ABSTRACT

The present disclosure provides, in some aspects, single-molecule timers and clocks, systems and methods for kinetically encoded imaging.

### RELATED APPLICATIONS

This application claims the benefit under 35 U.S.C. § 119(e) of U.S. provisional application No. 62/287,816, filed Jan. 27, 2016, and U.S. provisional application No. 62/409,305, filed Oct. 17, 2016, each of which is incorporated by reference herein it its entirety.

### FEDERALLY SPONSORED RESEARCH

This invention was made with government support under 1435964 awarded by National Science Foundation and under N000141510073 awarded by Office of Naval Research. The government has certain rights in the invention.

### BACKGROUND

Fluorescence microscopy is a technology used for studying a wide range of processes in cell and molecular biology, and is of central importance in both basic and applied biomedical research. This technology has recently been improved by extensions, such as super-resolution microscopy (Huang, B. et al. Annu. Rev. Biochem. 2009, 78 (1), 993), which improves resolution by approximately tenfold compared to a conventional microscope. Nonetheless, only a handful of probes can be distinguished in a visible light microscope due to spectral overlap, thus, it is challenging to image more than a few distinct molecular targets at a time.

### SUMMARY

The present disclosure provides, in some aspects, technology that can reliably distinguish a large number of fluorescent probes used in a single fluorescence microscopy experiment and, by extension, the molecular species represented by the probes. Thus, provided herein, in some embodiments, are imaging methods and associated molecules, referred to as “single-molecule timers” or “single-molecule clocks” or simply “timers” or “clocks,” that provide, for example: (1) control over the duration of probe-target interactions, permitting discrimination among multiple species on the basis of kinetics; (2) rapid multiplexed imaging (e.g., less than an hour) of thousands of distinct targets via kinetic barcoding, which does not require buffer exchange or photobleaching steps, resulting in an acquisition time of minutes rather than hours or days; and (3) compact “molecular barcodes” (e.g., encoded nucleic acids) that do not require spatial encoding of information, permitting, in some aspects, high-density and super-resolution imaging in crowded samples, such as cells and tissues. Advantageously, the methods and molecules of the present disclosure are compatible with standard fluorescence microscopes, thus avoiding the need for custom robotic mixers, microfluidic cells or more specialized instrumentation.

The methods and molecules of the present disclosure use precise single-molecule timing elements to construct compact, high-density labels for fluorescence microscopy. The methods and molecules rely, in part, on engineered quasi-deterministic (exhibiting a variance significantly lower than for a single-step chemical reaction) kinetic properties as well as spectral and spatial information, which permit the production of physically compact barcodes having elements that: (1) are distinguishable from a small number of binding event observations, even when using the same fluorophore; and (2) can be imaged simultaneously in a one-pot mixture of probes. This bypasses time-consuming probe exchange, chemical modification, photobleaching, and ex situ imaging steps, and permits super-resolution imaging of dozens to thousands of targets on a timescale of tens of minutes, for example.

### Single-Molecule Timers

Some aspects of the present disclosure provide a kinetically encoded imaging system, comprising (a) an unpaired initiator nucleic acid comprising a 3′ nucleotide subdomain and a 5′ nucleotide subdomain, (b) a template probe comprising (i) an unpaired 5′ toehold domain, (ii) a hairpin stem domain formed by base pairing between nucleotides located in a 5′ subdomain of the probe and nucleotides located in a 3′ subdomain of the probe, and a hairpin loop domain, and (c) a primer linked to a detectable molecule.

Some aspects of the present disclosure provide a kinetically encoded imaging system, comprising (a) an unpaired initiator nucleic acid comprising a 3′ nucleotide subdomain and a 5′ nucleotide subdomain, (b) a template probe comprising (i) an unpaired 5′ toehold domain, (ii) a hairpin stem domain formed by base pairing between nucleotides located in a 5′ subdomain of the probe and nucleotides located in a 3′ subdomain of the probe, and a hairpin loop domain, wherein the template probe is linked to a detectable molecule, and (c) a primer. In some embodiments, the detectable molecule is linked to the 3′ of the template probe.

Some aspects of the present disclosure provide a kinetically encoded imaging method, comprising: combining in reaction buffer (a) an unpaired initiator nucleic acid comprising a 3′ nucleotide subdomain and a 5′ nucleotide subdomain, wherein the initiator nucleic acid is associated with a target of interest, (b) a hairpin template probe comprising (i) an unpaired 5′ toehold domain, (ii) a hairpin stem domain formed by base pairing between nucleotides located in a 5′ subdomain of the probe and nucleotides located in a 3′ subdomain of the probe, and a hairpin loop domain, (c) a primer linked to a detectable molecule, (d) a DNA polymerase, and (e) dNTPs, thereby forming a reaction mixture; and incubating the reaction mixture under conditions that result in DNA polymerization.

In some embodiments, the 3′ nucleotide subdomain of the initiator of (a) is complementary to and binds to the unpaired toehold domain of the probe of (b), and the 5′ nucleotide subdomain of the initiator of (a) is complementary to and binds to the 5′ subdomain of the probe of (b). In some embodiments, the primer is complementary to and binds to the 3′ subdomain of the probe of (b).

In some embodiments, the method further comprises imaging the reaction mixture during the incubation step and identifying periods of time during which there is an increase in a level of fluorescence relative to a start time control level of fluorescence, thereby identifying dwell times.

In some embodiments, the method further comprises identifying the presence or absence of a target of interest based on the dwell times.

In some embodiments, dNTPs are present at a concentration of 2.5 μM to 10 mM. For example, dNTPs may be present at a concentration of 100 μM.

In some embodiments, the template probe further comprises 3′ phosphate (PO₄²⁻) group.

In some embodiments, the system further comprises a DNA polymerase. In some embodiments, the DNA polymerase has strand displacement activity. For example, the DNA polymerase may be phi29 or Bst DNA polymerase, large fragment.

In some embodiments, the initiator nucleic acid has a length of 15-50 nucleotides. For example, the initiator nucleic acid may have a length of 20-30 nucleotides.

In some embodiments, the 3′ nucleotide subdomain of the initiator nucleic acid has a length of 5-15 nucleotides. In some embodiments, the 5′ nucleotide subdomain of the initiator nucleic acid has a length of 10-20 nucleotides.

In some embodiments, the template probe has a length of 30-200 nucleotides. For example, the template probe may have a length of 30-50 nucleotides.

In some embodiments, the toehold domain has a length of 2-15 nucleotides.

In some embodiments, the hairpin stem domain has a length of 10-20 nucleotides.

In some embodiments, the hairpin loop domain has a length of 4-100 nucleotides. For example, the hairpin loop domain may have a length of 4-20 nucleotides.

In some embodiments, the primer has length of 10-20 nucleotides.

Some aspects of the present disclosure provide a nucleic acid molecule comprising a 5′ paired domain, an internal unpaired domain, and a 3′ paired domain linked to a detectable molecule.

Also provided herein, in some aspects, is a kinetically encoded imaging system, comprising (a) a target nucleic acid, (b) a 5′-phosphorylated nucleic acid probe linked to a 3′ detectable molecule, and (c) a 5′-phosphate-specific exonuclease.

In some embodiments, the probe is complementary to and binds to the target.

Also provided herein, in some aspects, is a kinetically encoded imaging method, comprising: combining in reaction buffer (a) a target nucleic acid, (b) a 5′-phosphorylated nucleic acid probe linked to a 3′ detectable molecule, and (c) a 5′-phosphate-specific exonuclease; and incubating the reaction mixture under conditions that result in exonuclease-mediated degradation of the probe.

Some aspects of the present disclosure provide a kinetically encoded imaging system, comprising: (a) an unpaired initiator nucleic acid; and (b) a first hairpin probe, a second hairpin probe, a third hairpin probe and a fourth hairpin probe, each hairpin probe comprising (i) an unpaired 5′ toehold domain, (ii) a hairpin stem domain formed by intramolecular base pairing between nucleotides located in a 5′ subdomain of the probe and nucleotides located in a 3′ subdomain of the probe, and a hairpin loop domain located between the 5′ subdomain and the 3′ subdomain, wherein the first hairpin probe is linked to a detectable molecule.

In some embodiments, (i) the toehold domain and the 5′ subdomain of the first probe are complementary to and bind to the initiator nucleic acid, (ii) the toehold domain and the 5′ subdomain of the second probe are complementary to and bind to the hairpin stem domain and the 3′ subdomain of the first probe bound to the initiator sequence, (iii) the toehold domain and the 5′ subdomain of the third probe are complementary to and bind to the hairpin stem domain and the 3′ subdomain of the second probe bound to the first probe, and (iv) the toehold domain and the 5′ subdomain of the fourth probe are complementary to and bind to the hairpin stem domain and the 3′ subdomain of the third probe bound to the second probe, and wherein the hairpin loop and 3′ subdomain of the fourth probe are complementary to and bind to the toehold domain and the 5′ subdomain of the first probe. Some aspects of the present disclosure provide a kinetically encoded imaging method, comprising: combining in reaction buffer (a) an unpaired initiator nucleic acid, and (b) a first hairpin probe, a second hairpin probe, a third hairpin probe and a fourth hairpin probe, each hairpin probe comprising (i) an unpaired 5′ toehold domain, (ii) a hairpin stem domain formed by intramolecular base pairing between nucleotides located in a 5′ subdomain of the probe and nucleotides located in a 3′ subdomain of the probe, and a hairpin loop domain located between the 5′ subdomain and the 3′ subdomain, wherein the first hairpin probe is linked to a detectable molecule; and incubating the reaction mixture under conditions that result in DNA hybridization.

### Single-Molecule Clocks

Additionally provided herein, in some embodiments, are compositions, comprising (a) a circular nucleic acid template comprising a primer binding sequence and interlocked with a circular nucleic acid leash, (b) a nucleic acid primer comprising a sequence complementary to the primer binding sequence, and (c) a labeled nucleic acid imager strand. In some embodiments, the compositions further comprise polymerase. In some embodiments, component (c) comprises multiple imager strands, each with different sequences relative to each other and with distinguishable labels. The use of multiple imager strands enables the generation of an ordered series of distinguishable signal ‘pulses’ (e.g., red, red, blue . . . red, red, blue . . . etc.), which provides additional multiplexing capabilities. In some embodiments, the imager strand is bound to a quencher strand that comprises a quencher molecule. In further embodiments, the quencher strand is shorter than the imager strand. In some embodiments, the imager stand is fluorescently labeled on its 3′ end, and the quencher strand comprises a quencher molecule on its 5′ end. In other embodiments, the imager stand is fluorescently labeled on its 5′ end, and the quencher strand comprises a quencher molecule on its 3′ end. In some embodiments, a composition further comprises an endonuclease.

In some embodiments, the present disclosure provides methods that comprise combining in reaction buffer (a) a circular nucleic acid template comprising a primer binding sequence and interlocked with a circular nucleic acid leash, (b) a nucleic acid primer comprising a sequence complementary to the primer binding sequence, (c) a labeled nucleic acid imager strand, (d) a polymerase (e.g., DNA polymerase or RNA polymerase), and (e) deoxyribonucleoside triphosphates (dNTPs) or ribonucleoside triphosphates (NTPs) (depending on whether DNA polymerase or RNA polymerase is used), thereby forming a reaction mixture, and incubating the reaction mixture under conditions that result in nucleic acid polymerization and nucleic acid hybridization.

In some embodiments, the present disclosure further provides compositions, comprising (a) a circular nucleic acid template comprising a primer binding sequence and interlocked with a circular nucleic acid leash, (b) a primer comprising a sequence complementary to the primer binding sequence, and (c) a mixture of nucleoside triphosphates (NTPs or dNTPs) comprising subsets of ATPs (or dATPs), TTPs (or dTTPs), CTPs (or dCTPs) and GTPs (or dGTPs), wherein NTPs of at least one of the subsets comprise a label.

In some embodiments, the present disclosure also provides methods that comprise combining in reaction buffer (a) a circular nucleic acid template comprising a primer binding sequence and interlocked with a circular nucleic acid leash strand, (b) a primer comprising a sequence complementary to the primer binding sequence, (c) a mixture of nucleoside triphosphates (NTPs or dNTPs) comprising subsets of ATPs (or dATPs), TTPs (or dTTPs), CTPs (or dCTPs)and GTPs (or dGTPs), wherein NTPs of at least one of the subsets comprise a label, and (d) a polymerase, thereby forming a reaction mixture, and incubating the reaction mixture under conditions that result in nucleic acid polymerization and nucleic acid hybridization.

### BRIEF DESCRIPTION OF DRAWINGS

FIG. 1 is a schematic depicting a single-molecule timer for kinetically encoded imaging. The graphic shows a polymerase-based timer, which works by introducing a well-defined time delay between the binding of a fluorescent primer (the ON state) and the displacement of the fluorescent primer-template probe complex from the initiator strand by a DNA polymerase (the OFF state). The primary means of controlling the characteristic lifetime, or “mean dwell time,” of a timer probe is by changing the length of the hairpin loop domain of the template, as the “clock” graphic indicates.

FIG. 2 (left panel) shows that the equilibrium two-state binding of a conventional probe (as in DNA-PAINT) results in a wide range of bound-state dwell times that follow an exponential distribution whose standard deviation (σ) is equal to the mean (μ) of the distribution. FIG. 2 (right panel) shows a single-molecule timer exploiting a series of irreversible chemical steps that must occur between binding and dissociation of the probe, resulting in a narrower gamma distribution of dwell times and a standard deviation reduced in proportion to √N, where N is the number of irreversible steps.

FIG. 3A shows an implementation of single-molecule timers using a DNA polymerase (DNAP) with strand displacement activity. FIG. 3B depicts a representative single-molecule fluorescence trajectory showing four consecutive timer binding cycles in the same location on a coverslip using a 41-nucleotide template.

FIGS. 4A-4D show design schematics (left panels) and single-molecule fluorescence traces (right panels) of four different single-molecule timer templates of varying loop lengths: 41 nucleotides (nt) (FIG. 4A), 57 nt (FIG. 4B), 97 nt (FIG. 4C), and 153 nt (FIG. 4D). FIG. 4E illustrates the dwell time distributions and gamma probability distribution fits for the four timer templates shown in FIGS. 4A-4D. n=143, 154, 196, and 155 binding events for the 41 nt, 57 nt, 97 nt, and 153 nt templates, respectively. FIG. 4F depicts the dependence of mean dwell time on total template length. See Example 1.

FIGS. 5A-5B show parameters for gamma fit versus template length. See Example 2.

FIGS. 6A-6B show variation of the dwell time of a single-molecule timer by adjusting the concentration of deoxyribonucleoside triphosphates (dNTPs). See Example 3.

FIGS. 7A-7B show examples of kinetic barcoding with single-molecule timers (also referred to in the figures as “clocks”). Multiple timers can be used in the same experiment to construct barcodes for multiplexed imaging. FIG. 7A depicts a 3-bit barcoding scheme that utilizes a DNA-PAINT probe with exponential dissociation kinetics, and two single-molecule timers with gamma-distributed kinetics. FIG. 7B depicts a 4-bit barcode scheme. See Example 4.

FIGS. 8A-8B show an 8-base pair PAINT strand as an additional “bit” for barcoding. See Example 5.

FIGS. 9A-9B show graphs representative of the simultaneous use of multiple timers. See Example 6.

FIGS. 10A-10C show dwell time distributions and exponential fits for binding events lasting longer than 30 seconds. See Example 7.

FIGS. 11A-11B depict examples of exonuclease-based timers, which use degradative enzymes rather than polymerases.

FIG. 12 is a schematic depicting multiplexed super-resolution microscopy of cells and tissues using single-molecule timers.

FIGS. 13A-13E provide another example of a polymerase-based single-molecule timer. See Example 8.

FIG. 14 shows an implementation of single-molecule timers using a DNA hybridization cascade.

FIG. 15 shows a fluorescence scan of a native polyacrylamide gel electrophoresis experiment demonstrating that the four-way junction (waste) complex forms upon the addition of all four hairpin template probes and the initiator (target) nucleic acid.

FIG. 16 shows a fluorescence scan of a native polyacrylamide gel electrophoresis experiment in which the formation of complexes from the hairpin template probes is monitored over time.

FIG. 17 shows data from internal reflection fluorescence (TIRF) microscopy at varying template probe concentrations.

FIG. 18 shows a histogram of dwell times in the fluorescent state for a 4-step hybridization cascade along with a gamma distribution for a process with 4 steps (shape parameter=4) with a mean lifetime of 2 seconds per step.

FIG. 19 (left panel) shows that the equilibrium 1-step binding of a conventional probe (as in DNA-PAINT) results in a wide range of bound-state dwell times that follow an exponential distribution whose standard deviation (σ) is equal to the mean (μ) of the distribution. FIG. 19 (right panel) shows a single-molecule clock exploiting a series of irreversible chemical steps that must occur between binding and dissociation of the probe, resulting in a narrower gamma distribution of dwell times and a standard deviation reduced in proportion to √N, where N is the number of irreversible steps. The same principle is extended to delay times between any two signaling events (appearance or disappearance of a signal) in single-molecule clocks.

FIG. 20 shows an example of periodic signal generation using a circular template, which permits control of the delay between any two signaling events of interest. The polymerase remains bound through multiple cycles for a rapid readout. Each cycle maybe a single time delay (Δt) or an ordered set of time delays (Δt₁, Δt₂, Δt₃. . . ). Identity can be encoded in order of (multicolor) probe binding as well as timing of events. Circular permutations (e.g., Red, Blue, Blue (RBB), BRB, BBR) may be distinguishable if a suitable pause period is inserted to signal the start of a new cycle. Advantageously, single-molecule clock provide control of both the dwell time of the fluorescent (probe-bound) state as well as (or alternatively) of the non-fluorescent state, or the interval between consecutive binding events or consecutive dissociation events.

FIG. 21 shows examples of molecular barcodes resembling a multicolor Morse code.

FIGS. 22A-22B show examples of graphic simulations of periodic single-molecule clocks using a circular template.

FIGS. 23A-23B show graphs depicting periodic fluorescent pulses.

FIG. 24 shows graphs demonstrating that periodic signaling events, in general, come in bursts separated by longer wait times.

FIG. 25 shows a graph demonstrating that the overall distribution may be the results of a fundamental frequency and ‘undertones’ (gaps from missed events).

FIG. 26 shows a schematic of an example of multiplexed super-resolution microscopy.

FIG. 27 shows a schematic of an example of a Morse probe.

FIG. 28A shows an illustrative example of an imager-quencher pair. Fluorophore functionalization of the imager strand occurs at its 3′ end. In this example, the system uses an 8 nucleotide toehold. The length of the toehold, however, may be longer than 8 nucleotides. For example, the toehold may have a length of 5-20 nucleotides, 5-10 nucleotide, or 8-10 nucleotides. FIG. 28B is a graph that illustrates that a 1:4 (imager:quencher) ratio results in minimal background during Total Internal Reflection Fluorescence (TIRF) imaging.

FIG. 29A shows TIRF measurements of repeated fluorescent pulses generated by repeating binding and displacement of the imager strand to the template (gray line). The fit used to estimate the arrival time is shown in black. FIG. 29B demonstrates that the arrival times fit a gamma distribution.

### DESCRIPTION

The ability to rapidly image a large fraction of the proteome and transcriptome of intact cells and tissues (e.g., from a tumor biopsy) at super-resolution yields critical insights for the personalized diagnosis and treatment of cancer and other diseases. In addition to information about tumor heterogeneity and drug resistance, spatially resolved proteomics reveals nontrivial subcellular organization of surface antigens that can guide the development of more effective targeted therapies, especially those involving multivalent recognition, for example. The limited capacity of light microscopy for multiplexing (3-4 colors) limits the number of targets that can be simultaneously imaged. Provided herein are compact barcodes that encode information in the time domain as a series of deterministic fluorescence pulses, akin to a “molecular Morse code” with high information content. These compact barcodes, referred to as “single-molecule timers,” may be used to achieve temporal encoding by coupling the dissociation of a fluorescent probe to a series of irreversible reactions. “Single-molecule timers” and “single-molecule clocks” permit one to readily distinguish among multiple fluorescent probes based on the temporal pattern of fluorescence intensity, even when they are not separable by color or position. The timers and clocks permits the spatially resolved detection of thousands of distinct molecular targets in a single imaging experiment lasting only ˜10 minutes, for example. Following validation in well-studied mammalian cell lines, these kinetic barcodes may be used to rapidly profile RNA and/or protein expression within intact cancer cells and tissue samples, with single-molecule sensitivity and super-resolution, thus guiding the development of more effective personalized treatments for cancer and other diseases.

### Single-Molecule Timers

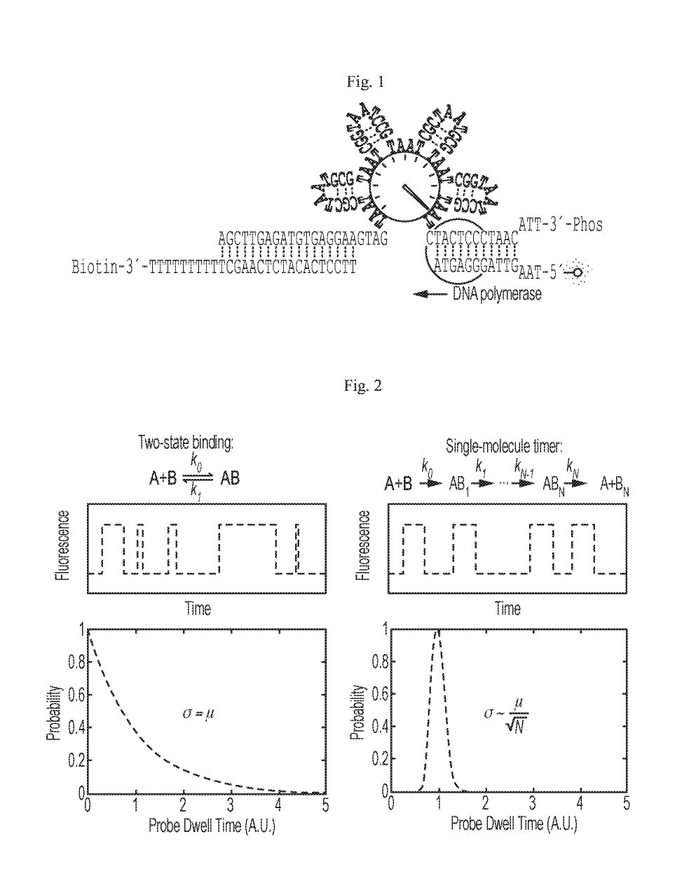

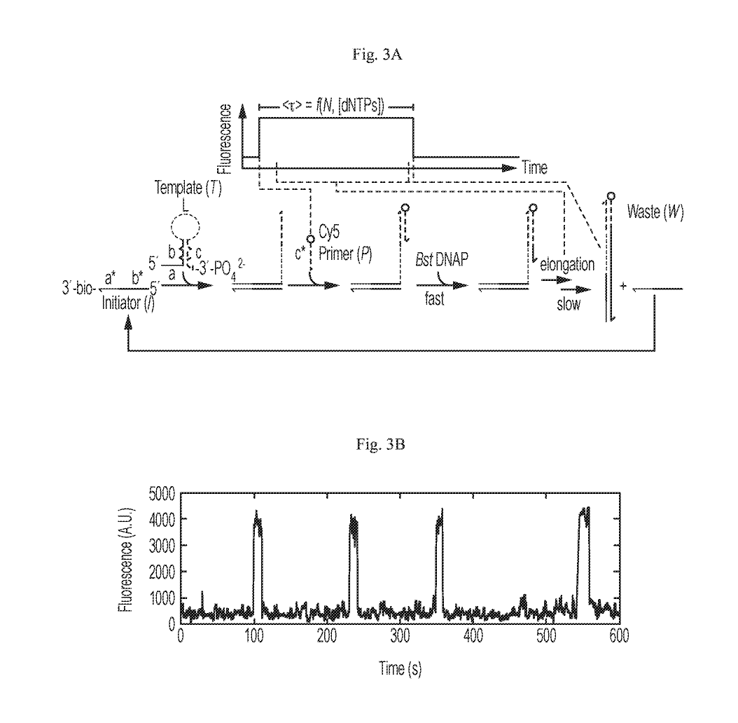

Single-molecule timers, as provided herein, in some aspects, utilize a cascade of several irreversible reactions to establish a well-defined time delay between binding of a fluorescent primer (ON State) to a target and subsequent displacement of the fluorescent primer (in the form of an elongated waste complex, as described below) from the target by a DNA polymerase (OFF state) (see, e.g., FIGS. 1 and 3A); the summation of several exponentially distributed variables yields a sharp gamma distribution of dwell times (see, e.g., FIG. 2 (right panel)). That is, each binding event has a precisely determined duration, with a degree of randomness that decreases in proportion to

$$\frac{1}{\sqrt{N}} ,$$

where N is the number or irreversible reactions between binding and dissociation. This quasi-deterministic behavior permits the unambiguous identification of a target by observing the duration of a single binding event.

To provide robust timer behavior, the lifetime of fluorescent primer binding to the target is controlled primarily by the series of irreversible reactions—this irreversible cascade is rate-limiting. The reactions may also have partly reversible character as long as there is a forward bias in the equilibrium governing each step; in such cases, the degree of randomness (or width relative to the mean value) of the dwell time distribution increases with increasing reversibility. The present disclosure provides polymerase-based single-molecule timers, exonuclease-based single-molecule timers, and single-molecule timers constructed from hybridization cascades. For polymerase-based single-molecule timers, the polymerase concentration may be kept high and/or the polymerization rate may kept low. The polymerization rate may be kept low by modifying, for example, buffer conditions, temperature, and dNTP concentrations. For exonuclease-based single-molecule timers, the exonuclease concentration may be kept high and/or the degradation rate may be kept low.

### Principles of Single-Molecule Timers

The binding equilibrium of a bimolecular complex can usually be approximated as a two-state system characterized by a bimolecular association rate constant k₀ and a unimolecular dissociation rate constant k₁ (FIG. 2, left panel). Thus, when the reversible binding of a fluorescent probe to an immobilized target is monitored at the single-molecule level, the dwell times in the high-fluorescence (bound) state are exponentially distributed, with a standard deviation equal to the mean dwell time μ (FIG. 2, left panel). Effectively, the complex has no memory of how long it has existed, and its probability of dissociating within an interval of time Δt is independent of its history, resulting in a broad range of complex lifetimes. In contrast, single-molecule timers pass through a series of intermediates via irreversible reactions characterized by rate constants k₁, k₂, . . . k_{N} before dissociation can occur (FIG. 2, right panel). While the lifetime of each intermediate is exponentially distributed, the sum

$${τ}_{N} = {\sum }_{i = 1}^{N} \frac{1}{{k}_{i}} \qquad ( 1 )$$

obeys a gamma distribution, with standard deviation proportional to 1/\sqrt{N} (FIG. 2, right panel). That is, as the number of rate-limiting irreversible steps increases, the lifetime of the complex becomes more narrowly distributed about its expectation value, or more deterministic.

Although the single-molecule timer principle may be implemented in a number of different ways, one approach is to use a processive enzyme such as a DNA polymerase (DNAP). Described herein is a system in which a DNAP with strand displacement activity controls the delay between the binding and dissociation of a fluorescent probe (FIG. 3A). In this system, a hairpin template T binds to an initiator strand I, resulting in the opening of the hairpin and exposure of a primer binding site on T. Subsequently, a fluorophore-labeled primer P binds to the complex, resulting in an increase in localized fluorescence. Almost immediately, the DNAP binds and begins elongating the primer. Upon reaching the end of the template, the strand displacement activity of the DNAP causes the fluorescent waste complex W to dissociate from I, resulting in a loss of fluorescence from the binding site. Importantly, the concentration of the DNAP is high enough (>1 μM) to ensure that its binding is rapid relative to elongation. The dwell time in the high-fluorescence state is thus controlled by the rate of nucleotide addition, and is expected to exhibit a gamma distribution of dwell times whose shape and mean value is dependent on the length of the template as well as the concentration of dNTPs.

### Polymerase-Based Single-Molecule Timers

With polymerase-based single-molecule timers, extension of a DNA primer by a polymerase supplies a series of irreversible reactions that constitute the timer (see, e.g., FIG. 3A). A specific target sequence of DNA (“Initiator (I),” which may be associated with an imaging target of interest, such as a cellular protein or RNA molecule) induces the opening of a hairpin template probe (“Template (T)”), exposing a binding site for a primer. A fluorescently labeled primer (“Primer (P)”) then binds to the binding site, resulting in a detectable increase in fluorescence at the site of binding. A DNA polymerase with strand displacement activity (e.g., Bst DNA polymerase) then binds to the template probe and begins extending the primer. Upon reaching the end of the template, the strand displacement activity of the DNAP causes the fluorescent primer-template probe complex (“Waste complex (W)”) to dissociate from I, resulting in a loss of fluorescence from the binding site. The target can subsequently bind another copy of template probe, thus initiating another cycle of polymerization and dissociation. The concentration of the DNAP is high enough (e.g., >1 μM) to ensure that its binding is rapid relative to elongation. The dwell time in the high-fluorescence state, in this example, is thus controlled by the rate of nucleotide addition, and exhibits a gamma distribution of dwell times having shape and mean value that are dependent on the length of the template as well as the concentration of dNTPs.

An “initiator (I)” refers to a contiguous sequence of nucleotides to which a template probe binds (hybridize). An initiator may form part of the sequence of a nucleic acid (e.g., DNA or RNA) target of interest, or an initiator may be an independent molecule associated with (e.g., directly or indirectly linked to) a target of interest (e.g., a protein or other biomolecule). For example, an initiator may be an oligonucleotide linked to protein (or other biomolecule) of interest. An initiator (see FIG. 3A as an illustrative example) includes two subdomains: a 5′ subdomain (“b*”) that binds to a hairpin stem domain (“b”) of a template probe; and a 3′ subdomain (“a*”), adjacent to (directly adjacent to) the 5′ subdomain (“b”), that binds to a 5′ single-stranded domain of the template probe. The length of an initiator may vary and depends, in part, on the length of the template probe, particularly the hairpin domain of the template probe. In some embodiments, an initiator has a length of 10-50 nucleotides. For example, an initiator may have a length of 10-45, 10-40, 10-35, 10-30, 10-25, 10-20, 10-15, 15-50, 15-45, 15-40, 15-35, 15-30, 15-25, 15-20, 20-50, 20-45, 20-40, 20-35, 20-30, 20-25, 25-50, 25-45, 25-40, 25-35, 25-30, 30-50, 30-45, 30-40, 30-35, 35-50, 35-45, 35-40, 40-50, 40-45 or 45-50 nucleotides. In some embodiments, an initiator has a length of 10, 15, 20, 25, 30, 35, 40, 45 or 50 nucleotides. In some embodiments, an initiator has a length of 15, 16, 17, 18, 19, 20, 21, 22, 23, 24 or 25 nucleotides. An initiator, in some embodiments, is longer than 50 nucleotides, or shorter than 10 nucleotides.

In some embodiments, a target of interest is associated with more than one initiator. For example, more than one template probe may bind to the same target. Thus, in some embodiments, a target of interest includes, or is linked to an oligonucleotide that includes, a series of initiators (each including a 3′ and 5′ subdomain) such that multiple different template probes are capable of binding to a single target of interest. In some embodiments, a target of interest is associated with 2-50 different single-molecule timers. For example, a target of interest may be associated with 2-5, 2-10, 2-20, 2-15, 2-20, 2-25, 2-30, 2-35, 2-40, 2-45, 2-50, 5-10, 5-15, 5-20, 5-25, 5-30, 5-35, 5-40, 5-45, 5-50, 10-15, 10-20, 10-25, 10-30, 10-35, 10-40, 10-45 or 10-50 different single-molecule timers. In some embodiments, a target of interest is associated with 1, 2, 3, 4, 5, 6, 7, 8, 9, 10, 11, 12, 13, 14 or 15 different single-molecule timers.

The length of each subdomain of an initiator may vary and depends, in part, on the lengths of the domains of a corresponding template probe to which the initiator binds. In particular, the length of the 5′ subdomain of the initiator typically depends on the length of the hairpin stem domain of the template probe, while the 3′ subdomain of the initiator typically depends on the length of the 5′ single-stranded domain of the template probe.

In some embodiments, a 5′ subdomain of an initiator has a length of 5-40 nucleotides. For example, a 5′ subdomain of an initiator may have a length of 5-35, 5-30, 5-25, 5-20, 5-15, 5-10, 10-40, 10-35, 10-30, 10-25, 10-20, 10-15, 15-40, 15-35, 15-30, 15-25, 15-20, 20-40, 20-35, 20-30, 20-25, 25-40, 25-35, 25-30, 30-40, 30-35 or 35-40 nucleotides. In some embodiments, a 5′ subdomain of an initiator has a length of 5, 10, 15, 20, 25, 30, 35 or 40 nucleotides. In some embodiments, a 5′ subdomain of an initiator has a length of 10, 11, 12, 13, 14, 15, 16, 17, 18, 19, 20, 21, 22, 23, 24 or 25 nucleotides. A 5′ subdomain of an initiator, in some embodiments, is longer than 40 nucleotides, or shorter than 5 nucleotides.

In some embodiments, a 3′ subdomain of an initiator has a length of 5-40 nucleotides. For example, a 3′ subdomain of an initiator may have a length of 5-35, 5-30, 5-25, 5-20, 5-15, 5-10, 10-40, 10-35, 10-30, 10-25, 10-20, 10-15, 15-40, 15-35, 15-30, 15-25, 15-20, 20-40, 20-35, 20-30, 20-25, 25-40, 25-35, 25-30, 30-40, 30-35 or 35-40 nucleotides. In some embodiments, a 3′ subdomain of an initiator has a length of 5, 10, 15, 20, 25, 30, 35 or 40 nucleotides. In some embodiments, a 3′ subdomain of an initiator has a length of 5, 6, 7, 8, 9, 10, 11, 12, 13, 14, 15, 16, 17, 18, 19, 20, 21, 22, 23, 24 or 25 nucleotides. A 5′ subdomain of an initiator, in some embodiments, is longer than 40 nucleotides, or shorter than 5 nucleotides.

A “template probe (T)” refers to a nucleic acid hairpin molecule that binds to an initiator and a fluorescent primer. For simplicity, the domains of a template probe are described in the context of a single strand of nucleic acid. For example, a nucleic acid hairpin molecule occurs when two domains of the same strand, usually complementary in nucleotide sequence when read in opposite directions, base-pair to form a double helix (hairpin stem) that ends in an unpaired loop (hairpin loop). It should be understood, however, that a single strand of nucleic acid may be made up of a contiguous sequence of nucleotides, or a single strand of nucleic acid may be made up of two or more domains of contiguous sequences of nucleotides, each domain joined by a linker (e.g., nucleic acid or chemical linker).

A template probe (see FIG. 3A as an illustrative example) includes a 5′ toehold domain (“a”) linked to a hairpin stem domain (e.g., formed by intramolecular binding of subdomain “b” to subdomain “c”) linked to a hairpin loop domain (“L). Typically, a template probe also includes a 3′ phosphate (PO₄²⁻) group to block the addition of nucleotides to its 3′ end by a DNA polymerase. The length of a template probe may vary. In some embodiments, a template probe has a length of 25-300 nucleotides. For example, a template probe may have a length of 25-250, 25-200, 25-150, 25-100, 25-50, 50-300, 50-250, 50-200, 50-150 or 50-100 nucleotides. In some embodiments, a template probe has a length of 30-50, 40-60, 50-70, 60-80, 70-90, 80-100, 100-125, 100-150 or 100-200 nucleotides. In some embodiments, a template probe has a length of 35, 36, 37, 38, 39, 40, 41, 42, 43, 44, 45, 46, 47, 48, 49. 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 nucleotides. A template probe, in some embodiments, is longer than 300 nucleotides, or shorter than 25 nucleotides.

A “toehold domain” refers to an unpaired sequence of nucleotides located at the 5′ end of the template probe and is complementary to (and binds to) the 3′ subdomain of an initiator. The length of a toehold domain may vary. In some embodiments, a toehold domain has a length of 5-40 nucleotides. For example, a toehold domain may have a length of 2-35, 2-30, 2-25, 2-20, 2-15, 2-10, 5-35, 5-30, 5-25, 5-20, 5-15, 5-10, 10-40, 10-35, 10-30, 10-25, 10-20, 10-15, 15-40, 15-35, 15-30, 15-25, 15-20, 20-40, 20-35, 20-30, 20-25, 25-40, 25-35, 25-30, 30-40, 30-35 or 35-40 nucleotides. In some embodiments, a toehold domain has a length of 5, 10, 15, 20, 25, 30, 35 or 40 nucleotides. In some embodiments, a toehold domain has a length of 10, 11, 12, 13, 14, 15, 16, 17, 18, 19, 20, 21, 22, 23, 24 or 25 nucleotides. A toehold domain, in some embodiments, is longer than 40 nucleotides, or shorter than 5 nucleotides.

A 5′ single-stranded toehold domain of a template probe binds to an initiator via a toehold-mediated strand displacement reaction (Zhang D. & Winfree E. JACS 2009, 131(47, 17303-17314; Zhang D. & Seelig G Nature Chemistry 2011, 3, 103-113). In this reaction (see FIG. 3A as an illustrative example), the initiator (I) binds to the single-stranded toehold domain of a template probe (“a”) and displaces one of the subdomains (“c”) of the double-stranded hairpin stem domain of the template probe through a branch migration process. The overall effect is that one of the subdomains (“c”) of the hairpin stem domain is replaced with the initiator.

A “hairpin stem domain” refers to a paired sequence of nucleotides (e.g., Watson-Crick nucleobase pairing) located adjacent to (and 3′ from) the unpaired toehold domain of a template probe. The hairpin stem domain is formed by intramolecular base pairing of two subdomains of a template probe: e.g., an internal subdomain located 3′ from and adjacent to the toehold domain bound (hybridized) to a subdomain located at the 3′ end of the template probe. The length of a hairpin stem domain may vary. In some embodiments, a hairpin stem domain has a length of 5-40 nucleotides. For example, a hairpin stem domain may have a length of 5-35, 5-30, 5-25, 5-20, 5-15, 5-10, 10-40, 10-35, 10-30, 10-25, 10-20, 10-15, 15-40, 15-35, 15-30, 15-25, 15-20, 20-40, 20-35, 20-30, 20-25, 25-40, 25-35, 25-30, 30-40, 30-35 or 35-40 nucleotides. In some embodiments, a hairpin stem domain has a length of 5, 10, 15, 20, 25, 30, 35 or 40 nucleotides. In some embodiments, a hairpin stem domain has a length of 10, 11, 12, 13, 14, 15, 16, 17, 18, 19, 20, 21, 22, 23, 24 or 25 nucleotides. A hairpin stem domain, in some embodiments, is longer than 40 nucleotides, or shorter than 5 nucleotides.

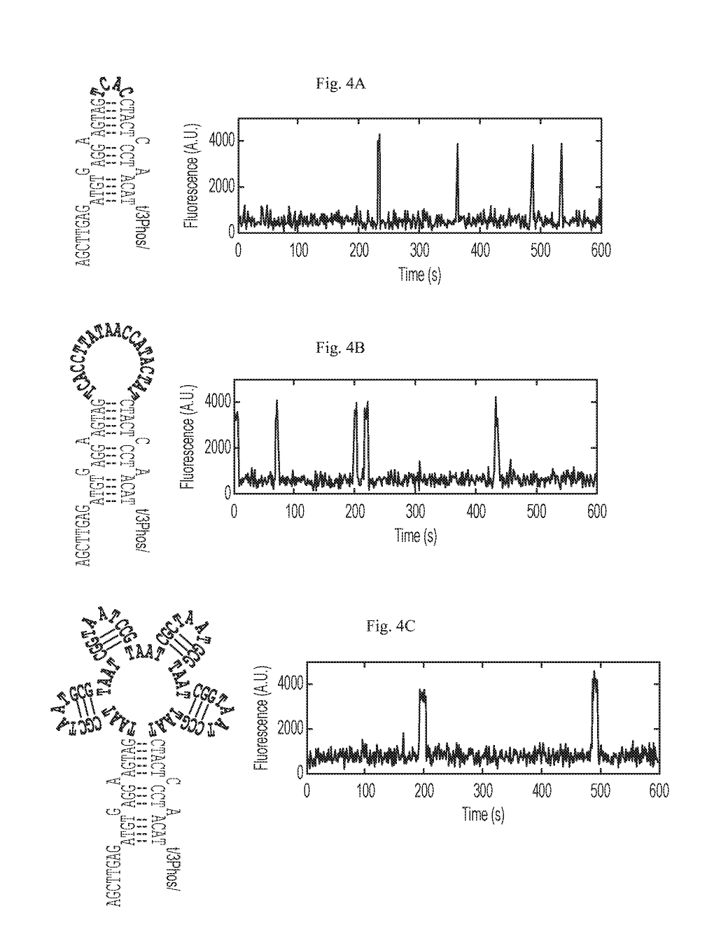

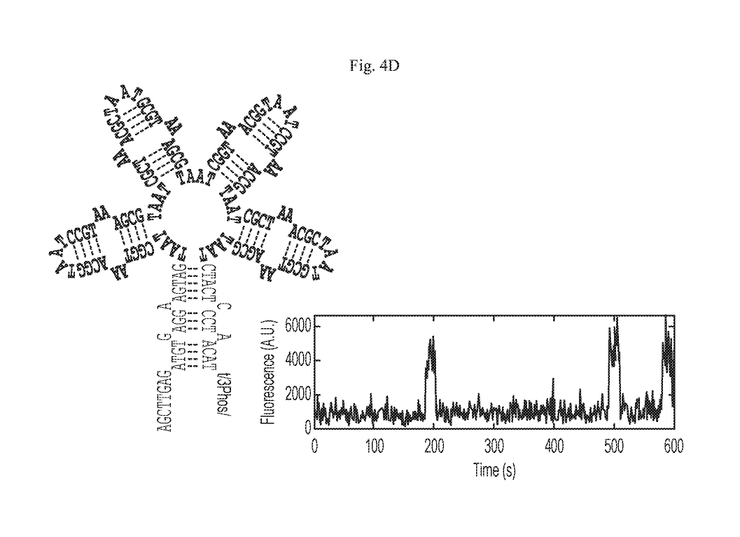

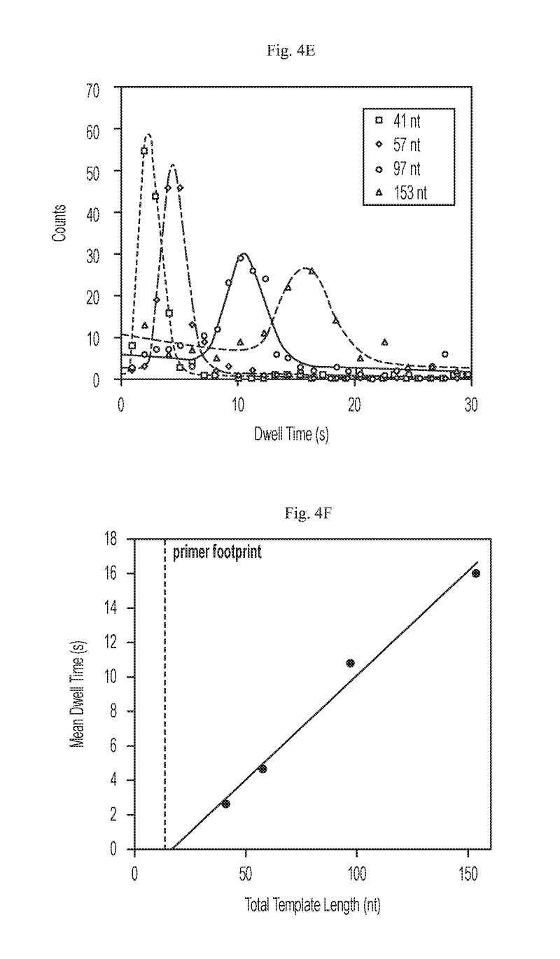

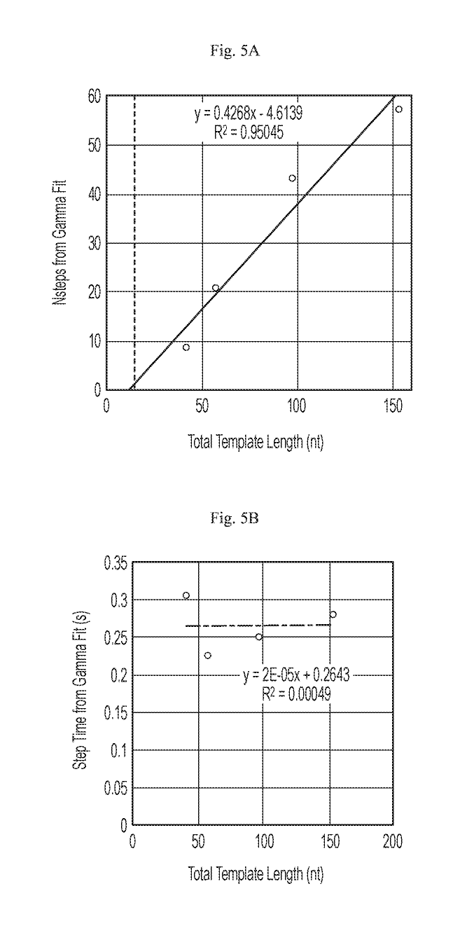

While a hairpin stem domain is generally formed by intramolecular base pairing of two subdomains of a template probe, it should be understood that this paired domain may contain at least one mismatch pair (e.g., pairing of A with C or G, or pairing of T with C or G), as shown in the example template probes depicted in FIGS. 4A-4C, 6A and 10A-10C. Mismatch base pairs may be introduced to permit binding between sequences of the 5′ subdomain of the hairpin stem domain and the 5′ subdomain of the initiator (see, e.g., FIG. 3A, b/b*) and between sequences of the 3′ subdomain of the hairpin stem domain and the primer (see, e.g., FIG. 3A, c/c*) while preventing direct binding between the 5′ subdomain of the initiator and the primer (see, e.g., FIG. 3A, b*/c*). That is, the mismatch base pairs enable the formation of a hairpin template structure to which the initiator and primer may bind, while precluding/reducing direct interactions between the initiator and primer. The number of mismatches in hairpin stem domain may depend on the nucleotide composition and length of the domain. In some embodiments, the hairpin stem domain has 1-5 mismatch nucleotide base pairs. For example, a hairpin stem domain may be have 1, 2, 3, 4 or 5 mismatch nucleotide base pairs.

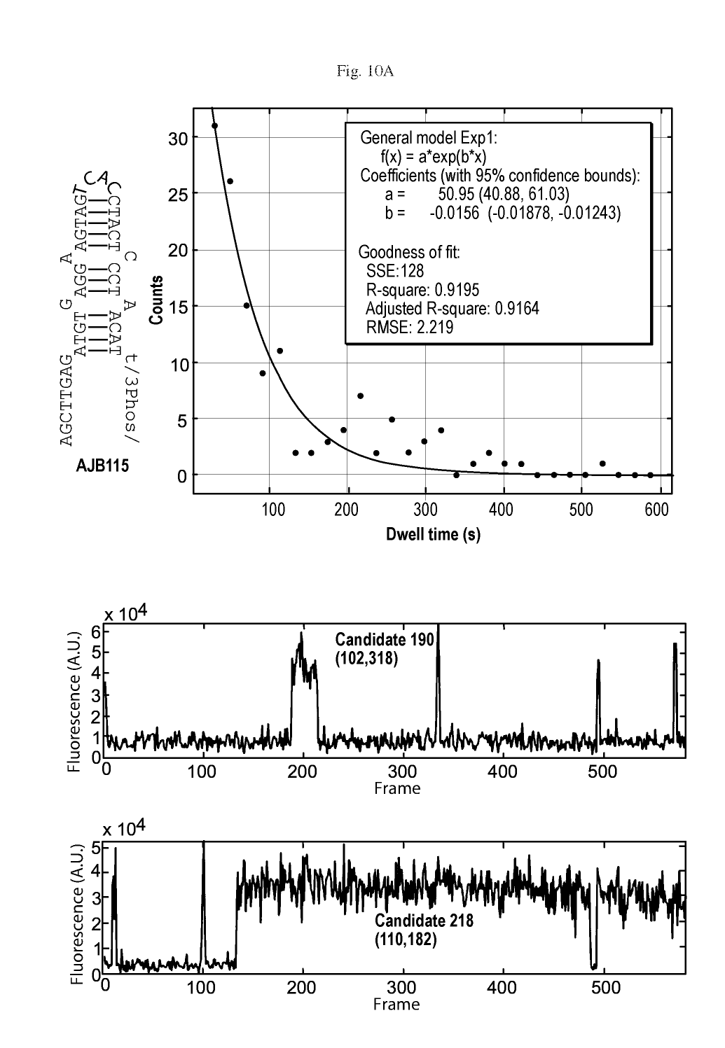

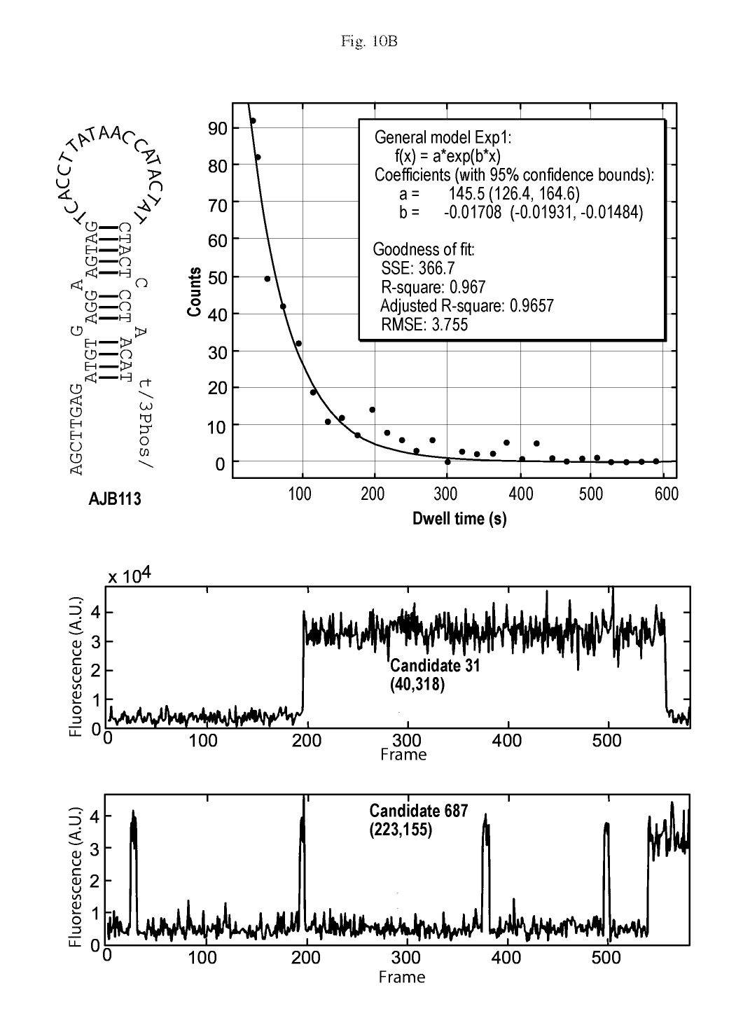

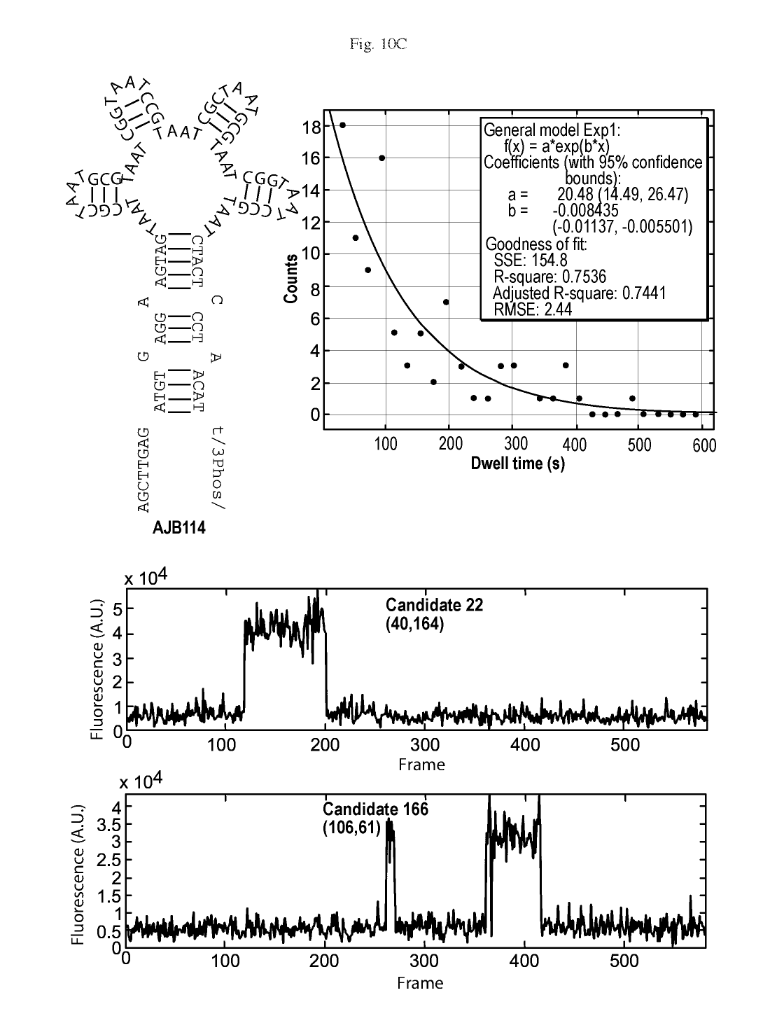

A “hairpin loop domain” refers to a primarily unpaired sequence of nucleotides that form a loop-like structure at the end of the hairpin stem domain. The length of a hairpin loop domain may vary. In some embodiments, an hairpin loop domain has a length 3-200 nucleotides. For example, a hairpin loop domain may have a length of 3-175, 3-150, 3-125, 3-100, 3-75, 3-50, 3-25, 4-175, 4-150, 4-125, 4-100, 4-75, 4-50, 4-25, 5-175, 5-150, 5-125, 5-100, 5-75, 5-50 or 5-25 nucleotides. In some embodiments, a hairpin loop domain has a length of 3-10, 3-15, 32-10, 3-25, 3-30, 3-35, 3-40, 3-35, 3-40, 3-45, 3-50, 4-10, 4-15, 4-10, 4-25, 4-30, 4-35, 4-40, 4-35, 4-40, 4-45 or 4-50 nucleotides. In some embodiments, a hairpin stem domain has a length of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 45, 46, 47, 48, 49 or 50 nucleotides. A hairpin stem domain, in some embodiments, is longer than 300 nucleotides. It should be understood that while hairpin loop in generally described as an unpaired domain, it may have subdomains of intramolecular nucleotide binding. For example, the single-molecule timer depicted in FIGS. 4C, 4D and 10C (left panel) includes four subdomains of intramolecular trinucleotide pairing. These small subdomains with intramolecular base pairing are inserted to prevent long loops of nucleotides from engaging in unintended base pairing with other parts of the template (for instance, the initiator-binding domain or the primer-binding domain). Designing intramolecular base pairing within loops also promotes the formation of a compact, well-defined secondary structure, even for loops with many nucleotides, where conformational entropy or unanticipated base pairing may otherwise make stem formation less favorable. These particular base-pairing patterns shown in the figures were chosen from an iterative series of manual designs with feedback from the freely available NUPACK software package for predicting the secondary structure of nucleic acid sequences.

“Dwell time” refers to the period of time that a primer-template probe complex (e.g., formed by binding of the fluorescently-labeled primer to the template probe) remains bound to the initiator. Binding of a fluorescent-primer-template probe complex to initiator, for example, results in emission (a “pulse”) of a fluorescent signal. The duration of fluorescent signal corresponds with, or is indicative of, dwell time. In some embodiments, dwell time is controlled (varied) by changing the length of the hairpin loop region of the template, as the “clock” graphic indicates in FIG. 1, for example. Thus, dwell time can be increased by lengthening the hairpin loop domain and can be decreased by shortening the hairpin loop domain. In some embodiments, a fluorescent primer-template probe complex binds to an initiator for 5-60 seconds, depending, in part, on the length of the probe (e.g., the length of the hairpin loop domain) and the reaction conditions (e.g., buffer, temperature, dNTP concentration). For example, a fluorescent primer-template probe complex may bind to an initiator for 5-10, 5-10, 5-20, 5-25, 5-30, 5-35, 5-40, 5-45, 5-50 or 5-55 seconds. In some embodiments, a fluorescent primer-template probe complex binds to an initiator for 5, 10, 15, 20, 25, 30, 35, 40, 45, 50, 55 or 60 seconds. In some embodiments, a fluorescent primer-template probe complex binds to an initiator for 5, 6, 7, 8, 9, 10, 11, 12, 14, 15, 16, 17, 18, 19, 20, 21, 22, 23, 24, 25, 26, 27, 28, 29 or 30 seconds.

Dwell time may also be controlled by varying buffer conditions, temperature, and deoxynucleotides (dNTPs) concentrations in a kinetically encoded imaging reaction, as DNA polymerases, like most enzymes, are sensitive to many buffer conditions, including ionic strength, pH and types of metal ions present (e.g., sodium ions vs. magnesium ions). For example, increasing the temperature of a reaction using phi29 polymerase from 15° C. to 30° C. decreases dwell time Likewise, increasing the temperature of a reaction using Bst DNA polymerase, large fragment from room temperature (˜25° C.) to 65° C. (the optimal temperature for polymerization by this enzyme) decreases dwell time. Thus, the temperature at which a kinetically encoded imaging reaction is performed may vary from, for example, 4° C. to 65° C. (e.g., 4° C., 25° C., 37° C., 42° C. or 65° C.). In some embodiments, a kinetically encoded imaging reaction is performed at room temperature, while in other embodiments, a kinetically encoded imaging reaction is performed at 37° C. As another example, increasing salt concentration (e.g., increasing [NaCl] from 40 mM to 200 mM) results in slower DNA polymerization and longer dwell times (e.g., for Bst DNA polymerase, large fragment).

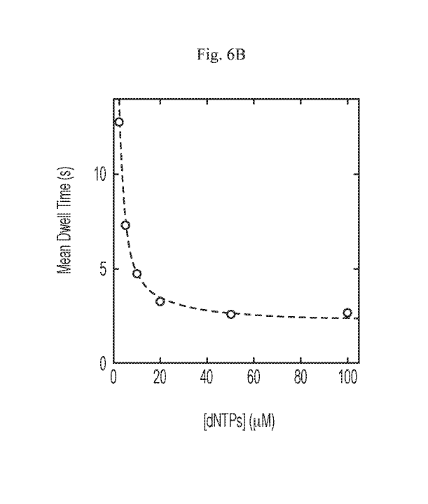

As shown in Example 3 (FIGS. 6A-6B), using a single template design (e.g., 41 nucleotide in length, FIG. 4A (left panel)), it is possible to vary the dwell time of single-molecule timers by varying the concentration of free dNTPs. FIG. 6A presents representative fluorescence time traces of timers operating with the 41-nucleotide template in the presence of 2.5 μM, 5 μM, 10 μM, or 100 μM dNTPs. In the 10 μM dNTP condition, the final 15% of the observation time shows an example of a longer dwell time. This data demonstrates that it is primarily the rate of DNA polymerization that controls the duration of timer binding events. Thus, in some embodiments, the concentration of dNTPs in a kinetically encoded imaging reaction is 100 nM-100 μM. For example, the concentration of dNTPs in a kinetically encoded imaging reaction may be 100, 200, 300, 400, 500, 600, 700, 800, 900 or 1000 nM (1 μM). In some embodiments, the concentration of dNTPs in a kinetically encoded imaging reaction is 2.5 μM-100 μM. For example, the concentration of dNTPs in a kinetically encoded imaging reaction may be 2.5-75, 2.5-50, 2.5-25, 2.5-20, 2.5-5, 5-100, 5-75, 5-50, 5-25, 5-20, 10-100, 10-75, 10-50, 105-25, 10-20, 25-100, 25-75, 25-50, 50-100, 50-75 or 75-100 μM. In some embodiments, the concentration of dNTPs in a kinetically encoded imaging reaction is 0.1, 0.5, 1, 1.5, 2, 2.5, 5, 10, 15, 20, 25, 30, 35, 40, 45, 50, 55, 60, 65, 70, 75, 80, 85, 90, 90 or 100 μM. In some embodiments, the concentration of dNTPs in a kinetically encoded imaging reaction is 2.5 μM to 1 mM, or 2.5 μM to 10 mM. For example, the concentration of dNTPs in a kinetically encoded imaging reaction may be 0.5, 1 mM, 5 mM or 10 mM.

A “primer (P)” refers to an unpaired (single-stranded) nucleic acid that binds to the 3′ subdomain of the hairpin stem domain of a template probe (following binding of the probe to the initiator and dissociation of the stem subdomains). Thus, the length of a primer depends, in part, on the length of the hairpin stem domain. In some embodiments, a primer has a length of 5-40 nucleotides. For example, a primer may have a length of 5-35, 5-30, 5-25, 5-20, 5-15, 5-10, 10-40, 10-35, 10-30, 10-25, 10-20, 10-15, 15-40, 15-35, 15-30, 15-25, 15-20, 20-40, 20-35, 20-30, 20-25, 25-40, 25-35, 25-30, 30-40, 30-35 or 35-40 nucleotides. In some embodiments, a primer has a length of 5, 10, 15, 20, 25, 30, 35 or 40 nucleotides. In some embodiments, a primer has a length of 10, 11, 12, 13, 14, 15, 16, 17, 18, 19, 20, 21, 22, 23, 24 or 25 nucleotides. A primer, in some embodiments, is longer than 40 nucleotides, or shorter than 5 nucleotides.

A primer, as provided herein, may be linked to (labeled with) a detectable molecule (e.g., a molecule that emits a detectable signal, such as a fluorescent or chemiluminescent signal). In some embodiments, the label is a fluorophore. A primer linked to a fluorophore or other fluorescent/chemiluminescent molecule is referred to simply as a “fluorescent primer.” Examples of fluorophores that may be used herein include, without limitation, Hydroxycoumarin, methoxycoumarin, Alexa fluor, aminocoumarin, Cy2, FAM, Alexa fluor 405, Alexa fluor 488, Fluorescein FITC, Alexa fluor 430, Alexa fluor 532, HEX, Cy3, TRITC, Alexa fluor 546, Alexa fluor 555, R-phycoerythrin (PE), Rhodamine Red-X, Tamara, Cy3.5 581, Rox, Alexa fluor 568, Red 613, Texas Red, Alexa fluor 594, Alexa fluor 633, Allophycocyanin, Alexa fluor 647, Cy5, Alexa fluor 660, Cy5.5, TruRed, Alexa fluor 680, Cy7 and Cy7.5. Other fluorophores and molecules that emit a detectable signal are encompassed by the present disclosure.

In some embodiments, a detectable molecule is linked to the template probe rather than the primer. In such embodiments, the primer concentration in a kinetically encoded imaging reaction is high enough that the primer binds immediately after the template probe binds to the initiator nucleic acid. In some embodiments, the concentration of the primer is 100 nM-10 μM. For example, the concentration of the primer may be 100-500 nM, 100-1000 nM, or 100-1500 nM. In some embodiments, the concentration of the primer is 1 μM or at least 1 μM. In some embodiments, the concentration of the primer is 1 μM to 5 μM, or 1 μM to 10 μM.

In some embodiments, a kinetically encoded imaging reaction comprises a fluorescent primer associated with a nucleic acid quencher strand by base pairing (when the fluorescent primer is not bound to the template probe). The proximity of the quencher and the fluorophore in this primer-quencher complex results in reduced fluorescence before the primer binds to the template. This association reduces background fluorescence at a given concentration of primer, which makes it practical to use higher concentrations of the primer, resulting in faster binding kinetics of the primer to the template. To facilitate binding of the primer to the template even when a quencher strand is present, the design includes at least one overhang (or toehold) of 1, 2, 3, 4, 5, 6, 7, 8, 9, 10 or more nucleotides within the primer that do not form base pairs with the quencher strand but which do form base pairs with the template. A “waste complex (W)” refers to the primer-probe complex that results following polymerization (elongation) and which dissociates from the initiator during the final phase of a polymerization reaction. An example of a waste complex is depicted in FIG. 3A.

Kinetically encoded imaging reactions that use polymerase-based single-molecule timers require the use of a polymerase. In some embodiments, the polymerase is a DNA polymerase (DNAP), such as a DNA polymerase having DNA strand displacement activity. “Strand displacement” describes the ability to displace downstream DNA encountered during synthesis. Examples of polymerases having DNA strand displacement activity that may be used as provided herein include, without limitation, phi29 (e.g., NEB #M0269) and Bst DNA polymerase (e.g., NEB #M0275). Phi29 polymerase is most active at moderate temperatures (e.g., 20-37° C.), while Bst polymerase is most active at elevated temperatures (e.g., 65° C.). In some embodiments, the polymerase is an RNA polymerase, reverse transcriptase or a polymerase engineered to incorporate non-natural nucleotides.

FIG. 3A shows a schematic of an example implementation of single-molecule timers using a DNA polymerase (DNAP) having strand displacement activity. A hairpin-loop template (7) binds to an initiator (I) via toehold-mediated strand displacement, revealing the primer-binding domain c. Binding of the fluorescent primer (P) yields a localized increase in fluorescence, and is rapidly followed by binding of DNAP. Elongation of the primer by DNAP serves as the rate-limiting process for dissociation, and results in displacement of I from the fluorescent waste complex (W), which diffuses away and results in a loss of fluorescence. As long as elongation is slow relative to DNAP binding, the duration of the localized spike in fluorescence is controlled (clocked) by the stepwise addition of deoxyribonucleoside triphosphates (dNTPs) to the growing DNA strand. The duplex stem of T, in this example, contains two mismatched base pairs that are introduced to promote orthogonal binding between sequences b/b* and c/c* (to prevent binding of P directly to I).

### Exonuclease-Based Single-Molecule Timers

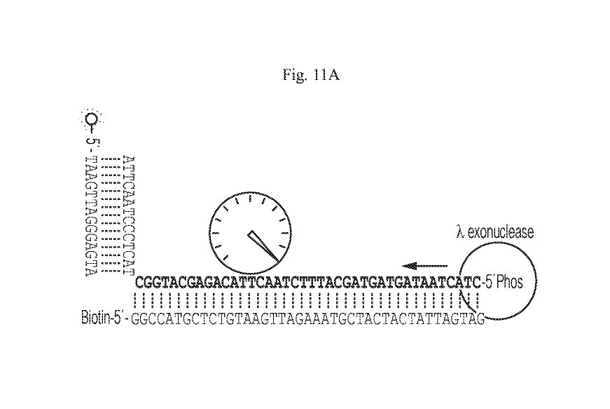

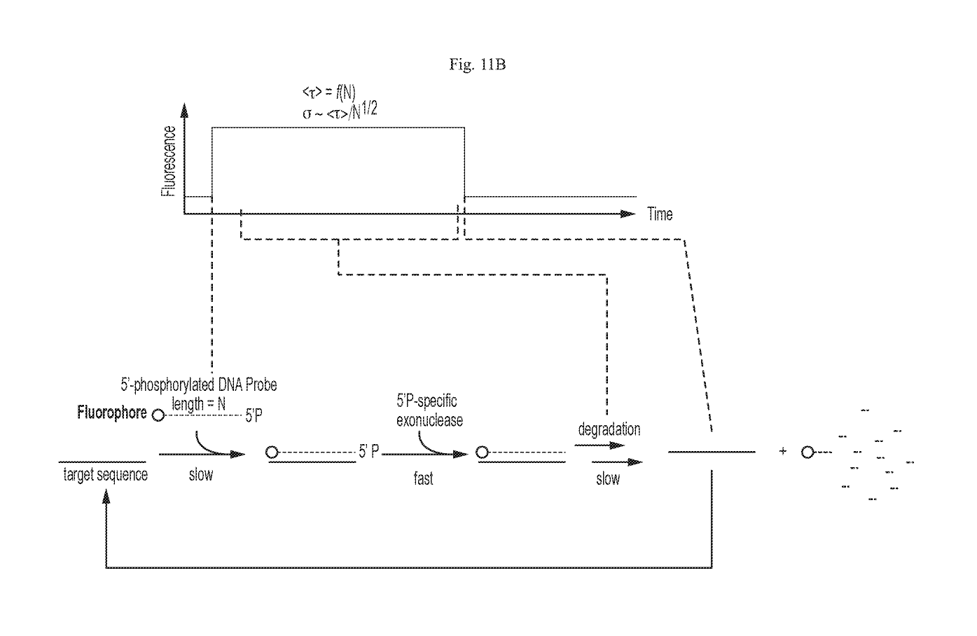

With exonuclease-based timers, an exonuclease that selectively degrades one strand of a nucleic acid duplex is used to generate timer behavior (see, e.g., FIGS. 11A-11B). For example, lambda exonuclease, which selectively degrades the 5′-phosphorylated DNA strand of a DNA duplex, may be used. A fluorescently-labeled DNA probe comprising a 5′-phosphate binds to a target DNA sequence, producing a localized increase in fluorescence. The exonuclease then binds and begins degrading the probe in the 5′-to-3′ direction. Once the exonuclease has degraded enough of the probe to destabilize its interaction with the target, the probe dissociates, resulting in a loss of fluorescence. The duration of the fluorescent signal is thus controlled by the rate of degradation and the length of the probe. Examples of exonucleases that may be used, as provided herein, include, without limitation, T7 exonuclease, E. coli Exonuclease III, RecJf, E. coli Exonuclease I, and Exonuclease T. For example, in some embodiments, the exonuclease is T7 exonuclease. In such embodiments, the initiator nucleic acid may be modified at its 5′-end with a chemical moiety (e.g., a phosphorothioate modification) that renders the initiator non-hydrolyzable.

FIGS. 11A-11B depict examples of exonuclease-based timers that use degradative enzymes rather than polymerases. In FIG. 11A, fluorescently labeled DNA strand binds to the target sequence (initiator). Upon formation of the DNA duplex, the enzyme lambda exonuclease recognizes one of the strands (bearing a 5′-phosphate modification) and begins degrading it from the 5′ end. Lambda Exonuclease does not efficiently recognize single-stranded DNA or non-phosphate-modified DNA, so degradation will primarily happen once the fluorescent strand binds to the target. Once the strand is degraded (after a specified delay), the fluorescent probe will again dissociate, resulting in a loss of localized fluorescence. If degradation kinetics are tuned (e.g., by modifying exonuclease concentration and buffer conditions) such that they are slower than enzyme binding, the rate of disappearance of the fluorescent signal will primarily depend on the degradation process; that is, the degradation will be rate-limiting. In such a scheme, duplexes of different lengths are used to tune the dwell times of single-molecule clocks.

Thus, a kinetically encoded imaging system, in some embodiments, comprises (a) a target nucleic acid, (b) a 5′-phosphorylated nucleic acid probe linked to a 3′ detectable molecule, and (c) a 5′-phosphate-specific exonuclease.

A “5′-phosphorylated nucleic acid probe” refers to an unpaired (single-stranded) nucleic acid that is complementary to and binds to a target sequence of interest. The length of a probe may vary and depends, in part, on the length of the target sequence. In some embodiments, a probe has a length of 5-200 nucleotides. For example, a probe may have a length of 5-190, 5-180, 5-170, 5-160, 5-150, 5-140, 5-130, 5-120, 5-110, 5-100, 5-90, 5-80, 5-70, 5-60, 5-50, 5-40, 5-35, 5-30, 5-25, 5-20, 5-15, 5-10, 10-40, 10-35, 10-30, 10-25, 10-20, 10-15, 15-40, 15-35, 15-30, 15-25, 15-20, 20-40, 20-35, 20-30, 20-25, 25-40, 25-35, 25-30, 30-40, 30-35 or 35-40 nucleotides. In some embodiments, a probe has a length of 5, 10, 15, 20, 25, 30, 35, 40, 45, 50, 55, 60, 65, 70, 75, 80, 85, 90, 95 or 100 nucleotides. In some embodiments, a probe has a length of 10, 11, 12, 13, 14, 15, 16, 17, 18, 19, 20, 21, 22, 23, 24 or 25 nucleotides. A probe, in some embodiments, is longer than 200 nucleotides, or shorter than 5 nucleotides.

A 5′-phosphorylated nucleic acid probe, as provided herein, may be linked to (labeled with) a detectable molecule. In some embodiments, the label is a fluorophore. A probe linked to a fluorophore or other fluorescent/chemiluminescent molecule is referred to simply as a “fluorescent probe.” Examples of fluorophores that may be used herein include, without limitation, Hydroxycoumarin, methoxycoumarin, Alexa fluor, aminocoumarin, Cy2, FAM, Alexa fluor 405, Alexa fluor 488, Fluorescein FITC, Alexa fluor 430, Alexa fluor 532, HEX, Cy3, TRITC, Alexa fluor 546, Alexa fluor 555, R-phycoerythrin (PE), Rhodamine Red-X, Tamara, Cy3.5 581, Rox, Alexa fluor 568, Red 613, Texas Red, Alexa fluor 594, Alexa fluor 633, Allophycocyanin, Alexa fluor 647, Cy5, Alexa fluor 660, Cy5.5, TruRed, Alexa fluor 680, Cy7 and Cy7.5. Other fluorophores and molecules that emit a detectable signal are encompassed by the present disclosure.

Exonuclease-based single-molecule timers may be used in a kinetically encoded imaging method. In some embodiments, the method comprises combining in reaction buffer (a) a target nucleic acid, (b) a 5′-phosphorylated nucleic acid probe linked to a 3′ detectable molecule, and (c) a 5′-phosphate-specific exonuclease; and incubating the reaction mixture under conditions that result in exonuclease-mediated degradation of the probe.

### Hybridization Cascade Single-Molecule Timers

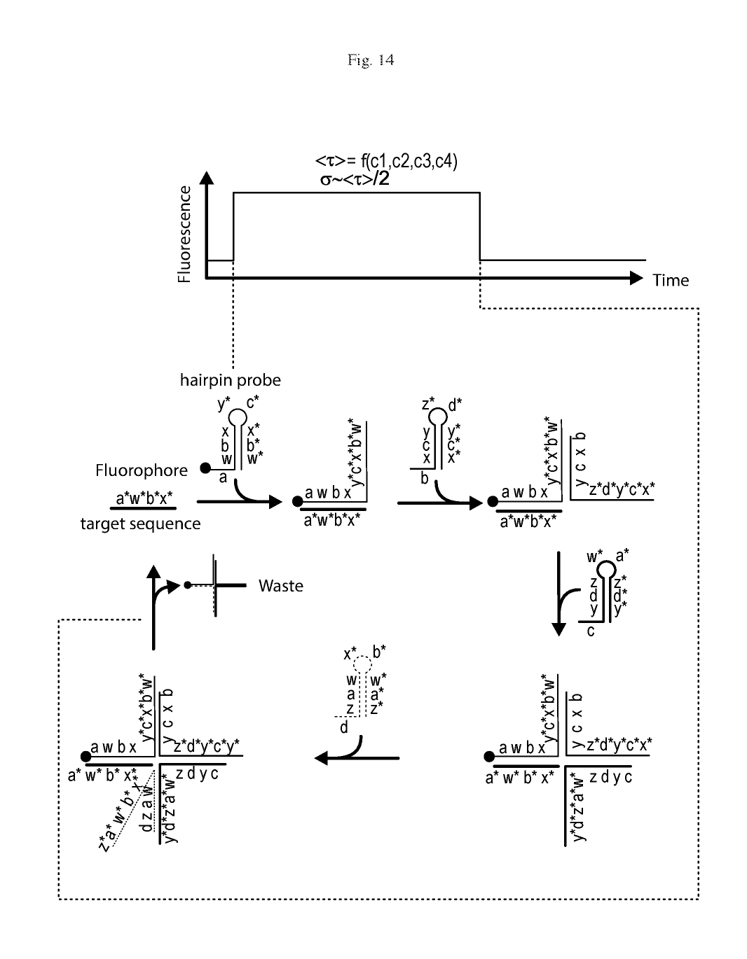

With hybridization cascade single-molecule timers, a series of nucleic acids react with each other in a specific order, resulting in the production of a branched Waste Complex (see, e.g., FIG. 14). As shown in FIG. 14, the toehold domain of hairpin (stem-loop) nucleic acid probe hybridizes with an initiator (target) nucleic acid, resulting in opening of the hairpin probe, exposing nucleotides of the hairpin loop and stem domains. The initial hairpin probe is linked to a fluorescent molecule such that a localized increase in fluorescence is observed upon binding of the probe to the initiator nucleic acid. Next, the toehold domain of a second hairpin probe hybridizes with the newly exposed nucleotides of the hairpin loop and stem domains of the first probe, resulting in opening of the second hairpin probe, exposing nucleotides of the hairpin loop and stem domains of the second hairpin probe. The toehold domain of a third hairpin probe then hybridizes with the newly exposed nucleotides of the hairpin loop and stem domains of the second hairpin probe, resulting in opening of the third hairpin probe, exposing nucleotides of the hairpin loop and stem domains of the third hairpin probe. Finally, the toehold domain of a fourth hairpin probe hybridizes with the newly exposed nucleotides of the hairpin loop and stem domains of the third hairpin probe, resulting in opening of the fourth hairpin probe. The nucleotides of the hairpin loop and stem domains of the fourth hairpin probe then bind to the first hairpin probe, displacing the initiator nucleic acid, resulting in dissociation of the fluorescent 4-arm waste complex and disappearance of the localized fluorescence. The delay between the appearance of fluorescence (with binding of the first template probe to the initiator nucleic acid) and the disappearance of fluorescence (with dissociation of the waste complex) follows a gamma distribution rather than an exponential distribution, similar to the gamma distribution of the polymerase-based single-molecule timers. Advantageously, single-molecule timers constructed from hybridization cascades do not require the use of an enzyme (e.g., polymerase or exonuclease) and, thus, are compatible with a wider variety of conditions (e.g., salt concentrations, temperatures, pH) relative to the enzyme-based timers.

Thus, a kinetically encoded imaging system, in some embodiments, comprises (a) an unpaired initiator nucleic acid, and (b) a first hairpin probe, a second hairpin probe, a third hairpin probe and a fourth hairpin probe, each hairpin probe comprising (i) an unpaired 5′ toehold domain, (ii) a hairpin stem domain formed by intramolecular base pairing between nucleotides located in a 5′ subdomain of the probe and nucleotides located in a 3′ subdomain of the probe, and a hairpin loop domain located between the 5′ subdomain and the 3′ subdomain, wherein the first hairpin probe is linked to a detectable molecule.

In some embodiments, (i) the toehold domain and the 5′ subdomain of the first probe are complementary to and bind to the initiator nucleic acid, (ii) the toehold domain and the 5′ subdomain of the second probe are complementary to and bind to the hairpin stem domain and the 3′ subdomain of the first probe bound to the initiator sequence, (iii) the toehold domain and the 5′ subdomain of the third probe are complementary to and bind to the hairpin stem domain and the 3′ subdomain of the second probe bound to the first probe, and (iv) the toehold domain and the 5′ subdomain of the fourth probe are complementary to and bind to the hairpin stem domain and the 3′ subdomain of the third probe bound to the second probe, and wherein the hairpin loop and 3′ subdomain of the fourth probe are complementary to and bind to the toehold domain and the 5′ subdomain of the first probe.

Hybridization cascade single-molecule timers may be used in a kinetically encoded imaging method. In some embodiments, the method comprises combining in reaction buffer (a) an unpaired initiator nucleic acid, and (b) a first hairpin probe, a second hairpin probe, a third hairpin probe and a fourth hairpin probe, each hairpin probe comprising (i) an unpaired 5′ toehold domain, (ii) a hairpin stem domain formed by intramolecular base pairing between nucleotides located in a 5′ subdomain of the probe and nucleotides located in a 3′ subdomain of the probe, and a hairpin loop domain located between the 5′ subdomain and the 3′ subdomain, wherein the first hairpin probe is linked to a detectable molecule, and incubating the reaction mixture under conditions that result in DNA hybridization.

### Nucleic Acids

It should be understood that the nucleic acids of the present disclosure do not occur in nature. Thus, the nucleic acids may be referred to as “engineered nucleic acids.” An “engineered nucleic acid” is a nucleic acid (e.g., at least two nucleotides covalently linked together, and in some instances, containing phosphodiester bonds, referred to as a phosphodiester “backbone”) that does not occur in nature. Engineered nucleic acids include recombinant nucleic acids and synthetic nucleic acids. A “recombinant nucleic acid” is a molecule that is constructed by joining nucleic acids (e.g., isolated nucleic acids, synthetic nucleic acids or a combination thereof) and, in some embodiments, can replicate in a living cell. A “synthetic nucleic acid” is a molecule that is amplified or chemically, or by other means, synthesized. A synthetic nucleic acid includes those that are chemically modified, or otherwise modified, but can base pair with (also referred to as “binding to,” e.g., transiently or stably) naturally-occurring nucleic acid molecules. Recombinant and synthetic nucleic acids also include those molecules that result from the replication of either of the foregoing.

While an engineered nucleic acid, as a whole, is not naturally-occurring, it may include wild-type nucleotide sequences. In some embodiments, an engineered nucleic acid comprises nucleotide sequences obtained from different organisms (e.g., obtained from different species). For example, in some embodiments, an engineered nucleic acid includes a murine nucleotide sequence, a bacterial nucleotide sequence, a human nucleotide sequence, a viral nucleotide sequence, or a combination of any two or more of the foregoing sequences.

In some embodiments, an engineered nucleic acid of the present disclosure may comprise a backbone other than a phosphodiester backbone. For example, an engineered nucleic acid, in some embodiments, may comprise phosphoramide, phosphorothioate, phosphorodithioate, O-methylphophoroamidite linkages, peptide nucleic acids or a combination of any two or more of the foregoing linkages. An engineered nucleic acid may be single-stranded (ss) or double-stranded (ds), as specified, or an engineered nucleic acid may contain portions of both single-stranded and double-stranded sequence. In some embodiments, an engineered nucleic acid contains portions of triple-stranded sequence, or other non-Watson-Crick base pairing such as G-quartets, G-quadruplexes, and i-motifs. An engineered nucleic acid may comprise DNA (e.g., genomic DNA, cDNA or a combination of genomic DNA and cDNA), RNA or a hybrid molecule, for example, where the nucleic acid contains any combination of deoxyribonucleotides and ribonucleotides (e.g., artificial or natural), and any combination of two or more bases, including uracil, adenine, thymine, cytosine, guanine, inosine, xanthine, hypoxanthine, isocytosine and isoguanine.

Engineered nucleic acids of the present disclosure may be produced using standard molecular biology methods (see, e.g., Green and Sambrook, Molecular Cloning, A Laboratory Manual, 2012, Cold Spring Harbor Press). In some embodiments, nucleic acids are produced using GIBSON ASSEMBLY® Cloning (see, e.g., Gibson, D.G. et al. Nature Methods, 343-345, 2009; and Gibson, D.G. et al. Nature Methods, 901-903, 2010, each of which is incorporated by reference herein). GIBSON ASSEMBLY® typically uses three enzymatic activities in a single-tube reaction: 5′ exonuclease, the 3′ extension activity of a DNA polymerase and DNA ligase activity. The 5′ exonuclease activity chews back the 5′ end sequences and exposes the complementary sequence for annealing. The polymerase activity then fills in the gaps on the annealed domains. A DNA ligase then seals the nick and covalently links the DNA fragments together. The overlapping sequence of adjoining fragments is much longer than those used in Golden Gate Assembly, and therefore results in a higher percentage of correct assemblies. Other methods of producing engineered nucleic acids are known in the art and may be used in accordance with the present disclosure.

### Kinetic Encoding and Compact Barcoding

An individual single-molecule timer exhibits characteristic changes in fluorescence over time, with intensity spikes (“ON”) that occur at random intervals amid a low-fluorescence background (“OFF”) but have a precisely controlled duration (see, e.g., FIG. 3B). By varying the characteristic ON-state dwell time of a timer system (e.g., by changing the length of the template in a polymerase-based timer), multiple timers that are distinguishable by kinetics alone due to their well-separated dwell time distributions can construct. In a polymerase-based timer system, the mean dwell time is linearly dependent on the length of the template (see, e.g., FIG. 4F), allowing the systematic design of multiple distinguishable timers. For multiplexed detection, several timers with well-separated lifetime distributions can be used in concert to create kinetic barcodes that can be read out in minutes. For example, each timer responds to a target sequence that is either present or absent from a label sequence appended to each target of interest. The presence or absence of each timer constitutes one “bit” of information in the barcode. Thus, a single-color system with six timer lifetimes has the potential to distinguish 2⁶−1=63 unique targets (the number 1 is subtracted because the all-negative barcode is undetectable). Using three colors, a six-lifetime system can distinguish (2³)⁶−1=262,143 targets. This high degree of multiplexing does not require any kind of strand exchange or spatial encoding of information. In the embodiments discussed above, each barcode may be only tens of nanometers in size, making super-resolution imaging within intact cells and tissues a possibility.

### Rapid, One-Pot Multiplexing

Unlike existing approaches to multiplexing in fluorescence microscopy, barcoding with single-molecule timers does not require multiple buffer exchanges, photobleaching steps, or ex situ imaging. Rather, the sample can be imaged directly in the presence of the mixture of timers, because they all can be distinguished by a combination of dwell time and wavelength. This imaging may take place over minutes or tens of minutes, rather than the several hours or days required for competing approaches.

### Multiplexed Super-Resolution Microscopy of Cells and Tissues

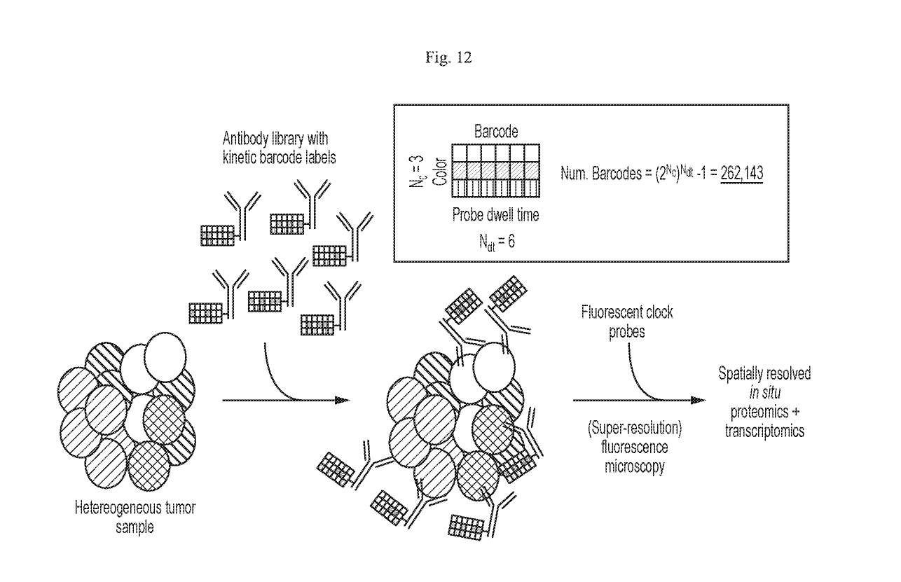

Single-molecule timers may be used, in some embodiments, for multiplexed super-resolution microscopy of cells and tissues (see, e.g., FIG. 12). Kinetic barcodes constructed from single-molecule timers can used to detect and spatially resolve thousands of distinct targets in a single imaging experiment lasting only 10-30 minutes. The target biomolecules may be proteins, nucleic acids, or any other biomolecule for which selective affinity reagents (e.g., antibodies or hybridization probes) are available and may be conjugated to appropriate barcode sequences. In one embodiment, a DNA-barcoded antibody library is constructed; that is, each antibody species is associated with a unique DNA barcode containing some combination of initiator sequences. Each barcode, and hence, each antibody, is identifiable based on the combination of timer probes that bind to it. Furthermore, because single binding events are detected and can be localized using various existing fitting algorithms, super-resolution microscopy is possible. This technique can be used, for instance, in the high-content imaging of heterogeneous tissues, such as tumors, where the expression levels and localization of biomarkers within the tissue may help to characterize and respond to drug resistance.

### Single-Molecule Clocks

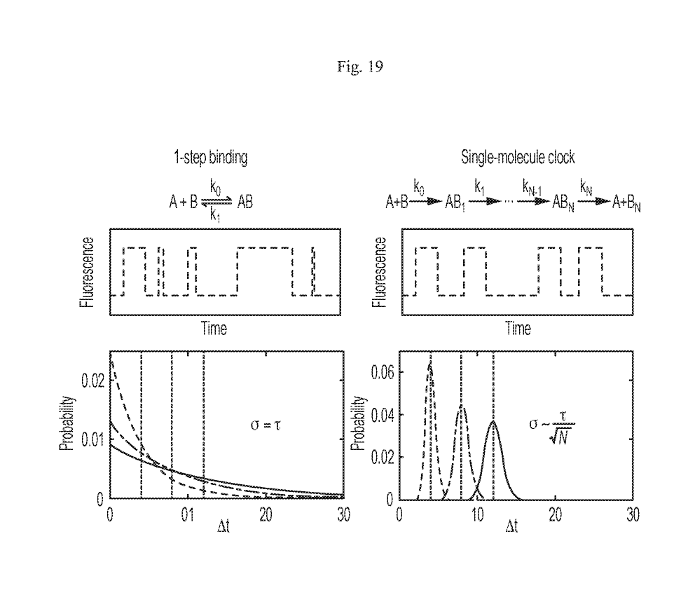

Single-molecule clocks, as provided herein, in some aspects, utilize a cascade of several irreversible reactions to establish a well-defined time delay between repeat binding, dissociation, or any combination thereof, of a fluorescent imager strand or strands to a nucleic acid generated or displaced from a complementary template; the summation of several exponentially distributed variables yields a sharp gamma distribution of dwell times (see, e.g., FIG. 19 (right panel)). That is, each binding event has a precisely determined duration, with a degree of randomness that decreases in proportion to

$$\frac{1}{\sqrt{N}} ,$$

where N is the number of reversible reactions between binding and dissociation. This quasi-deterministic behavior permits the unambiguous identification of a target by observing the duration of a single binding event.

### Principles of Single-Molecule Clocks

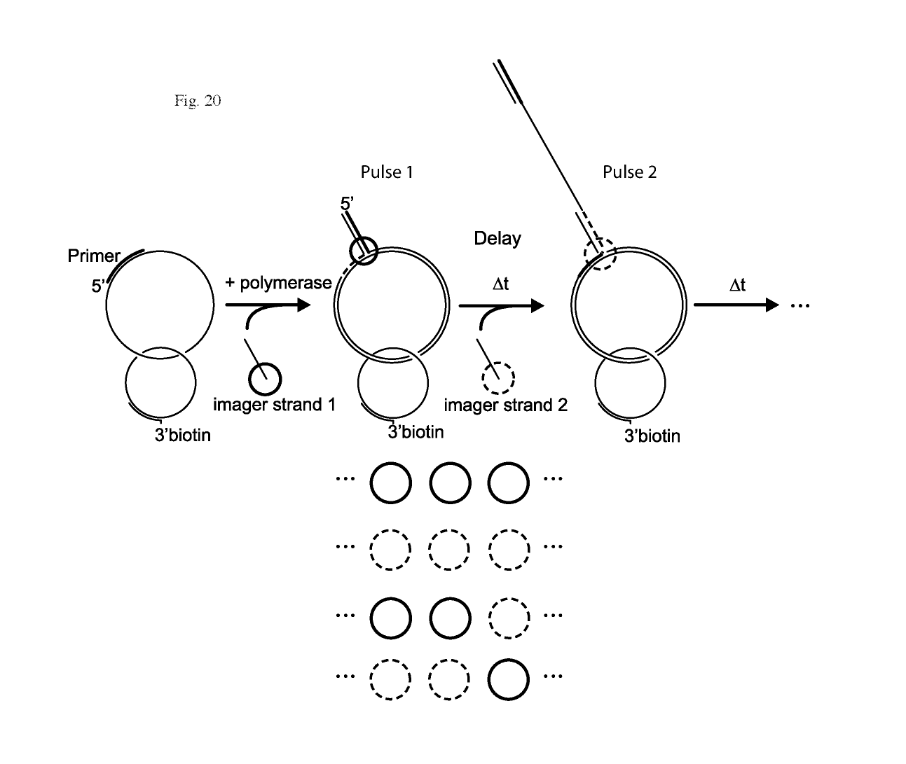

The binding equilibrium of a bimolecular complex can usually be approximated as a 1-step binding system (or a two-state system) characterized by a bimolecular association rate constant kₒ and a unimolecular dissociation rate constant k₁ (FIG. 19, left panel). Thus, when the reversible binding of a fluorescent probe to an immobilized target is monitored at the single-molecule level, the dwell times in the high-fluorescence (bound) state are exponentially distributed, with a standard deviation equal to the mean dwell time μ (FIG. 1, left panel). Effectively, the complex has no memory of how long it has existed, and its probability of dissociating within an interval of time Δt is independent of its history, resulting in a broad range of complex lifetimes. In contrast, single-molecule clocks pass through a series of intermediates via reversible reactions characterized by rate constants k₁, k₂, . . . k_{N} before a subsequent binding or dissociation event can occur (FIG. 20, right panel). While the lifetime of each intermediate is exponentially distributed, the sum

$${τ}_{N} = {\sum }_{i = 1}^{N} \frac{1}{{k}_{i}} \qquad ( 1 )$$

obeys a gamma distribution, with standard deviation proportional to 1/\sqrt{N} (FIG. 19, right panel). That is, as the number of rate-limiting reversible steps increases, the lifetime of the complex becomes more narrowly distributed about its expectation value, or more deterministic.

Single-molecule clocks enable the generation of periodic fluorescent signals. A DNA polymerase is used in combination with a circular DNA template to generate delays between signaling events. As shown in FIG. 20, the circular template (larger black circle, top) is mechanically interlocked with another circular DNA strand, referred to as a ‘leash’ (smaller black circle, bottom). The leash allows the template to be physically linked to an affinity tag (e.g., biotin, an antibody or a hybridization probe) while not interfering with access of the polymerase to the template. To generate periodic signals, a primer (short light gray line) is hybridized to the template, and then a DNA polymerase (e.g., phi29 polymerase) is added to the reaction. The polymerase begins extending the primer via rolling circle amplification (RCA), a mechanism of synthesizing long (>10,000 nucleotides) DNA products from a circular template. At defined times, in a manner specified by the DNA sequence of the product, fluorescent pulses are generated. The delay time between pulses is determined by the number of nucleotides synthesized in between pulses as well as the rate of polymerization, which depends, for example, on the polymerase properties, buffer conditions, nucleotide concentration and temperature of the reaction. This method of generating periodic fluorescent signals from a cyclic reaction cascade is referred to herein as a periodic single-molecule clock. Within each cycle, there may be a single fluorescent pulse or multiple pulses.

Signal is generated by the binding of a short fluorescent DNA imager strand (e.g., ˜20 nucleotides) to the product DNA sequence as it is liberated by the strand displacement activity of the polymerase. The imager strand, in some embodiments, is present at high concentration (e.g., ˜5 micromolar) so that imager strand binding is fast compared to the lifetime of a period (the time it takes for the polymerase to synthesize one copy from the template sequence). In some embodiments, other signal-generating mechanisms may be used, such as the addition of fluorescent or fluorogenic nucleotides.

The length of delays between these pulses can be used to encode the identity of a molecular target that is bound by the leash. This type of encoding scheme may be useful for multiplexed fluorescence microscopy, for example.

In addition to time delays between fluorescent pulses, target identity can also be encoded in the order of multicolor probe binding events. For instance, by embedding two different probe sequences in the template, each specifying a different color (e.g., red and blue), a variety of permutations of color and order should be possible (see, e.g., FIG. 20). A still greater number of permutations using 3, 4 or more colors is encompassed herein. If a long intervening sequence is inserted to indicate the start of each period, circular permutations of colors become distinguishable, and a larger number of distinct pulse sequences are possible.

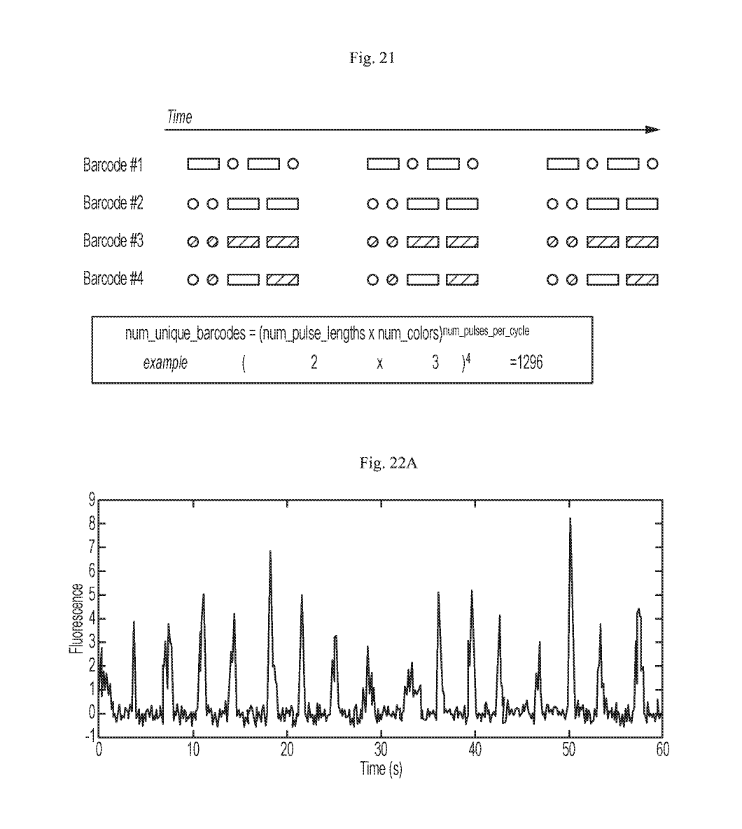

Further, by combining both time delays and multiple probe colors, an encoding scheme resembling a “multicolor Morse code” may be used (see, e.g., FIG. 21). For instance, with two distinct pulse delays and three probe colors, 1296 distinct pulse sequences are possible, allowing the simultaneous imaging of 1296 molecular targets.

Morse Probes. In some embodiments, a “Morse probe” labeling system with a catenated DNA structure may be used. Morse probe systems provide a low signal-to-noise ratio due to at least two features. First, only a single imager strand should bind to a template at any given time. Therefore, only a single fluorophore emits a fluorescent signal (for a given probe) at any given time, which greatly limits sources of noise, such as double (imager) binding events. Second, in order for the polymerase to move forward, it displaces the imager strand, which enables a dark state before the arrival of the next imager strand and obviates a need to photobleach the fluorophore, or to wait for quencher strand binding to achieve the dark state.

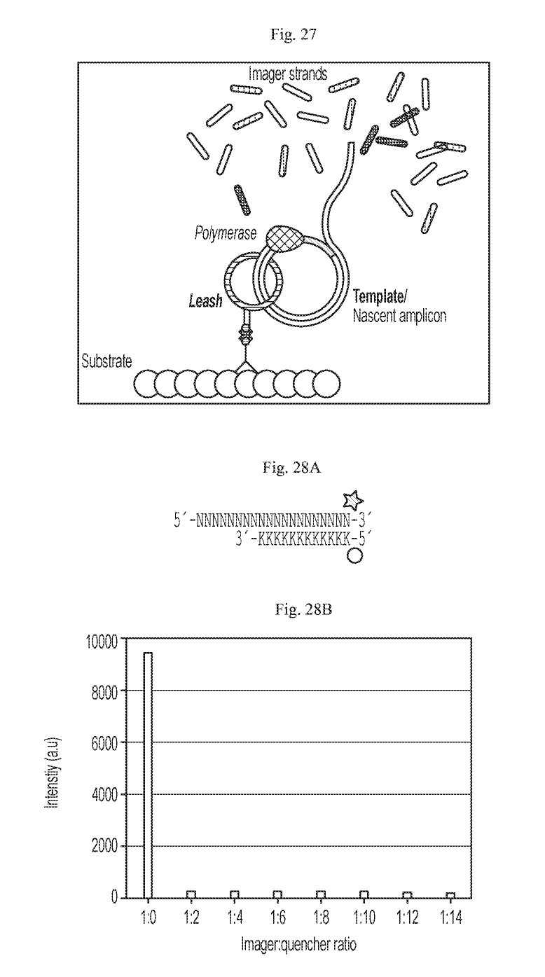

A schematic of an example of a Morse probe labeling system is shown in FIG. 27. In this example, a leash is linked to a target of interest (substrate; or example, protein or RNA) using biotin (small gray circles) and streptavidin (dark gray cross). Other means of linking a leash to a substrate may be used. Primer sequences and dNTPs are included in the reaction solution; binding of the primer to the template provides the site of replication initiation. Strand displacement DNA polymerase replicates a circular template (inner dark gray circle) DNA strand using the dNTPs from the solution to produce the nascent amplicon (product; shown in black). The strand displacing activity of the polymerase and circular nature of the template enable continuous replication. A single-stranded DNA site having a length of 3-5 nucleotides (toehold, which may be longer than 5 nucleotides), for example, opens in front of the polymerase (Morin et al., Proceedings of the National Academy of Sciences, 2012, 109: 8115-20), providing a binding site for a fluorescently labeled imager strand to attach to the template through strand displacement.

Imager strands may be present in high concentration in solution, in some embodiments, in order to facilitate favorable kinetics (Zhang et al., J Am Chem Soc., 2009, 131: 17303-14). To minimize background fluorescence resulting from the presence of imager strands at high concentrations, the imager strands may be hybridized with a complementary strand containing a quencher, in a molecular beacon type configuration. Once the imager strand attaches to the template, the quencher is displaced, resulting in emission of a fluorescent signal (fluorescent pulse). A short time thereafter, the polymerase reaches the site of attachment and displaces the imager strand, bringing the system back to its “dark” state.

The imager strand binds in front of the polymerase and, therefore, the directionality of the polymerase prescribes which end (3′ end or 5′ end) of the probe comprises the label (e.g., fluorophore). In some embodiments, a fluorescent label is located at the 3′ end of the imager strand, while the quencher molecule is located at the 5′ end of a corresponding (complementary) quencher strand. In some embodiments, the imager strand is 5-20 nucleotides longer than its corresponding quencher strand. In such embodiments, this length limits the fluorophore of the imager strand to one end (e.g., the 3′ end) and the fluorophore of the quencher strand to the other end (e.g., 5′ end).

Examples of quencher molecules include, but are not limited to, the following: BHQ 0, BHQ 1, BHQ 2, BHQ 3, Iowa Black FQ, and Iowa Black RQ. Other quencher molecules may be used. Each molecule quenches a fluorescent signal within a characteristic range; however, the system is agnostic to the choice of fluorophore-quencher pair and therefore, as more quenchers become available, these other quenchers may be used. The ratio of imager strand:quencher strand should be sufficient so that there is minimal background fluorescence during TIRF imaging. The imager:quencher ratio can be, for example, 1:2, 1:4, 1:6, 1:8, 1:10, 1:12, or 1:14. In some embodiments, the imager:quencher ratio is 1:4. The imager strand:quencher strand ratio may vary depending on the particular reaction conditions used.

In some embodiments, the leash is ssDNA or dsDNA. The outcome is independent of the length of the leash, but the leash is typically long enough such that the lumen of the circular leash allows the passage of polymerase without restriction.

In some embodiments, the template (template strand) is ssDNA. The length of the template in part determines the number of binding sites, which provides the encoding capacity of the Morse probe library. The GC content of the template, salt concentration in buffers, imaging temperature, and concentration of dNTPs in solution are some of the parameters that contribute to the speed of polymerase activity. Thus, these parameters also contribute toward the speed of attachment and detachment of the imager strand and the distribution of wait times.

In some embodiments, an endonuclease may be introduced to cleave the product strand, so that the imager strand has unfettered access to bind to the template. In time-lapse imaging, the large product strand may clump to the point where it limits the imager strand's access to the template strand, which may limit the time a system can be studied. With the inclusion of an endonuclease, little to no binding will occur on the product strand and imaging should not be adversely affected.

In some embodiments, the binding sites for imager strands may partially overlap on the template. This works because the number of nucleotides “ahead” of the polymerase is small enough (3-5 nucleotides) to ensure binding of only one strand at a time. Once one strand is displaced by the polymerase, a separate, partially overlapping binding site can become available. Therefore, the coding capacity for a template of a given length is increased.

### Single-Molecule Clock Components

Circular Nucleic Acid Template

Single-molecule clocks of the present disclosure are architecturally similar to a catenane: a mechanically-interlocked molecular structure that includes at least two interlocked macrocycles. A single-molecule clock (“clock”) comprises a circular nucleic acid “template strand” (or “template”) interlocked with a circular nucleic acid “leash strand.” A circular nucleic acid may be a single-stranded or double-stranded nucleic acid joined at each end (e.g., the 5′ and the 3′ end of the strand joined to each other via ligation) to form a circular structure. As example of a single-molecule clock catenane is shown in FIG. 20, where the large circle represents template and the small circle represents the leash.

A template typically includes a primer binding sequence. A primer binding sequence is a sequence of nucleotides (e.g., comprising A, T, C and G) to which a nucleic acid primer can bind. Thus, a nucleic acid primer comprises a nucleotide sequence complementary to a primer binding sequence. The length of a primer binding sequence (and thus the complementary primer) may vary. In some embodiments, a primer binding sequence has a length of 5-50 nucleotides. For example, a primer binding sequence may have a length of 5-45, 5-40, 5-35, 5-30, 5-25, 5-20, 5-15, 5-10, 10, 40, 10-45, 10-40, 10-35, 10-30, 10-25, 10-20, 10-15, 15-50, 15-45, 15-40, 15-35, 15-30, 15-25, 15-20, 20-50, 20-45, 20-40, 20-35, 20-30, 20-25, 25-40, 25-35, 25-30, 30-50, 30-45, 30-40, 30-35 or 35-40 nucleotides. In some embodiments, a primer binding sequence has a length of 5, 10, 15, 20, 25, 30, 35, 40, 45 or 50 nucleotides. In some embodiments, a primer binding sequence has a length of 10, 11, 12, 13, 14, 15, 16, 17, 18, 19, 20, 21, 22, 23, 24 or 25 nucleotides. A primer binding sequence, in some embodiments, is longer than 50 nucleotides.

A circular nucleic acid template, in some embodiments, comprises deoxyribonucleic acid (DNA), ribonucleic acid (RNA), locked nucleic acid (LNA), peptide nucleic acid (PNA), or a combination thereof. In some embodiments, a template comprises DNA.

The length of a circular template may vary. It should be understood that “length” in the context of a circular structure refers to the number of contiguous nucleotides in the structures (the number of nucleotides that form the circular structure). In some embodiments, the length of a template is 50-10000 nucleotides. For example, a template may have a length of 50-5000, 50-1000, 50-500, 50-200, 50-100, 100-10000, 100-5000, 100-1000, 100-500, 100-200, 200-10000, 200-5000, 200-1000, 200-500, 500-10000, 500-5000, or 500-1000 nucleotides. In some embodiments, a template has a length of 50, 100, 200, 300, 400, 500, 600, 700, 800, 900, 1000, 2000, 3000, 4000, 5000, 6000, 7000, 8000, 9000 or 10000 nucleotides.

Product Strand

A template may also include a nucleotide sequence that “specifies” or “encodes” an imager strand binding sequence. It should be understood that in the context of the present disclosure, a nucleotide sequence of a template is considered to “specify” or “encode” its complementary strand, e.g., produced via a rolling circle replication reaction. This complementary strand is referred to herein as the “product strand.” Thus, as shown, for example, in FIG. 20, a primer binds to a template and (in the presence of polymerase and dNTPs under conditions suitable for nucleic acid polymerization) the product strand (a strand complementary to the template) is synthesized. This newly synthesized product strand may contain an imager strand binding sequence (a sequence to which an imager strand binds), which was specified by the template.

In some embodiments, a primer binding sequence “specifies” or “encodes” an imager strand binding sequence. That is, an imager strand, in some embodiments, may bind to a sequence on a product strand that is specified by the primer binding sequence (is complementary to the primer binding sequence).

The length of a product strand may also vary. In some embodiments, the length of a product strand is 50-10000 nucleotides. For example, a product strand may have a length of 50-5000, 50-1000, 50-500, 50-200, 50-100, 100-10000, 100-5000, 100-1000, 100-500, 100-200, 200-10000, 200-5000, 200-1000, 200-500, 500-10000, 500-5000, or 500-1000 nucleotides. In some embodiments, a product strand has a length of 50, 100, 200, 300, 400, 500, 600, 700, 800, 900, 1000, 2000, 3000, 4000, 5000, 6000, 7000, 8000, 9000 or 10000 nucleotides. In some embodiments, a product strand is longer than 10000 nucleotides.

Circular Nucleic Acid Leash

A circular nucleic acid leash (“leash”) enables the template to be physically linked to an affinity tag while not interfering with access of a polymerase to the template. Thus, in some embodiments, a circular nucleic acid leash is linked to (bound to) a binding partner molecule, such as biotin, a ligand, a receptor, an antibody or a nucleic acid (e.g., hybridization probe). Other binding partner molecules (molecules that specifically bind to other molecules) are encompassed herein.

The length of a circular nucleic acid leash may also vary. In some embodiments, the length of a leash is 50-10000 nucleotides. For example, a leash may have a length of 50-5000, 50-1000, 50-500, 50-200, 50-100, 100-10000, 100-5000, 100-1000, 100-500, 100-200, 200-10000, 200-5000, 200-1000, 200-500, 500-10000, 500-5000, or 500-1000 nucleotides. In some embodiments, a leash has a length of 50, 100, 200, 300, 400, 500, 600, 700, 800, 900, 1000, 2000, 3000, 4000, 5000, 6000, 7000, 8000, 9000 or 10000 nucleotides. In some embodiments, a leash is shorter than a template. In some embodiments, a leash has a length of less than 50 nucleotides. For example, a leash may have a length of 10, 15, 20, 25, 30, 35, 40 or 45 nucleotides. In some embodiments, a leash has a length of 5-100 nucleotides, 10-100 nucleotides, or 20-100 nucleotides.

A circular nucleic acid leash, in some embodiments, comprises deoxyribonucleic acid (DNA), ribonucleic acid (RNA), locked nucleic acid (LNA), peptide nucleic acid (PNA), or a combination thereof. In some embodiments, a circular nucleic acid leash comprises DNA. In some embodiments, a circular nucleic acid leash comprises RNA.

In some embodiments, a non-nucleic acid leash is used to enable the template to be physically linked to an affinity tag while not interfering with access of a polymerase to the template. For example, a leash may be a polypeptide or other cyclic organic molecule, provided (1) the leash can be mechanically interlocked with the template, (2) the leash is large enough to permit passage of the polymerase (e.g., diameter greater than ˜5 nanometers), and (3) the leash can be chemically linked to an antibody or other affinity reagent.

Primer

Typically, a primer is an unpaired (single-stranded) nucleic acid (e.g., DNA), although, in some instances, a primer may be partially paired (partially double-stranded) (containing a paired domain and an unpaired domain). A primer comprises a nucleotide sequence that is complementary to a primer binding sequence of a template and can bind to a template to initiate polymerization (in the presence of polymerase and dNTPs). In some embodiments, a primer is a single strand of DNA. Thus, the length of a primer depends, in part, on the length of the primer binding sequence on the template. In some embodiments, a primer has a length of 5-50 nucleotides. For example, a primer may have a length of 5-45, 5-40, 5-35, 5-30, 5-25, 5-20, 5-15, 5-10, 10, 40, 10-45, 10-40, 10-35, 10-30, 10-25, 10-20, 10-15, 15-50, 15-45, 15-40, 15-35, 15-30, 15-25, 15-20, 20-50, 20-45, 20-40, 20-35, 20-30, 20-25, 25-40, 25-35, 25-30, 30-50, 30-45, 30-40, 30-35 or 35-40 nucleotides. In some embodiments, a primer has a length of 5, 10, 15, 20, 25, 30, 35, 40, 45 or 50 nucleotides. In some embodiments, a primer has a length of 10, 11, 12, 13, 14, 15, 16, 17, 18, 19, 20, 21, 22, 23, 24 or 25 nucleotides. A primer, in some embodiments, is longer than 50 nucleotides.

Polymerase

Compositions and methods of the present disclosure, in some embodiments, include a polymerase. In some embodiments, the polymerase is a DNA polymerase, a RNA polymerase or reverse transcriptase. Polymerases used herein should have nucleic acid (e.g., DNA) strand displacement activity. “Strand displacement” describes the ability to displace downstream DNA encountered during synthesis. Examples of polymerases having DNA strand displacement activity that may be used as provided herein include, without limitation, phi29 (e.g., NEB #M0269) and Bst DNA polymerase (e.g., NEB #M0275). Phi29 polymerase is most active at moderate temperatures (e.g., 20-37° C.), while Bst polymerase is most active at elevated temperatures (e.g., 65° C.). In some embodiments, the polymerase is a DNA polymerase. In some embodiments, the polymerase is an RNA polymerase, reverse transcriptase or a polymerase engineered to incorporate non-natural nucleotides.

Imager Strand

In some embodiments, an imager strand is used to generate a detectable signal (a pulse). An imager strand is a single nucleic acid (e.g., DNA) strand that is linked to a detectable molecule (e.g., a molecule that emits a detectable signal, such as a fluorescent or chemiluminescent signal), referred to as a label. An imager strand comprises a sequence that is complementary to and can bind to a sequence on the product strand (specified by the template). In some embodiments, a imager strand has a length of 5-50 nucleotides. For example, a imager strand may have a length of 5-45, 5-40, 5-35, 5-30, 5-25, 5-20, 5-15, 5-10, 10, 40, 10-45, 10-40, 10-35, 10-30, 10-25, 10-20, 10-15, 15-50, 15-45, 15-40, 15-35, 15-30, 15-25, 15-20, 20-50, 20-45, 20-40, 20-35, 20-30, 20-25, 25-40, 25-35, 25-30, 30-50, 30-45, 30-40, 30-35 or 35-40 nucleotides. In some embodiments, a imager strand has a length of 5, 10, 15, 20, 25, 30, 35, 40, 45 or 50 nucleotides. In some embodiments, a imager strand has a length of 10, 11, 12, 13, 14, 15, 16, 17, 18, 19, 20, 21, 22, 23, 24 or 25 nucleotides. A imager strand, in some embodiments, is longer than 50 nucleotides, or shorter than 5 nucleotides.

In some embodiments, the label is a fluorophore. An imager strand linked to a fluorophore or other fluorescent/chemiluminescent molecule is referred to simply as a “fluorescent imager strand.” Examples of fluorophores that may be used herein include, without limitation, Hydroxycoumarin, methoxycoumarin, Alexa fluor, aminocoumarin, Cy2, FAM, Alexa fluor 405, Alexa fluor 488, Fluorescein FITC, Alexa fluor 430, Alexa fluor 532, HEX, Cy3, TRITC, Alexa fluor 546, Alexa fluor 555, R-phycoerythrin (PE), Rhodamine Red-X, Tamara, Cy3.5 581, Rox, Alexa fluor 568, Red 613, Texas Red, Alexa fluor 594, Alexa fluor 633, Allophycocyanin, Alexa fluor 647, Cy5, Alexa fluor 660, Cy5.5, TruRed, Alexa fluor 680, Cy7 and Cy7.5. Other fluorophores and molecules that emit a detectable signal are encompassed by the present disclosure.

In some embodiments, a template specifies more than one imager strand binding sequence on a product strand (each imager strand binding sequence having a different sequence relative to one another, thus, each “distinct” on the product strand). Thus, some compositions comprise more than one type of imager strand. “Types” of imager strands differ by their sequence composition (number, type and arrangement of nucleotides). Different types of imager strands may also have different types of labels. For example, one imager strand in a composition may have a red fluorophore, while another imager strand in the same composition may have a blue fluorophore (see, e.g., FIG. 20). Thus, a composition may comprise at least one, at least two or at least three different imager strands, each having a different “spectrally-distinct” label (e.g., red v. blue v. green).

Binding (stable binding) of an imager strand to a product strand results in emission (a “pulse”) of a fluorescent signal. In some embodiments, the duration of the fluorescent pulse is limited by photobleaching of the label associated with the imager strand. For example, a bound imager strand label may photobleach 0.01-100 seconds after binding. In some embodiments, the duration of the fluorescent pulse is controlled by subsequent binding of a quencher-labeled oligonucleotide that is complementary to a sequence adjacent to the imager binding site on the product strand (i.e., quencher binding site). In such a case, the duration of the pulse may be controlled in part by the rate of the multi-step addition of nucleotides comprising the adjacent quencher binding site to the product. For example, a quencher-labeled strand may bind 0.01-100 seconds after the imager strand binds. In some embodiments, the duration of the fluorescent pulse is controlled by subsequent degradation of the imager strand by an exonuclease (e.g., lambda exonuclease) that selectively degrades the imager strand upon binding to the template. For example, lambda exonuclease may degrade an imager strand 0.1-100 seconds after imager strand binding to the product strand. In some embodiments, the fluorescent signal of an imager strand lasts for ˜0.1-100 seconds, depending, in part, on the photostability of the imager strand label, the intensity of illumination during imaging, the number of nucleotides that must be added before quencher binding can occur, and the reaction conditions (e.g., buffer, temperature, dNTP concentration, oxygen concentration, concentration of quencher strand, concentration of exonuclease).

The amount of time between pulses may be controlled, in some embodiments, by varying the distance between imager strand binding sequences along the product or template, buffer conditions, temperature, and/or deoxynucleotides (dNTPs) concentrations in a reaction, as DNA polymerases, like most enzymes, are sensitive to many buffer conditions, including ionic strength, pH and types of metal ions present (e.g., sodium ions vs. magnesium ions). For example, increasing the temperature of a reaction using phi29 polymerase from 15° C. to 30° C. decreases dwell time. Likewise, increasing the temperature of a reaction using Bst DNA polymerase, large fragment from room temperature (˜25° C.) to 65° C. (the optimal temperature for polymerization by this enzyme) decreases dwell time. Thus, the temperature at which a reaction is performed may vary from, for example, 4° C. to 65° C. (e.g., 4° C., 25° C., 37° C., 42° C. or 65° C.). In some embodiments, a reaction is performed at room temperature, while in other embodiments, a reaction is performed at 37° C. As another example, increasing salt concentration (e.g., increasing [NaCl] from 40 mM to 200 mM) results in slower DNA polymerization and longer times between pulses (e.g., for Bst DNA polymerase, large fragment).

In some embodiments, the concentration of dNTPs in a reaction is 100 nM-100 μM. For example, the concentration of dNTPs in a reaction may be 100, 200, 300, 400, 500, 600, 700, 800, 900 or 1000 nM (μM). In some embodiments, the concentration of dNTPs in a reaction is 2.5 μM-100 μM. For example, the concentration of dNTPs in a reaction may be 2.5-75, 2.5-50, 2.5-25, 2.5-20, 2.5-5, 5-100, 5-75, 5-50, 5-25, 5-20, 10-100, 10-75, 10-50, 105-25, 10-20, 25-100, 25-75, 25-50, 50-100, 50-75 or 75-100 μM. In some embodiments, the concentration of dNTPs in a reaction is 0.1, 0.5, 1, 1.5, 2, 2.5, 5, 10, 15, 20, 25, 30, 35, 40, 45, 50, 55, 60, 65, 70, 75, 80, 85, 90, 90 or 100 μM. In some embodiments, the concentration of dNTPs in a reaction is 2.5 μM to 1 mM, or 2.5 μM to 10 mM. For example, the concentration of dNTPs in a reaction may be 0.5, 1 mM, 5 mM or 10 mM.

Labeled dNTPs

In some embodiments, the incorporation of one or more labeled species of nucleotide triphosphate monomer (for example, fluorescently labeled ribonucleoside triphosphates (NTPs), deoxyribonucleoside triphosphates (dNTPs), or a non-natural nucleic acid monomer) is used to generate a detectable signal (pulse). In some embodiments, the labeled oligonucleotide comprises both a fluorescent label and a quencher that is removed upon incorporation into the product strand (i.e., an internally quenched nucleotide), resulting in an enhancement of fluorescent signal upon incorporation. In some embodiments, the template comprises one or more contiguous nucleotide sequences (pulse zones) that template the addition of at least one fluorescent nucleotides. For example, a pulse zone may template the incorporation of 1-100 (e.g., 5, 10, 15, 20, 30, 40, 50, 60, 70, 80, 90 or 100) fluorescent nucleotides. In some embodiments, the pulse zones are separated by contiguous sequences that template the addition of only non-labeled nucleotides (non-pulse zones). In some embodiments, the duration of a pulse is determined by the amount of time required to incorporate all labeled nucleotides within a pulse zone. In some embodiments, the duration of a pulse is determined by the rate of photobleaching of a fluorescent label. In some embodiments, the duration of a pulse is determined by the rate of degradation of the product strand by an exonuclease. In some embodiments, the delay between fluorescent pulses is controlled by the number of nucleotides in a non-pulse zone. For example, 1-1000 (e.g., 10, 20, 30, 40, 50, 100, 200, 300, 400, 500, 600, 700, 800, 900, or 1000) non-labeled nucleotides may be incorporated between consecutive labeled nucleotides, resulting in a delay of ˜0.1-1000 seconds between pulses.

### Nucleic Acids

It should be understood that the nucleic acids of the present disclosure do not occur in nature. Thus, the nucleic acids may be referred to as “engineered nucleic acids.” An “engineered nucleic acid” is a nucleic acid (e.g., at least two nucleotides covalently linked together, and in some instances, containing phosphodiester bonds, referred to as a phosphodiester “backbone”) that does not occur in nature. Engineered nucleic acids include recombinant nucleic acids and synthetic nucleic acids. A “recombinant nucleic acid” is a molecule that is constructed by joining nucleic acids (e.g., isolated nucleic acids, synthetic nucleic acids or a combination thereof) and, in some embodiments, can replicate in a living cell. A “synthetic nucleic acid” is a molecule that is amplified or chemically, or by other means, synthesized. A synthetic nucleic acid includes those that are chemically modified, or otherwise modified, but can base pair with (also referred to as “binding to,” e.g., transiently or stably) naturally-occurring nucleic acid molecules. Recombinant and synthetic nucleic acids also include those molecules that result from the replication of either of the foregoing.

While an engineered nucleic acid, as a whole, is not naturally-occurring, it may include wild-type nucleotide sequences. In some embodiments, an engineered nucleic acid comprises nucleotide sequences obtained from different organisms (e.g., obtained from different species). For example, in some embodiments, an engineered nucleic acid includes a murine nucleotide sequence, a bacterial nucleotide sequence, a human nucleotide sequence, a viral nucleotide sequence, or a combination of any two or more of the foregoing sequences.

In some embodiments, an engineered nucleic acid of the present disclosure may comprise a backbone other than a phosphodiester backbone. For example, an engineered nucleic acid, in some embodiments, may comprise phosphoramide, phosphorothioate, phosphorodithioate, O-methylphophoroamidite linkages, peptide nucleic acids or a combination of any two or more of the foregoing linkages. An engineered nucleic acid may be single-stranded (ss) or double-stranded (ds), as specified, or an engineered nucleic acid may contain portions of both single-stranded and double-stranded sequence. In some embodiments, an engineered nucleic acid contains portions of triple-stranded sequence, or other non-Watson-Crick base pairing such as G-quartets, G-quadruplexes, and i-motifs. An engineered nucleic acid may comprise DNA (e.g., genomic DNA, cDNA or a combination of genomic DNA and cDNA), RNA or a hybrid molecule, for example, where the nucleic acid contains any combination of deoxyribonucleotides and ribonucleotides (e.g., artificial or natural), and any combination of two or more bases, including uracil, adenine, thymine, cytosine, guanine, inosine, xanthine, hypoxanthine, isocytosine and isoguanine.

Nucleic acids (e.g., circular template, circular leash, imager strands and/or primers) of the present disclosure may be produced using standard molecular biology methods (see, e.g., Green and Sambrook, Molecular Cloning, A Laboratory Manual, 2012, Cold Spring Harbor Press). In some embodiments, nucleic acids are produced using GIBSON ASSEMBLY® Cloning (see, e.g., Gibson, D.G. et al. Nature Methods, 343-345, 2009; and Gibson, D.G. et al. Nature Methods, 901-903, 2010, each of which is incorporated by reference herein). GIBSON ASSEMBLY® typically uses three enzymatic activities in a single-tube reaction: 5′ exonuclease, the 3′ extension activity of a DNA polymerase and DNA ligase activity. The 5′ exonuclease activity chews back the 5′ end sequences and exposes the complementary sequence for annealing. The polymerase activity then fills in the gaps on the annealed domains. A DNA ligase then seals the nick and covalently links the DNA fragments together. The overlapping sequence of adjoining fragments is much longer than those used in Golden Gate Assembly, and therefore results in a higher percentage of correct assemblies. Other methods of producing nucleic acids are known in the art and may be used in accordance with the present disclosure.

### Kinetic Encoding and Compact Barcoding

An individual single-molecule clock exhibits characteristic changes in fluorescence over time, with intensity spikes (“ON”) that occur amid a low-fluorescence background (“OFF”), with the duration between consecutive ON states, OFF states, or any combination thereof precisely controlled. Due to the use of a circular template, the series of ON and OFF states may occur and be observed several times in succession for greater confidence in the signal. By varying the characteristic dwell times in the ON and OFF states of a clock system (e.g., by varying the number of nucleotides between imager binding sites on the product, or the number of nucleotides between an imager binding site and a quencher binding site on the product), multiple clocks that are distinguishable by kinetics alone due to their well-separated dwell time distributions can be constructed. In a polymerase-based clock system, the mean dwell time between two events is linearly dependent on the length of the intervening template sequence, allowing the systematic design of multiple distinguishable clocks. For multiplexed detection, a single-molecule clock system may comprise a template that, though the action of a polymerase, generates an ordered series of ON and OFF states of defined length and other distinguishable characteristics (e.g., color of fluorescence) to create kinetic barcodes that can be read out in minutes. The number of possible distinguishable kinetic barcodes is dictated by the number of pulses per cycle encoded by the template; the number of distinguishable delays between consecutive pulses, OFF states, or any combination thereof; and the number of distinguishable labels (e.g., fluorophores of different emission or excitation wavelength). For example, a clock with four pulses, two distinguishable delay times, and three distinguishable labels can distinguish (2×3)⁴=1,296 targets. This high degree of multiplexing does not require any kind of strand exchange or spatially resolved (i.e. by direct imaging) encoding of information. In the embodiments discussed above, each barcode may be only tens of nanometers in size, making super-resolution imaging within intact cells and tissues a possibility.

### Rapid, One-Pot Multiplexing

Unlike existing approaches to multiplexing in fluorescence microscopy, barcoding with single-molecule clocks does not require multiple buffer exchanges, photobleaching steps, or ex situ imaging. Rather, the sample can be imaged directly in the presence of the mixture of clocks, because they all can be distinguished by a combination of dwell time and wavelength. This imaging may take place over minutes or tens of minutes, rather than the several hours or days required for competing approaches.

### Multiplexed Super-Resolution Microscopy of Cells and Tissues

Single-molecule clocks may be used, in some embodiments, for multiplexed super-resolution microscopy of cells and tissues. Kinetic barcodes constructed from single-molecule clocks can used to detect and spatially resolve thousands of distinct targets in a single imaging experiment lasting only 1-10 minutes. The target biomolecules may be proteins, nucleic acids, or any other biomolecule for which selective affinity reagents (e.g., antibodies or hybridization probes) are available and may be conjugated to appropriate barcode sequences. In one embodiment, a DNA-barcoded antibody library is constructed; that is, each antibody species is associated with a unique DNA barcode specifying an ordered combination of pulse delays and colors. Each barcode, and hence, each antibody, is identifiable based on the combination of clock probes that bind to it. Furthermore, because single pulses are detected and can be localized using various existing fitting algorithms, super-resolution microscopy is possible. This technique can be used, for instance, in the high-content imaging of heterogeneous tissues, such as tumors, where the expression levels and localization of biomarkers within the tissue may help to characterize and respond to drug resistance.

The present disclosure further provides embodiments encompassed by the following numbered paragraphs:
1. A kinetically encoded imaging system, comprising:

(a) an unpaired initiator nucleic acid comprising a 3′ nucleotide subdomain and a 5′ nucleotide subdomain;

(b) a template probe comprising (i) an unpaired 5′ toehold domain, (ii) a hairpin stem domain formed by base pairing between nucleotides located in a 5′ subdomain of the probe and nucleotides located in a 3′ subdomain of the probe, and a hairpin loop domain; and

(c) a primer.
2. The system of paragraph 1, wherein the primer is linked to a detectable molecule.
3. The system of paragraph 1, wherein the template probe is linked to a detectable molecule.
4. The system of paragraph 1, wherein the 3′ nucleotide subdomain of the initiator of (a) is complementary to and binds to the unpaired toehold domain of the probe of (b), and the 5′ nucleotide subdomain of the initiator of (a) is complementary to and binds to the 5′ subdomain of the probe of (b).
5. The system of paragraph 1 or 4, wherein the primer is complementary to and binds to the 3′ subdomain of the probe of (b).
6. The system of any one of paragraphs 1-5, wherein the template probe further comprises 3′ phosphate (PO₄²⁻) group.
7. The system of any one of paragraphs 1-6 further comprising a DNA polymerase.
8. The system of paragraph 7, wherein the DNA polymerase has strand displacement activity.
9. The system of paragraph 8, wherein the DNA polymerase is phi29.
10. The system of paragraph 8, wherein the DNA polymerase is Bst DNA polymerase, large fragment.
11. The system of any one of paragraphs 1-10, wherein the initiator nucleic acid has a length of 15-50 nucleotides.
12. The system of paragraph 11, wherein the initiator nucleic acid has a length of 20-30 nucleotides.
13. The system of paragraph 11 or 12, wherein the 3′ nucleotide subdomain of the initiator nucleic acid has a length of 5-15 nucleotides.
14. The system of any one of paragraphs 11-13, wherein the 5′ nucleotide subdomain of the initiator nucleic acid has a length of 10-20 nucleotides.
15. The system of any one of paragraphs 1-14, wherein the template probe has a length of 30-200 nucleotides.
16. The system of paragraph 15, wherein the template probe has a length of 30-50 nucleotides.
17. The system of any one of paragraphs 1-16,wherein the toehold domain has a length of 2-15 nucleotides.

18. The system of any one of paragraphs 1-17,wherein the hairpin stem domain has a length of 10-20 nucleotides.
19. The system of any one of paragraphs 1-18, wherein the hairpin loop domain has a length of 4-100 nucleotides.
20. The system of paragraph 19, wherein the hairpin loop domain has a length of 4-20 nucleotides.
21. The system of any one of paragraphs 1-20, wherein the primer has length of 10-20 nucleotides.
22. A kinetically encoded imaging method, comprising:

combining in reaction buffer
(a) an unpaired initiator nucleic acid comprising a 3′ nucleotide subdomain and a 5′ nucleotide subdomain, wherein the initiator nucleic acid is associated with a target of interest,
(b) a hairpin template probe comprising (i) an unpaired 5′ toehold domain, (ii) a hairpin stem domain formed by base pairing between nucleotides located in a 5′ subdomain of the probe and nucleotides located in a 3′ subdomain of the probe, and a hairpin loop domain,
(c) a primer linked to a detectable molecule,
(d) a DNA polymerase, and
(e) dNTPs, thereby forming a reaction mixture; and

incubating the reaction mixture under conditions that result in DNA polymerization.
23. The method of paragraph 22, wherein the 3′ nucleotide subdomain of the initiator of (a) is complementary to and binds to the unpaired toehold domain of the probe of (b), and the 5′ nucleotide subdomain of the initiator of (a) is complementary to and binds to the 5′ subdomain of the probe of (b).
24. The method of paragraph 22 or 23, wherein the primer is complementary to and binds to the 3′ subdomain of the probe of (b).
25. The method of any one of paragraphs 22-24 further comprising imaging the reaction mixture during the incubation step and identifying periods of time during which there is an increase in a level of fluorescence relative to a start time control level of fluorescence, thereby identifying dwell times.
26. The method of paragraph 25 further comprising identifying the presence or absence of a target of interest based on the dwell times.
27. The method of any one of paragraphs 22-26, wherein the initiator nucleic acid has a length of 15-50 nucleotides
28. The method of paragraph 27, wherein the initiator nucleic acid has a length of 20-30 nucleotides
29. The method of paragraph 27 or 28, wherein the 3′ nucleotide subdomain of the initiator nucleic acid has a length of 5-15 nucleotides.
30. The method of any one of paragraphs 27-29, wherein the 5′ nucleotide subdomain of the initiator nucleic acid has a length of 10-20 nucleotides.
31. The method of any one of paragraphs 22-30, wherein the template probe has a length of 30-200 nucleotides.
32. The method of paragraph 31, wherein the template probe has a length of 30-50 nucleotides.
33. The method of any one of paragraphs 22-32,wherein the toehold domain has a length of 5-15 nucleotides.
34. The method of any one of paragraphs 22-33,wherein the hairpin stem domain has a length of 10-20 nucleotides.
35. The method of any one of paragraphs 22-34, wherein the hairpin loop domain has a length of 4-100 nucleotides.
36. The method of paragraph 35, wherein the hairpin loop domain has a length of 4-20 nucleotides.
37. The method of any one of paragraphs 22-36, wherein the primer has length of 10-20 nucleotides.
38. The method of any one of paragraphs 22-37, wherein the template probe further comprises 3′ phosphate (PO₄²⁻) group.
39. The method of any one of paragraphs 22-37, wherein the DNA polymerase has strand displacement activity.
40. The method of paragraph 39, wherein the DNA polymerase is phi29.
41. The method of paragraph 39, wherein the DNA polymerase is Bst DNA polymerase, large fragment.
42. The method of any one of paragraphs 22-41, wherein the detectable molecule is a fluorophore.
43. The method of any one of paragraphs 22-42, wherein dNTPs are present at a concentration of 2.5 μM to 10 mM.
44. The method of paragraph 43, wherein dNTPs are present at a concentration of 100 μM.
45. A nucleic acid molecule comprising a 5′ paired domain, an internal unpaired domain, and a 3′ paired domain linked to a detectable molecule.
46. A kinetically encoded imaging system, comprising:

(a) a target nucleic acid;

(b) a 5′-phosphorylated nucleic acid probe linked to a 3′ detectable molecule; and

(c) a 5′-phosphate-specific exonuclease.
47. The system of paragraph 46, wherein the probe is complementary to and binds to the target.
48. A kinetically encoded imaging method, comprising:

combining in reaction buffer
(a) a target nucleic acid,
(b) a 5′-phosphorylated nucleic acid probe linked to a 3′ detectable molecule, and
(c) a 5′-phosphate-specific exonuclease; and

incubating the reaction mixture under conditions that result in exonuclease-mediated degradation of the probe.
49. A kinetically encoded imaging system, comprising:

(a) an unpaired initiator nucleic acid; and

(b) a first hairpin probe, a second hairpin probe, a third hairpin probe and a fourth hairpin probe, each hairpin probe comprising (i) an unpaired 5′ toehold domain, (ii) a hairpin stem domain formed by intramolecular base pairing between nucleotides located in a 5′ subdomain of the probe and nucleotides located in a 3′ subdomain of the probe, and a hairpin loop domain located between the 5′ subdomain and the 3′ subdomain, wherein the first hairpin probe is linked to a detectable molecule.
50. The system of paragraph 49, wherein

(i) the toehold domain and the 5′ subdomain of the first probe are complementary to and bind to the initiator nucleic acid;

(ii) the toehold domain and the 5′ subdomain of the second probe are complementary to and bind to the hairpin stem domain and the 3′ subdomain of the first probe bound to the initiator sequence;

(iii) the toehold domain and the 5′ subdomain of the third probe are complementary to and bind to the hairpin stem domain and the 3′ subdomain of the second probe bound to the first probe; and

(iv) the toehold domain and the 5′ subdomain of the fourth probe are complementary to and bind to the hairpin stem domain and the 3′ subdomain of the third probe bound to the second probe, and wherein the hairpin loop and 3′ subdomain of the fourth probe are complementary to and bind to the toehold domain and the 5′ subdomain of the first probe.
51. A kinetically encoded imaging method, comprising:

combining in reaction buffer
(a) an unpaired initiator nucleic acid, and
(b) a first hairpin probe, a second hairpin probe, a third hairpin probe and a fourth hairpin probe, each hairpin probe comprising (i) an unpaired 5′ toehold domain, (ii) a hairpin stem domain formed by intramolecular base pairing between nucleotides located in a 5′ subdomain of the probe and nucleotides located in a 3′ subdomain of the probe, and a hairpin loop domain located between the 5′ subdomain and the 3′ subdomain, wherein the first hairpin probe is linked to a detectable molecule; and

incubating the reaction mixture under conditions that result in DNA hybridization.
52. A composition, comprising:

(a) a circular nucleic acid template comprising a primer binding sequence and interlocked with a circular nucleic acid leash;

(b) a nucleic acid primer comprising a sequence complementary to the primer binding sequence; and

(c) a labeled nucleic acid imager strand.
53. The composition of paragraph 52, wherein the template comprises deoxyribonucleic acid (DNA).
54. The composition of paragraph 52, wherein the leash is a circular nucleic acid leash strand.
55. The composition of any one of paragraphs 52-54, wherein the template has a length of 50-1000 nucleotides.
56. The composition of paragraph 55, wherein the template has a length of 50-500 nucleotides.
57. The composition of any one of paragraphs 52-56, wherein the leash has a length of 50-1000 nucleotides.
58. The composition of any one of paragraphs 52-57, wherein the primer comprises DNA or RNA.
59. The composition of any one of paragraphs 52-58, wherein the primer has a length of 5-50 nucleotides.
60. The composition of any one of paragraphs 52-59, wherein the imager strand comprises DNA.
61. The composition of any one of paragraphs 52-60, wherein the imager strand has a length of 5-50 nucleotides.
62. The composition of any one of paragraphs 52-61, wherein the imager strand comprises a sequence complementary to sequence encoded by the primer binding sequence.
63. The composition of any one of paragraphs 52-61, wherein the imager strand comprises a sequence complementary to a sequence encoded by the template, other than the reverse complement of the primer binding sequence.
64. The composition of any one of paragraphs 52-63, wherein the label is a fluorescent label.
65. The composition of any one of paragraphs 52-64, wherein the imager strand is bound to a quencher strand comprising a quencher molecule and wherein the quencher strand is shorter than the imager strand.
66. The composition of any one of paragraphs 52-65, wherein the fluorescent label is located at the 3′ end of the imager strand and the quencher molecule is located at the 5′ end of the quencher strand.
67. The composition of any one of paragraphs 52-66, wherein the composition further comprises an endonuclease.
68. The composition of any one of paragraphs 52-67 further comprising a polymerase selected from a DNA polymerase, a RNA polymerase and reverse transcriptase.
69. The composition of paragraph 68, wherein the polymerase has strand displacement activity.
70. The composition of paragraph 69, wherein the polymerase is a DNA polymerase.
71. The composition of paragraph 70, wherein the DNA polymerase is phi29 or Bst DNA polymerase, large fragment.
72. The composition of any one of paragraphs 52-71 further comprising at least one other labeled imager strand.
73. The composition of paragraph 72, wherein each of the other labeled imager strands comprises a sequence complementary to a distinct sequence encoded by the template.
74. The composition of paragraph 73, wherein the distinct sequences encoded by the template are separated from each by at least 10 nucleotides.
75. The composition of any one of paragraphs 72-74, wherein each of the imager strands comprises a spectrally-distinct fluorophore.
76. The composition of any one of paragraphs 52-75, wherein the circular nucleic acid leash is bound to an affinity tag that binds to a target of interest.
77. The composition of paragraph 76, wherein the target of interest is a protein or a nucleic acid.
78. A method, comprising:

combining in reaction buffer
(a) a circular nucleic acid template comprising a primer binding sequence and interlocked with a circular nucleic acid leash,
(b) a nucleic acid primer comprising a sequence complementary to the primer binding sequence,
(c) a labeled nucleic acid imager strand,
(d) a polymerase, and
(e) dNTPs or NTPs, thereby forming a reaction mixture; and

incubating the reaction mixture under conditions that result in nucleic acid polymerization and nucleic acid hybridization.
79. The method of paragraph 78, wherein the template comprises deoxyribonucleic acid (DNA).
80. The method of paragraph 78 or 79, wherein the template has a length of 50-1000 nucleotides.
81. The method of paragraph 80, wherein the template has a length of 50-500 nucleotides.
82. The method of any one of paragraphs 78-81, wherein the leash has a length of 50-1000 nucleotides.
83. The method of any one of paragraphs 78-82, wherein the primer comprises DNA.
84. The method of any one of paragraphs 78-83, wherein the primer has a length of 5-50 nucleotides.
85. The method of any one of paragraphs 78-84, wherein the imager strand comprises DNA.
86. The method of any one of paragraphs 78-85, wherein the imager strand has a length of 5-50 nucleotides.
87. The method of any one of paragraphs 78-86, wherein the imager strand comprises a sequence complementary to a distinct sequence encoded by the primer binding sequence.
88. The method of any one of paragraphs 78-86, wherein the imager strand comprises a sequence complementary to a distinct sequence encoded by the template, other than the reverse complement of the primer binding sequence.
89. The method of any one of paragraphs 78-88, wherein the label is a fluorescent label.
90. The method of any one of paragraphs 78-89, wherein the imager strand is bound to a quencher strand comprising a quencher molecule and wherein the quencher strand is shorter than the imager strand.
91. The method of any one of paragraphs 78-90, wherein the fluorescent label is located at the 3′ end of the imager strand and the quencher molecule is located at the 5′ end of the quencher strand.
92. The method of any one of paragraphs 78-91, wherein the composition further comprises an endonuclease.
93. The method of any one of paragraphs 78-92 wherein the polymerase is selected from a DNA polymerase, a RNB polymerase and reverse transcriptase.
94. The method of paragraph 93, wherein the polymerase has strand displacement activity.
95. The method of paragraph 94, wherein the polymerase is a DNA polymerase.
96. The method of paragraph 95, wherein the DNA polymerase is phi29 or Bst DNA polymerase, large fragment.
97. The method of any one of paragraphs 78-96 further comprising at least one other labeled imager strand.
98. The method of paragraph 97, wherein each of the other labeled imager strands comprises a sequence complementary to a distinct sequence encoded by the template.
99. The method of paragraph 98, wherein the distinct sequences encoded by the template are separated from each by at least 10 nucleotides.
100. The method of any one of paragraphs 97-99, wherein each of the imager strands comprises a spectrally-distinct fluorophore.
101. The method of any one of paragraphs 78-100, wherein the circular nucleic acid leash is bound to an affinity tag that binds to a target of interest.
102. The method of paragraph 101, wherein the target of interest is a protein or a nucleic acid.
103. The method of any one of paragraphs 78-102 further comprising imaging the reaction mixture during the incubation step and identifying periods of time during which there is an increase in a level of fluorescence relative to a start time control level of fluorescence, thereby identifying dwell times.
104. The method of paragraph 103 further comprising identifying the presence or absence of a target of interest based on the a pattern of fluorescence.
105. The method of any one of paragraphs 78-104, wherein dNTPs are present at a concentration of 2.5 μM to 10 mM.
106. The method of paragraph 105, wherein dNTPs are present at a concentration of 100 μM.
107. A composition, comprising:

(a) a circular nucleic acid template comprising a primer binding sequence and interlocked with a circular nucleic acid leash;

(b) a primer comprising a sequence complementary to the primer binding sequence; and

(c) a mixture of deoxynucleoside triphosphates (dNTPs) comprising subsets of dATPs, dTTPs, dCTPs and dGTPs, wherein dNTPs of at least one of the subsets comprise a label; or a mixture of nucleoside triphosphates (NTPs) comprising subsets of ATPs, TTPs, CTPs and GTPs, wherein NTPs of at least one of the subsets comprise a label.
108. The composition of paragraph 107, wherein the template comprises deoxyribonucleic acid (DNA).
109. The composition of paragraph 107 or 108, wherein the template has a length of 50-1000 nucleotides.
110. The composition of paragraph 109, wherein the template has a length of 50-500 nucleotides.
111. The composition of any one of paragraphs 107-110, wherein the leash has a length of 50-1000 nucleotides.
112. The composition of any one of paragraphs 107-111, wherein the primer comprises DNA.
113. The composition of any one of paragraphs 107-112, wherein the primer has a length of 5-50 nucleotides.
114. The composition of any one of paragraphs 107-113, wherein the label is a fluorescent label.
115. The composition of any one of paragraphs 107-114, wherein the imager strand is bound to a quencher strand comprising a quencher molecule and wherein the quencher strand is shorter than the imager strand.
116. The composition of any one of paragraphs 107-115, wherein the fluorescent label is located at the 3′ end of the imager strand and the quencher molecule is located at the 5′ end of the quencher strand.
117. The composition of any one of paragraphs 107-116, wherein the composition further comprises an endonuclease.
118. The composition of any one of paragraphs 107-117 further comprising a polymerase selected from a DNA polymerase, a RNA polymerase and reverse transcriptase.
119. The composition of paragraph 118, wherein the polymerase has strand displacement activity.
120. The composition of paragraph 119, wherein the polymerase is a DNA polymerase.
121. The composition of paragraph 120, wherein the DNA polymerase is phi29 or Bst DNA polymerase, large fragment.
122. The composition of any one of paragraphs 107-121, wherein the circular nucleic acid leash is bound to an affinity tag that binds to a target of interest.
123. The composition of paragraph 122, wherein the target of interest is a protein or a nucleic acid.
124. A method, comprising:

combining in reaction buffer
(a) a circular nucleic acid template comprising a primer binding sequence and interlocked with a circular nucleic acid leash,
(b) a primer comprising a sequence complementary to the primer binding sequence,
(c) a mixture of dNTPs comprising subsets of dATPs, dTTPs, dCTPs and dGTPs, wherein dNTPs of at least one of the subsets comprise a label, or mixture of NTPs comprising subsets of ATPs, TTPs, CTPs and GTPs, wherein NTPs of at least one of the subsets comprise a label, and
(d) a polymerase; and

incubating the reaction mixture under conditions that result in nucleic acid polymerization and nucleic acid hybridization.
125. The method of paragraph 124, wherein the template comprises deoxyribonucleic acid (DNA).
126. The method of paragraph 124 or 125, wherein the template has a length of 50-1000 nucleotides.
127. The method of paragraph 126, wherein the template has a length of 50-500 nucleotides.
128. The method of any one of paragraphs 124-127, wherein the leash has a length of 50-1000 nucleotides.
129. The method of any one of clams 124-128, wherein the primer comprises DNA.
130. The method of any one of clams 124-129, wherein the primer has a length of 5-50 nucleotides.
131. The method of any one of clams 124-130, wherein the label is a fluorescent label.
132. The method of any one of paragraphs 124-131, wherein the imager strand is bound to a quencher strand comprising a quencher molecule and wherein the quencher strand is shorter than the imager strand.
133. The method of any one of paragraphs 124-132, wherein the fluorescent label is located at the 3′ end of the imager strand and the quencher molecule is located at the 5′ end of the quencher strand.
134. The method of any one of paragraphs 124-133, wherein the composition further comprises an endonuclease.
135. The method of any one of paragraphs 124-134 wherein the polymerase is selected from a DNA polymerase, a RNA polymerase and reverse transcriptase.
136. The method of paragraph 135, wherein the polymerase has strand displacement activity.
137. The method of paragraph 136, wherein the polymerase is a DNA polymerase.
138. The method of paragraph 137, wherein the DNA polymerase is phi29 or Bst DNA polymerase, large fragment.
139. The method of any one of paragraphs 124-138, wherein the circular nucleic acid leash is bound to an affinity tag that binds to a target of interest.
140. The method of paragraph 139, wherein the target of interest is a protein or a nucleic acid.
141. The method of any one of paragraphs 124-140 further comprising imaging the reaction mixture during the incubation step and identifying periods of time during which there is an increase in a level of fluorescence relative to a start time control level of fluorescence, thereby identifying dwell times.
142. The method of paragraph 141 further comprising identifying the presence or absence of a target of interest based on the a pattern of fluorescence.
143. The method of any one of paragraphs 124-142, wherein dNTPs are present at a concentration of 2.5 μM to 10 mM.
144. The method of paragraph 143, wherein dNTPs are present at a concentration of 100 μM.
145. A composition, comprising:

(a) a circular nucleic acid template comprising a primer binding sequence and interlocked with a circular nucleic acid leash;

(b) a nucleic acid primer comprising a sequence complementary to the primer binding sequence; and

(c) a labeled nucleic acid imager strand bound to a quencher strand comprising a quencher molecule, wherein the quencher strand is shorter than the imager strand.
146. The composition of paragraph 145, wherein the fluorescent label is located at the 3′ end of the imager strand and the quencher molecule is located at the 5′ end of the quencher strand.
147. The composition of paragraph 145 or 146, wherein the composition further comprises an endonuclease.
148. A method, comprising:

combining in reaction buffer
(a) a circular nucleic acid template comprising a primer binding sequence and interlocked with a circular nucleic acid leash,
(b) a nucleic acid primer comprising a sequence complementary to the primer binding sequence,
(c) a labeled nucleic acid imager strand bound to a quencher strand comprising a quencher molecule, wherein the quencher strand is shorter than the imager strand,
(d) a polymerase, and
(e) dNTPs or NTPs, thereby forming a reaction mixture; and

incubating the reaction mixture under conditions that result in nucleic acid polymerization and nucleic acid hybridization.
149. The method of paragraph 148, wherein the fluorescent label is located at the 3′ end of the imager strand and the quencher molecule is located at the 5′ end of the quencher strand.
150. The method of paragraph 148 or 149, wherein the reaction buffer further comprises an endonuclease.

The present disclosure is further illustrated by the following Examples, which in no way should be construed as further limiting. The entire contents of all of the references (including literature references, issued patents, published patent applications, and co pending patent applications) cited throughout this application are hereby expressly incorporated by reference, in particular for the teachings that are referenced herein.

### EXAMPLES

### Single-Molecule Timers

### Example 1

As the number of irreversible steps between binding and dissociation increases, two trends are expected: (1) Δ̅t̅ should increase in proportion to template length, and (2) individual dwell times should become more narrowly distributed with respect to the mean value, or more deterministic. To test these predictions, a set of templates were constructed with identical toehold and stem domains but with varying length of the terminal loop, resulting in total template lengths ranging from 41 to 153 nucleotides, including the ˜14 nucleotide primer footprint that does not serve as a template for polymerization (FIG. 4A-4D). For longer loops, short hairpins were intentionally introduced to prevent unwanted base-pairing between the stem and terminal loop, and to reduce the entropic penalty for loop closure. The four template designs were assayed for single-molecule timer behavior under identical conditions, in the presence of a saturating concentration (100 μM) of dNTPs. All of the timers exhibited repeated bursts of fluorescence with a precisely determined Δt that increased with template size (FIG. 4A-4D). Several of the Δt distributions are well-separated from one another (FIG. 4E), making it possible in principle to distinguish between templates from the duration of only one binding event. Fitting the main peak of each dwell time histogram to a gamma distribution yields estimates of Δ̅t̅ that depend linearly on the length of the template (FIG. 4F), providing further evidence that polymerization is the primary rate-limiting process prior to complex dissociation. The slope of a linear fit yields an estimated elongation rate of ˜8.3 nt s⁻¹, considerably slower than the maximum elongation rate of 191.2 nt s⁻¹ reported for this polymerase at 65° C.¹³ but much faster than the maximum rate of 0.047-0.24 nt s⁻¹ reported at room temperature using coumarin-modified dNTPs¹² (all experiments in the present study were performed at room temperature using unmodified dNTPs). In addition to Δ̅t̅, fitting of a gamma distribution to the experimental Δt histograms yields estimates of Nₐₚₚ, the apparent number of irreversible steps between binding and dissociation, and Δ̅t̅ₛ̅ₜ̅ₑ̅ₚ̅, the apparent average time for each step to occur. As expected, Nₐₚₚ increases linearly with the length of the template (data not shown); equivalently, the randomness parameter

$$r = \frac{\overline{{( Δ t )}^{2}} - {\overline{Δ t}}^{2}}{{\overline{Δ t}}^{2}} = \frac{1}{{N}_{app}}$$

is inversely proportional to template length, indicating more deterministic behavior as the number of steps increases. However, contrary to naïve predictions, Nₐₚₚ is not equal to the number of nucleotides added, but is related by a proportionality constant of ˜0.43, suggesting that not all nucleotide addition steps are equally rate-limiting. On the other hand, Δ̅t̅ₛ̅ₜ̅ₑ̅ₚ̅ is relatively constant across all studied template lengths (Supplementary Fig. X), consistent with expectations.

### Example 2

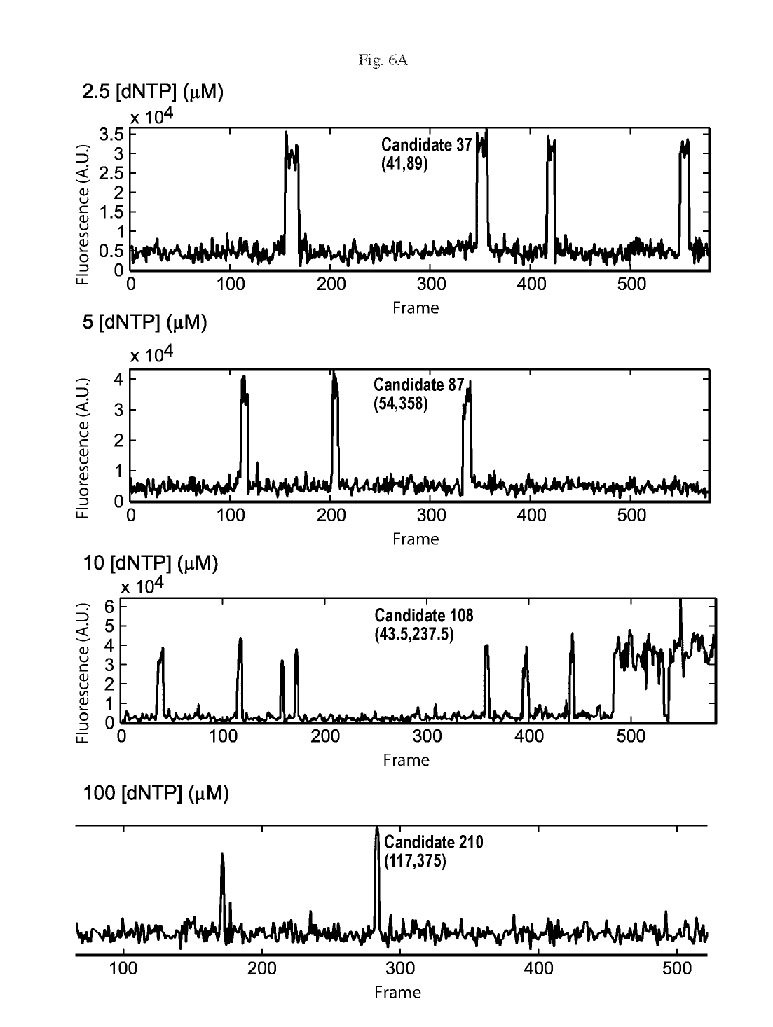

This Example provides data for characterizing parameters for gamma fit versus template length. When the gamma distribution function is fit to the experimental dwell time distributions (FIG. 4E), two parameters may be extracted: the apparent number of steps (Nsteps) and the apparent time required for each step. FIG. 5A shows that, as template length increases, the number of steps increased linearly Consequently, as the template length is increased, the width (or standard deviation) of the dwell time distribution becomes smaller relative to the mean. However, the observed slope of a linear fit to the data is significantly less than the theoretically predicted value of 1. The effective number of rate-limiting steps in the polymerization process is lower than the actual number of nucleotides added to the template. This results in dwell time distributions that are slightly broader than one would predict using the assumption that every nucleotide addition contributes equally to the overall dwell time. FIG. 5B shows that, as template length increases, the step time remains constant.

### Example 3

This Examples provides data for characterizing variation of the dwell time of single-molecule timers. Using a single template design (41 nucleotides), it is possible to vary the dwell time of single-molecule timers by varying the concentration of free nucleotides (dNTPs). This demonstrates that it is primarily the rate of DNA polymerization that controls the duration of timer binding events. FIG. 6A presents representative fluorescence time traces of timers operating with the 41-nucleotide template but in the presence of 2.5, 5, 10, or 100 μM dNTPs. In the 10 μM dNTP condition, the final 15% of the observation time shows an example of a longer dwell time. FIG. 6B depicts the dependence of the mean dwell time on dNTP concentration. The line represents a nonlinear least-squares fit of a Michaelis-Menten saturation curve to the data, resulting in an estimated K_{M} of 12.6 +/−2.4 μM and τₘᵢₙ of 2.1 +/−0.3 s (95% confidence bounds from fit).

### Example 4

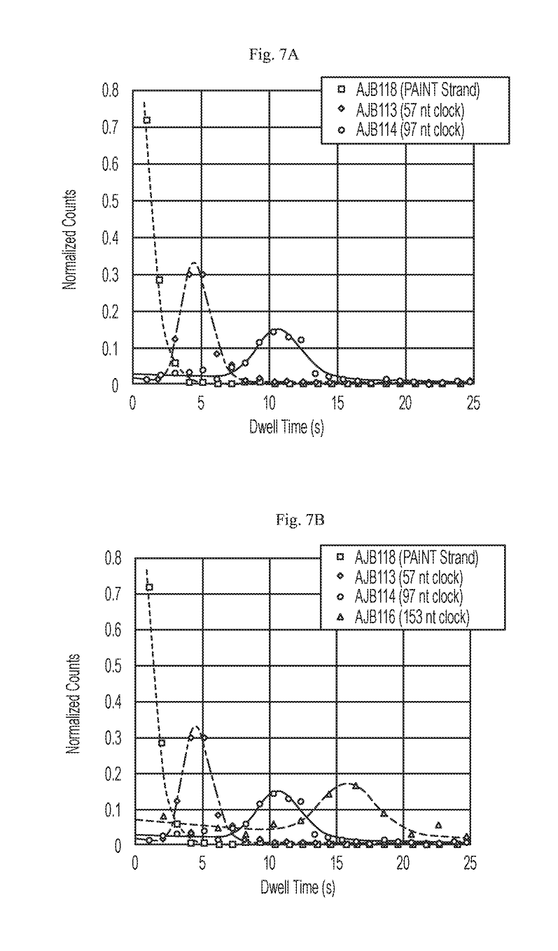

This Example provides data from a kinetic barcoding experiment using polymerase-based single-molecule timers. Multiple timers were used in the same experiment to construct barcodes for multiplexed imaging. In such a scheme, each barcode includes one or more initiator sequences, each of which binds to a specific timer. Each barcode's identity is determined by measuring the kinetics of timer binding to that barcode; the presence or absence of each timer lifetime is interpreted as a binary “bit” (1 or 0). Because the distributions of dwell times used for different “bits” do not overlap substantially, the presence or absence of each initiator sequence in the barcode can be determined with high confidence, particularly if many timer binding events are observed for each barcode. Two potential schemes for kinetic barcoding are shown. FIG. 7A depicts a 3-bit barcoding scheme that utilizes a DNA-PAINT probe with exponential dissociation kinetics, and two single-molecule timers with gamma-distributed kinetics. The graph in FIG. 7A shows experimental data for the PAINT probe and two single-molecule timers (with 57 and 97 nucleotide templates). For a given barcode sequence, the presence or absence of each dwell time may be read as a series of binary digits. For instance, if a DNA barcode sequence contains binding sites for the PAINT probe and both timers, all 3 dwell times will be detected, and the readout would be “111”. If only the initiator sequence for the 57-nucleotide timer were present in the barcode, the readout would be “010,” and so on. The number of distinct barcodes that can be distinguished is 2̂(N_{dt}*N_{colors})−1, where N_{dt} is the number of distinguishable dwell times and N_{colors} is the number of distinguishable colors. Using a 3-bit kinetic barcode with 1 color, 7 distinct barcodes can be resolved, with 2 colors, 63 distinct barcodes can be resolved, and with 3 colors, 511 distinct barcodes can be resolved (511 distinct molecular species can be imaged in a single experiment). FIG. 7B depicts a 4-bit barcode scheme. It is almost identical to the 3-bit barcode scheme, except with the addition of one extra timer that uses a 153-nucleotide template. In this scheme, a 1-color detection protocol permits 15 barcodes to be resolved; a 2-color detection protocol permits 255 barcodes to be resolved, and a 3-color detection protocol permits up to 4095 different barcodes (unique molecular targets) to be resolved. In practice, such barcoding schemes may involve covalently or noncovalently attaching a DNA molecule containing the appropriate combination of initiator sequences to targeting reagents such as antibodies. Then, the position and identity of each antibody can be determined by reading out the barcodes through imaging with single-molecule timers.

### Example 5

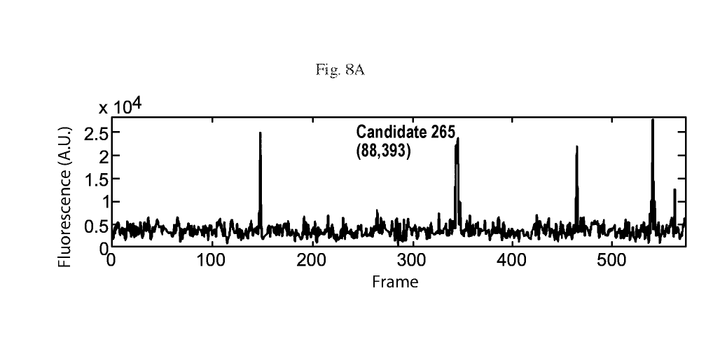

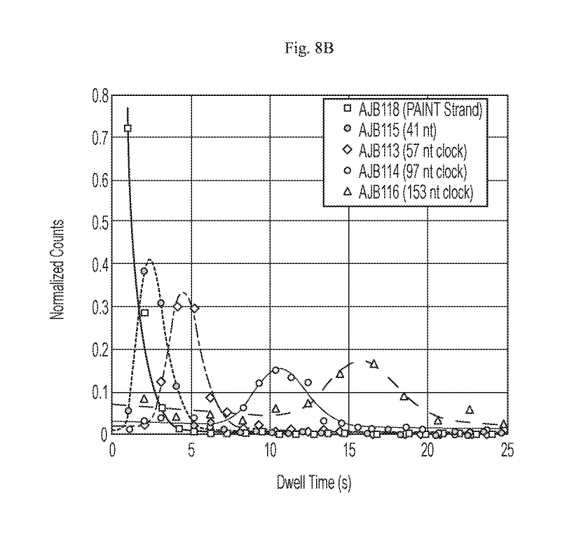

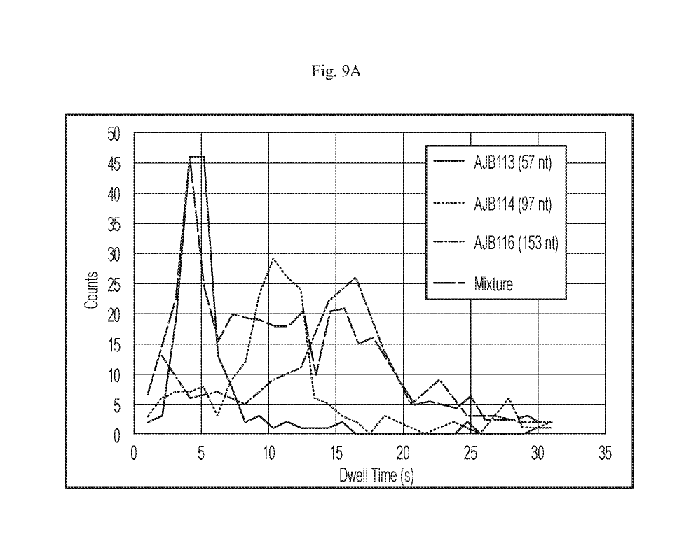

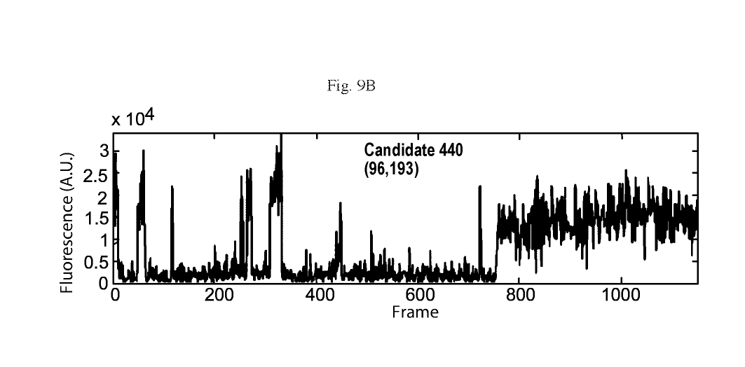

This Example shows that a short DNA-PAINT probe (Jungmann R et al. Nature Methods, 2014, 11, 313-318) may be used alongside single-molecule clocks to provide another well-resolved dwell time distribution (“bit”) in a kinetic barcode. A representative fluorescence vs. time trace is shown (FIG. 8A) along with the exponential distribution of dwell times in comparison to the gamma distributions of various timer (clock) probes (FIG. 8B).

### Example 6

This Example provides data representative of the simultaneous use of multiple timers. The mixture gives rise to a dwell time distribution that resembles the sum of the three underlying timer distributions. A representative single-molecule fluorescence trace is also shown. Dwell time distributions focus on binding events that last 30 seconds or less. There were not enough binding events per trace to classify each barcode, so it was necessary to increase Cy5 primer concentration, add quencher-labeled protector strand, and increase acquisition time.

### Example 7

This Example provides data of dwell time distributions with binding events lasting longer than 30 seconds. Rarely, binding events can have much longer dwell times. This gives rise to a background that appears exponential, rather than gamma-distributed. This portion of the dwell time distributions is shown above for three templates: 41 nt (FIG. 10A), 57 nt (FIG. 10B), and 97 nt (FIG. 10C). Representative traces are shown below. The relative abundance of these background binding events is somewhat higher for longer template constructs. For instance, it accounts for only ˜20% of observed binding events for the shortest template (41 nt; FIG. 10A), but ˜50% of binding events for the 57 (FIG. 10B) and 97 (FIG. 10C) nucleotide constructs. This may be mostly due to lower frequency of timer binding events for the longer constructs, rather than an increased frequency of background binding events.

### Example 8

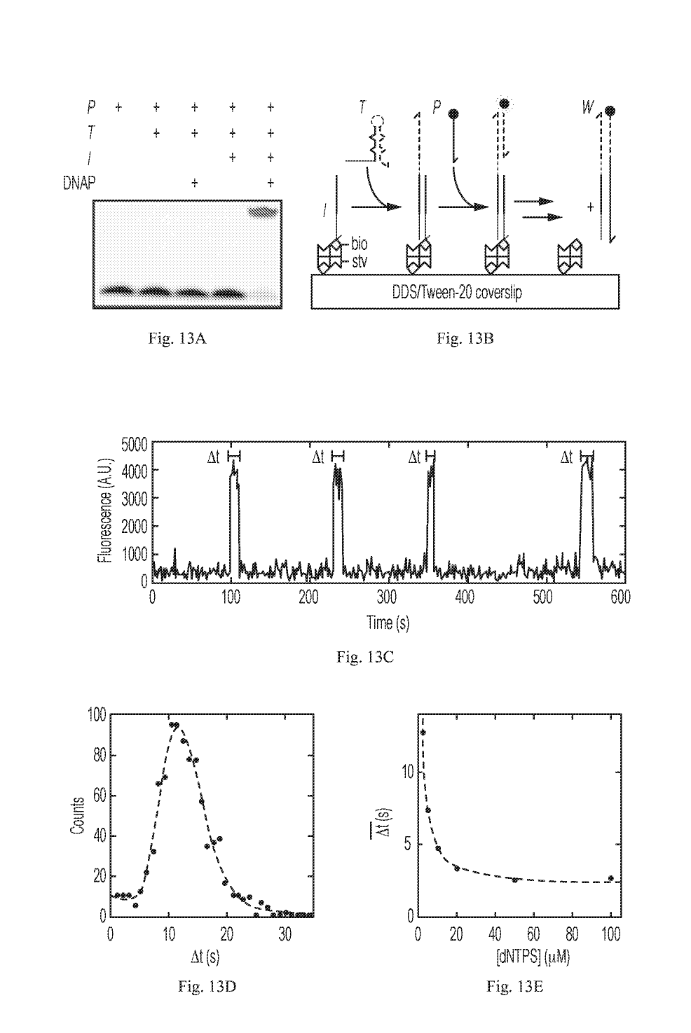

This Example provides additional data from a kinetic barcoding experiment using polymerase-based single-molecule timers. For a single-molecule timer of the type described in FIG. 3A to function, the following conditions should be met: (1) polymerization begins only upon binding of the initiator I, with minimal leakage; (2) DNAP initiates polymerization almost immediately after fluorescent primer P binds to T; and (3) elongation must be slow relative to the initiation of polymerization, enough to control the time delay between P binding and W dissociation. To address criterion (1), polyacrylamide gel electrophoresis was used to first demonstrated that a fluorescent primer is only extended in the presence of I, T, and DNAP (FIG. 13A). To address (2) and (3), a very high concentration of DNAP (˜1.6 μM) was used, and using single-molecule measurements confirmed that the rate of W dissociation is controlled by dNTP concentration (see below). Ensemble denaturing PAGE experiments were also performed to confirm that polymerase binding is fast relative to polymerization (data not shown).

To demonstrate deterministic timer behavior at the single-molecule level, a total internal reflection fluorescence (TIRF) assay was used in which biotinylated initiator strand I was immobilized on a passivated coverslip (FIG. 13B). In this experimental scheme, repeated cycles of increased and decreased fluorescence intensity are expected to occur at the position of each copy of I, with the duration of each burst controlled by polymerase activity. Such repeated fluorescence bursts are indeed observed (FIG. 13C), and as expected, exhibit a quite narrow, non-exponential distribution of dwell times (Δt) in the fluorescence state (FIG. 13D). Fitting the Δt histogram for a to a gamma probability distribution yields a mean dwell time (Δt) of 12.7+/−0.2 s for a 41-nucleotide template (T41). The rate of photobleaching (determined in absence of dNTPs) is much slower than that of polymerization-induced dissociation (data not shown). Consistent with the hypothesis that dwell time is predominantly controlled by the rate of nucleotide addition, (Δt) is strongly dependent on the concentration of dNTPs (FIG. 13E), exhibiting saturation behavior with an apparent K_{M} of 12.6+/−2.4 μM with respect to dNTPs.

FIG. 13A shows a denaturing polyacrylamide gel showing the DNAP-catalyzed extension of a Cy5-labeled primer (P) in the presence of a 41-nucleotide (nt) loop template, T₄₁. Extension of the Cy5-labeled primer occurs efficiently only in the presence of T₄₁, initiator (I), and DNAP. No detectable leakage occurs in absence of I. Incubation time: 5 min. FIG. 13B depicts a surface-based assay of single-molecule timer performance using total internal reflection fluorescence (TIRF) microscopy. Each surface-anchored copy of I undergoes repeated cycles of T₄₁ binding, P binding, extension by DNAP, and displacement of W. Localized fluorescence is only visible between binding of P and the dissociation of W. FIG. 13C shows a representative single-molecule fluorescence trajectory showing four consecutive timer binding cycles in the same location on a coverslip using T₄₁. FIG. 13D shows a dwell time distribution for T₄₁ in the presence of 2.5 μM dNTPs (n=1000 binding events). The red line represents a nonlinear least-squares fit of a gamma probability distribution to the data, yielding an estimated mean dwell time Δ̅t̅ of 12.7+/−0.2 s (95% confidence bounds from fit). FIG. 13E shows dependence of the mean dwell time on dNTP concentration. The line represents a nonlinear least-squares fit of a Michaelis-Menten saturation curve to the data, resulting in an estimated K_{M} of 12.6+/−2.4 μM and τₘᵢₙ of 2.1 +/−0.3 s (95% confidence bounds from fit).

### Example 9

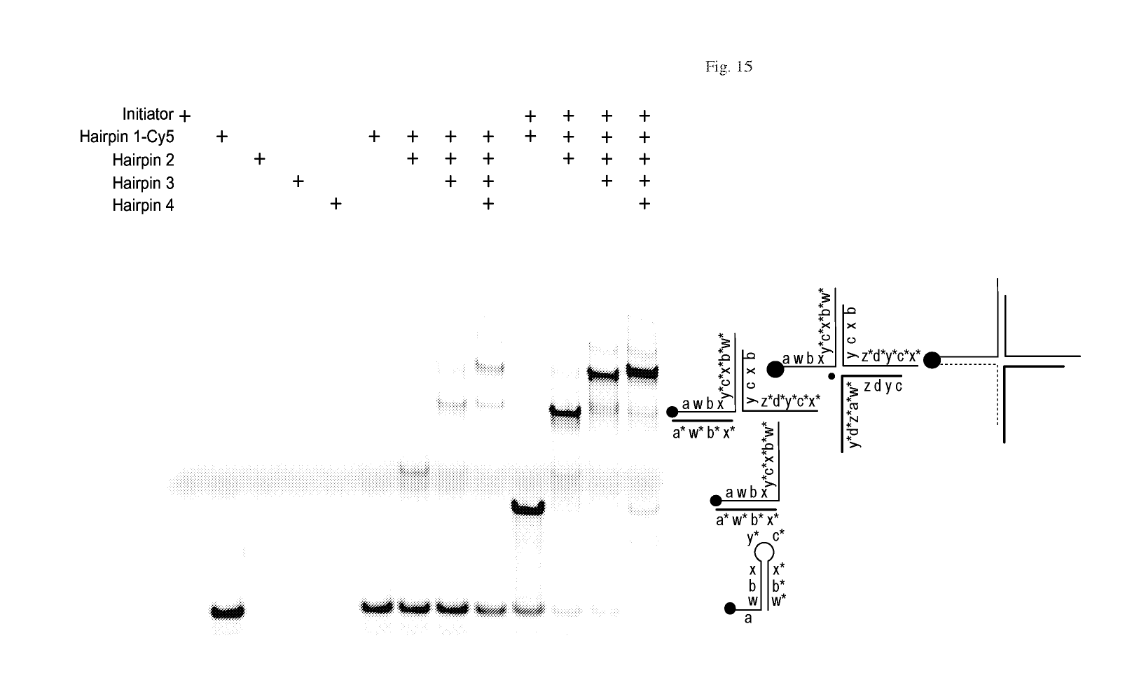

This Example provides additional data from a kinetic barcoding experiment using polymerase-based single-molecule timers. FIG. 15 shows a fluorescence scan of a native polyacrylamide gel electrophoresis experiment demonstrating that the four-way junction (waste) complex forms upon the addition of all four hairpin template probes and the initiator (target) nucleic acid. The band patterns indicate that some assembly of the hairpins occurs in absence of the initiator (“leakage”), but that such leakage is relatively minimal. Thus, in most cases, the hairpins will only proceed through the pathway once exposed to the initiator (target) nucleic acid.

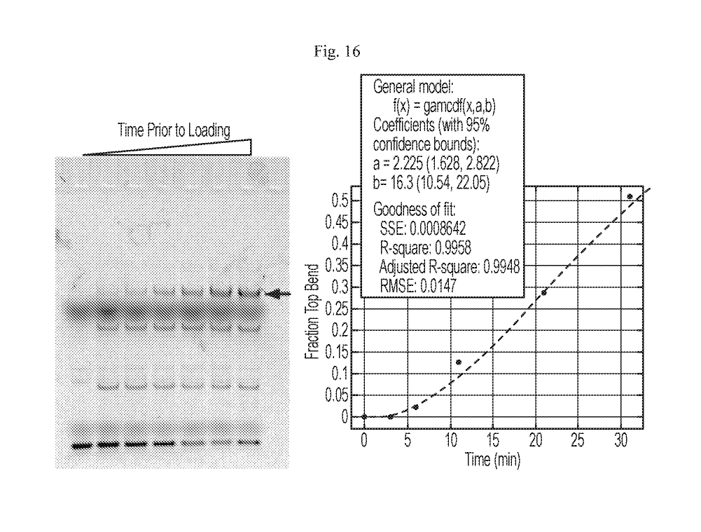

FIG. 16 shows a fluorescence scan of a native polyacrylamide gel electrophoresis experiment in which the formation of complexes from the hairpin template probes is monitored over time. The reaction was allowed to proceed in a test tube for varying amounts of time before loading onto a polyacrylamide gel and applying voltage to start electrophoresis. When the fluorescence of the top band was quantified as a function of time, a distinct time delay prior to product formation was evident (see graph on right). Fitting of a gamma cumulative distribution function to this data resulted in an estimate of the shape parameter of 2.225, suggesting that the effective number of rate-limiting steps in this hybridization cascade was only ˜2.2.

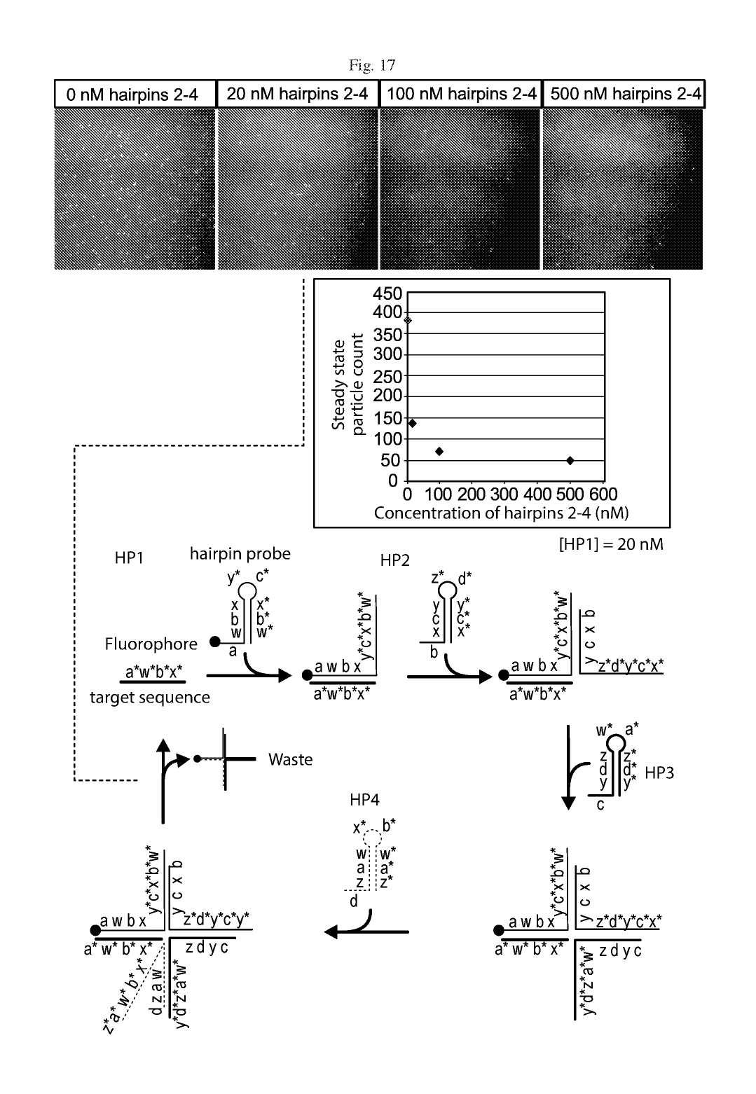

FIG. 17 shows data from internal reflection fluorescence (TIRF) microscopy at varying hairpin probe concentrations. When the initiator (target) nucleic acid was immobilized on a glass coverslip surface, the binding of the first, fluorescent hairpin (HP1, red) was detected at the level of single molecules using total TIRF microscopy. When the remaining 3 hairpin probes were added at varying concentrations (0-500 nanomolar, nM) while keeping the first hairpin probe's concentration at a constant 20 nM, the steady-state number of fluorescent spots visible per field of view decreased with increasing hairpin concentration. This is consistent with the expectation that hairpins 2-4 accelerate the dissociation of the fluorescent DNA molecule from the surface by proceeding through the indicated hybridization cascade.

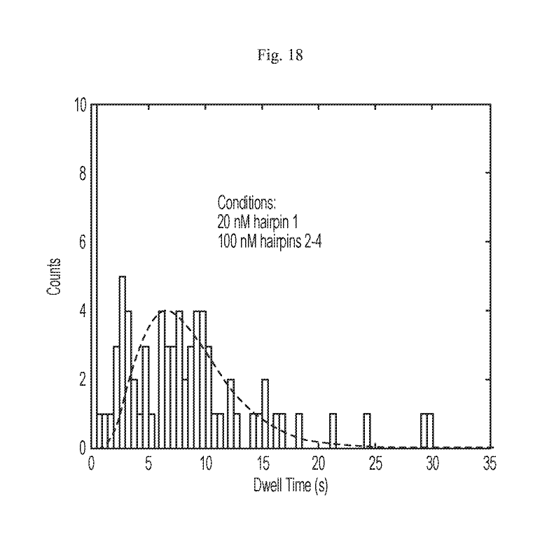

FIG. 18 shows a histogram of dwell times in the fluorescent state for a 4-step hybridization cascade along with a gamma distribution for a process with 4 steps (shape parameter=4) with a mean lifetime of 2 s per step. The histogram is largely consistent with a gamma distribution, but is not as sharp as the dwell time distribution seen for DNA polymerase-based timers. This is expected given the smaller number of steps in the hybridization cascade compared to the polymerase system.

### Single-Molecule Clocks

### Example 10

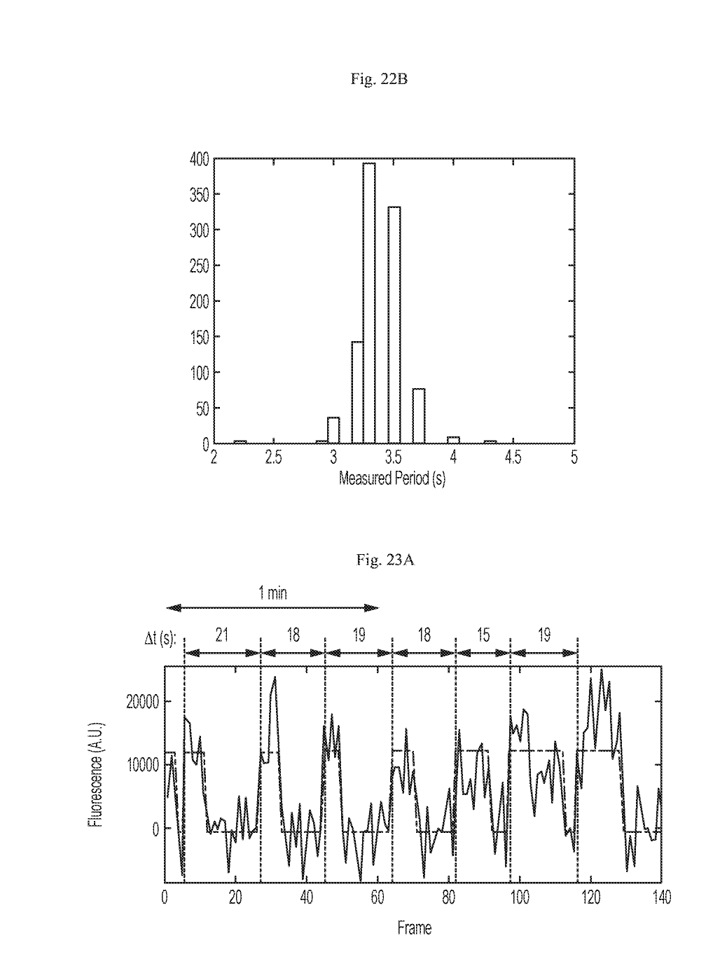

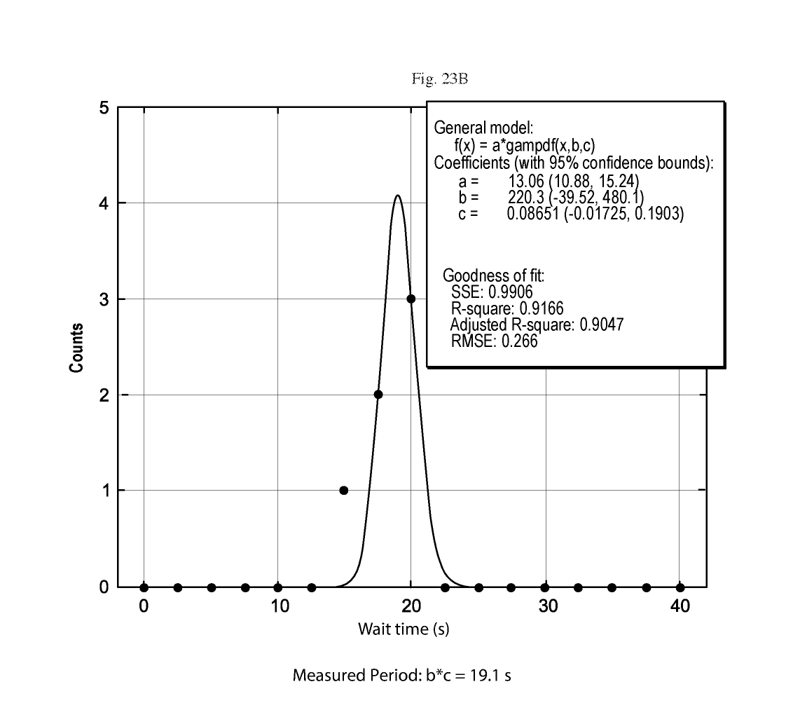

FIGS. 22A and 22B show a Monte Carlo simulation of polymerization from a 50-nucleotide circular template, demonstrating control of the period of fluorescent signal generation and precise determination of the period (+/−10% error).

### Example 11

FIGS. 23A and 23B show experimental total internal reflection fluorescence (TIRF) microscopy data demonstrating the generation of a periodic fluorescent signal (i.e., repeated pulses whose start times are separated by a well-defined delay) from a 179-nucleotide circular template, using a single-color fluorescent probe. The period was determined by measuring the time intervals in between the appearance of consecutive fluorescent signals.

### Example 12

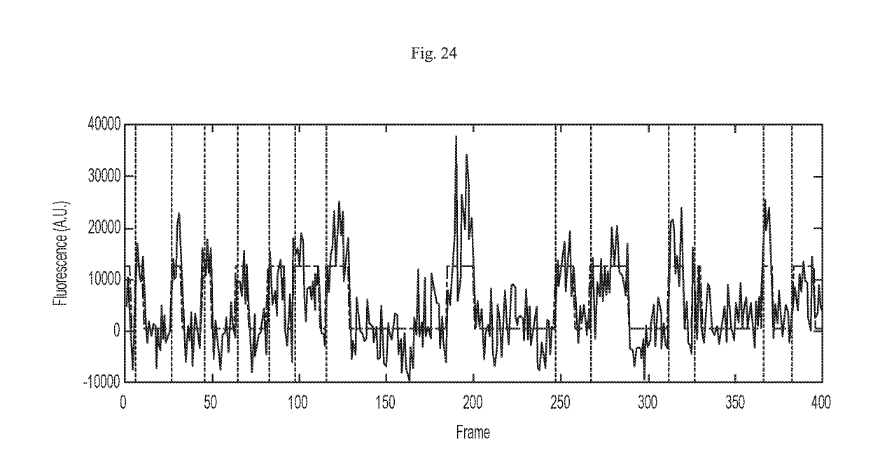

FIG. 24 shows that while periodic signaling is observed, there are also longer dark periods that may result from stalling of the polymerase. For instance, GC-rich DNA sequences are known to cause stalling of phi29 DNA polymerase. Modifying template sequences, reaction conditions, and polymerases can alter performance. Recording signal over several periods permits improved accuracy through averaging.

### Example 13

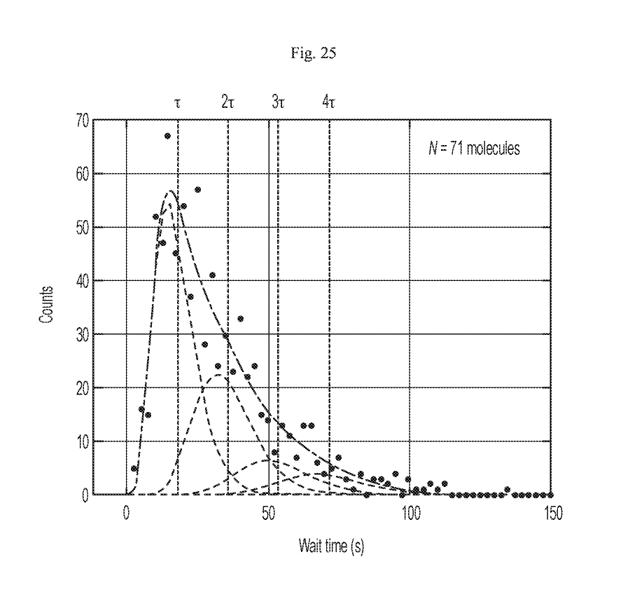

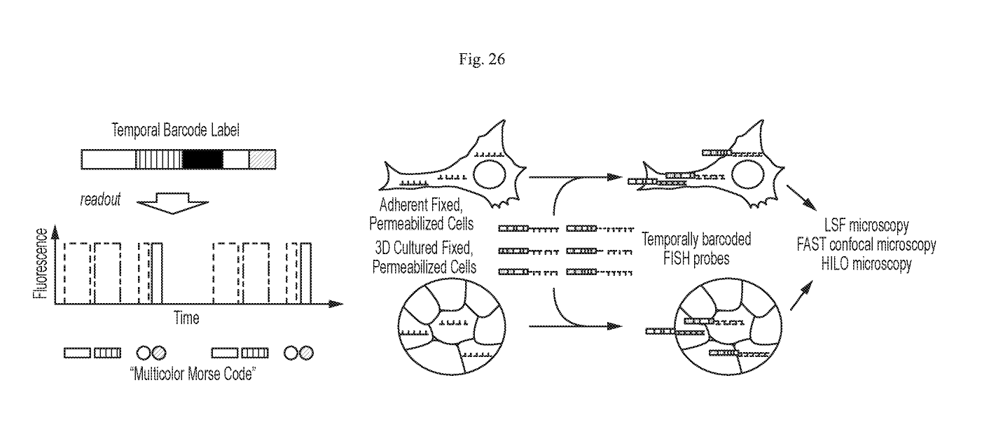

FIG. 25 shows a comprehensive histogram of wait times between consecutive fluorescent signaling events for a 179-nucleotide template. The maximum probability is at ˜19 s, with a significant tail extending out to ˜100 s. This tail may be the result of probe binding events that are not observed (e.g., due to bleaching of the fluorescent probe), resulting in wait times that are 2, 3, or 4 times longer than the expected length. Alternatively, the tail may be due to occasional stalling of the polymerase. In either case, averaging over multiple cycles improves the accuracy.

### Example 14

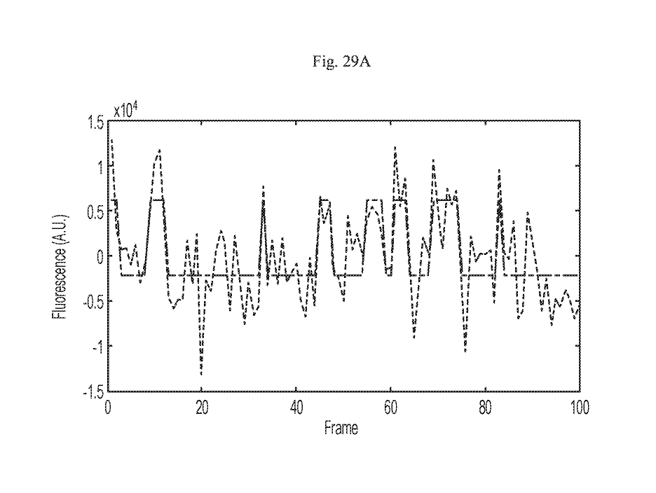

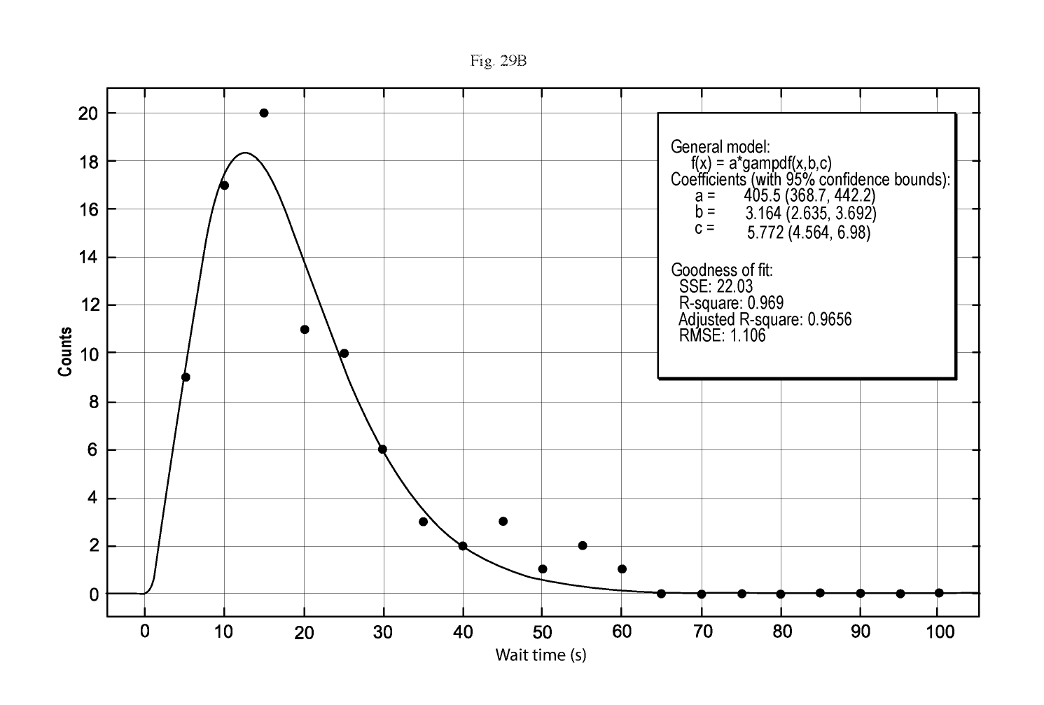

A catenane with an 84 nucleotide leash and a 179 nucleotide template was used. DNA replication was achieved by phi29 polymerase. Due to the direction of DNA replication, the toehold on the imager-quencher beacon was located on the 5′ end. Therefore, the fluorophore, Cy5 was available at the 3′-end. Iowa Black RQ was used as a quencher. The system is agnostic to the choice of fluorophore-quencher pairs. A generic imager-quencher pair is shown in FIG. 28A. N represents A, C, G, T, while K is the appropriate complement. Typically, a 5 μM concentration of imager strands in solution was used. A series dilution assay (imager:quencher ratios) was used to determine the ratio that would yield the lowest background and determined that 1:4 would be sufficient for minimal background (see FIG. 28B).

Catenanes were immobilized on a DDS passivated glass surface, using a biotin-streptavidin sandwich. Total Internal Reflection Fluorescence (TIRF) microscopy was performed to visualize the fluorescently labeled catenanes. TIRF videos were collected under 63× objective. The fluorescent blinking pattern observed and the wait times between individual pulses, shown in FIGS. 29A and 29B, suggest a reproducible cyclic behavior of the system. FIG. 29A shows the Monte Carlo fit to the data, which was used to estimate the wait times, shown in FIG. 29B.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of and “consisting essentially of shall be closed or semi-closed transitional phrases, respectively, as set forth in the United States Patent Office Manual of Patent Examining Procedures, Section 2111.03.

What is claimed is:

1. A kinetically encoded imaging system, comprising:
(a) an unpaired initiator nucleic acid comprising a 3′ nucleotide subdomain and a 5′ nucleotide subdomain;
(b) a template probe comprising (i) an unpaired 5′ toehold domain, (ii) a hairpin stem domain formed by base pairing between nucleotides located in a 5′ subdomain of the probe and nucleotides located in a 3′ subdomain of the probe, and a hairpin loop domain; and
(c) a primer.

2. The system of claim 1, wherein the primer is linked to a detectable molecule.

3. The system of claim 1, wherein the template probe is linked to a detectable molecule.

4. The system of claim 1, wherein the 3′ nucleotide subdomain of the initiator of (a) is complementary to and binds to the unpaired toehold domain of the probe of (b), and the 5′ nucleotide subdomain of the initiator of (a) is complementary to and binds to the 5′ subdomain of the probe of (b).

5. The system of claim 1, wherein the primer is complementary to and binds to the 3′ subdomain of the probe of (b).

6. The system of claim 1, wherein the template probe further comprises 3′ phosphate (PO₄²⁻) group.

7. The system of claim 1 further comprising a DNA polymerase.

8. The system of claim 7, wherein the DNA polymerase has strand displacement activity.

9. The system of claim 8, wherein the DNA polymerase is phi29.

10. The system of claim 8, wherein the DNA polymerase is Bst DNA polymerase, large fragment.

11. The system of claim 1, wherein the initiator nucleic acid has a length of 15-50 nucleotides.

12. The system of claim 11, wherein the initiator nucleic acid has a length of 20-30 nucleotides.

13. The system of claim 11, wherein the 3′ nucleotide subdomain of the initiator nucleic acid has a length of 5-15 nucleotides.

14. The system of claim 11, wherein the 5′ nucleotide subdomain of the initiator nucleic acid has a length of 10-20 nucleotides.

15. The system of claim 1, wherein the template probe has a length of 30-200 nucleotides.

16. The system of claim 15, wherein the template probe has a length of 30-50 nucleotides.

17. The system of claim 1,wherein the toehold domain has a length of 2-15 nucleotides.

18. The system of claim 1,wherein the hairpin stem domain has a length of 10-20 nucleotides.

19. The system of claim 1, wherein the hairpin loop domain has a length of 4-100 nucleotides.

20. The system of claim 19, wherein the hairpin loop domain has a length of 4-20 nucleotides.

21. The system of claim 1, wherein the primer has length of 10-20 nucleotides.

22. A kinetically encoded imaging method, comprising:
combining in reaction buffer
(a) an unpaired initiator nucleic acid comprising a 3′ nucleotide subdomain and a 5′ nucleotide subdomain, wherein the initiator nucleic acid is associated with a target of interest,
(b) a hairpin template probe comprising (i) an unpaired 5′ toehold domain, (ii) a hairpin stem domain formed by base pairing between nucleotides located in a 5′ subdomain of the probe and nucleotides located in a 3′ subdomain of the probe, and a hairpin loop domain,
(c) a primer linked to a detectable molecule,
(d) a DNA polymerase, and
(e) dNTPs, thereby forming a reaction mixture; and
incubating the reaction mixture under conditions that result in DNA polymerization.

23. The method of claim 22, wherein the 3′ nucleotide subdomain of the initiator of (a) is complementary to and binds to the unpaired toehold domain of the probe of (b), and the 5′ nucleotide subdomain of the initiator of (a) is complementary to and binds to the 5′ subdomain of the probe of (b).

24. The method of claim 22, wherein the primer is complementary to and binds to the 3′ subdomain of the probe of (b).

25. The method of claim 22 further comprising imaging the reaction mixture during the incubation step and identifying periods of time during which there is an increase in a level of fluorescence relative to a start time control level of fluorescence, thereby identifying dwell times.

26. The method of claim 25 further comprising identifying the presence or absence of a target of interest based on the dwell times.

27. The method of claim 22, wherein the initiator nucleic acid has a length of 15-50 nucleotides.

28. The method of claim 27, wherein the initiator nucleic acid has a length of 20-30 nucleotides

29. The method of claim 27, wherein the 3′ nucleotide subdomain of the initiator nucleic acid has a length of 5-15 nucleotides.

30. The method of claim 27, wherein the 5′ nucleotide subdomain of the initiator nucleic acid has a length of 10-20 nucleotides.

31. The method of claim 22, wherein the template probe has a length of 30-200 nucleotides.

32. The method of claim 31, wherein the template probe has a length of 30-50 nucleotides.

33. The method of claim 22,wherein the toehold domain has a length of 5-15 nucleotides.

34. The method of claim 22,wherein the hairpin stem domain has a length of 10-20 nucleotides.

35. The method of claim 22, wherein the hairpin loop domain has a length of 4-100 nucleotides.

36. The method of claim 35, wherein the hairpin loop domain has a length of 4-20 nucleotides.

37. The method of claim 22, wherein the primer has length of 10-20 nucleotides.

38. The method of claim 22, wherein the template probe further comprises 3′ phosphate (PO₄²⁻) group.

39. The method of claim 22, wherein the DNA polymerase has strand displacement activity.

40. The method of claim 39, wherein the DNA polymerase is phi29.

41. The method of claim 39, wherein the DNA polymerase is Bst DNA polymerase, large fragment.

42. The method of claim 22, wherein the detectable molecule is a fluorophore.

43. The method of claim 22, wherein dNTPs are present at a concentration of 2.5 μM to 10 mM.

44. The method of claim 43, wherein dNTPs are present at a concentration of 100 μM.

45. A nucleic acid molecule comprising a 5′ paired domain, an internal unpaired domain, and a 3′ paired domain linked to a detectable molecule.

46. A kinetically encoded imaging system, comprising:
(a) a target nucleic acid;
(b) a 5′-phosphorylated nucleic acid probe linked to a 3′ detectable molecule; and
(c) a 5′-phosphate-specific exonuclease.

47. The system of claim 46, wherein the probe is complementary to and binds to the target.

48. A kinetically encoded imaging method, comprising:
combining in reaction buffer
(a) a target nucleic acid,
(b) a 5′-phosphorylated nucleic acid probe linked to a 3′ detectable molecule, and
(c) a 5′-phosphate-specific exonuclease; and
incubating the reaction mixture under conditions that result in exonuclease-mediated degradation of the probe.

49. A kinetically encoded imaging system, comprising:
(a) an unpaired initiator nucleic acid; and
(b) a first hairpin probe, a second hairpin probe, a third hairpin probe and a fourth hairpin probe, each hairpin probe comprising (i) an unpaired 5′ toehold domain, (ii) a hairpin stem domain formed by intramolecular base pairing between nucleotides located in a 5′ subdomain of the probe and nucleotides located in a 3′ subdomain of the probe, and a hairpin loop domain located between the 5′ subdomain and the 3′ subdomain, wherein the first hairpin probe is linked to a detectable molecule.

50. The system of claim 49, wherein
(i) the toehold domain and the 5′ subdomain of the first probe are complementary to and bind to the initiator nucleic acid;
(ii) the toehold domain and the 5′ subdomain of the second probe are complementary to and bind to the hairpin stem domain and the 3′ subdomain of the first probe bound to the initiator sequence;
(iii) the toehold domain and the 5′ subdomain of the third probe are complementary to and bind to the hairpin stem domain and the 3′ subdomain of the second probe bound to the first probe; and
(iv) the toehold domain and the 5′ subdomain of the fourth probe are complementary to and bind to the hairpin stem domain and the 3′ subdomain of the third probe bound to the second probe, and wherein the hairpin loop and 3′ subdomain of the fourth probe are complementary to and bind to the toehold domain and the 5′ subdomain of the first probe.

51. A kinetically encoded imaging method, comprising:
combining in reaction buffer
(a) an unpaired initiator nucleic acid, and
(b) a first hairpin probe, a second hairpin probe, a third hairpin probe and a fourth hairpin probe, each hairpin probe comprising (i) an unpaired 5′ toehold domain, (ii) a hairpin stem domain formed by intramolecular base pairing between nucleotides located in a 5′ subdomain of the probe and nucleotides located in a 3′ subdomain of the probe, and a hairpin loop domain located between the 5′ subdomain and the 3′ subdomain, wherein the first hairpin probe is linked to a detectable molecule; and
incubating the reaction mixture under conditions that result in DNA hybridization.

52. A composition, comprising:
(a) a circular nucleic acid template comprising a primer binding sequence and interlocked with a circular nucleic acid leash;
(b) a nucleic acid primer comprising a sequence complementary to the primer binding sequence; and
(c) a labeled nucleic acid imager strand.

53. The composition of claim 52, wherein the template comprises deoxyribonucleic acid (DNA).

54. The composition of claim 52, wherein the leash is a circular nucleic acid leash strand.

55. The composition of claim 52, wherein the template has a length of 50-1000 nucleotides.

56. The composition of claim 55, wherein the template has a length of 50-500 nucleotides.

57. The composition of claim 52, wherein the leash has a length of 50-1000 nucleotides.

58. The composition of claim 52, wherein the primer comprises DNA or RNA.

59. The composition of claim 52, wherein the primer has a length of 5-50 nucleotides.

60. The composition of claim 52, wherein the imager strand comprises DNA.

61. The composition of claim 52, wherein the imager strand has a length of 5-50 nucleotides.

62. The composition of claim 52, wherein the imager strand comprises a sequence complementary to sequence encoded by the primer binding sequence.

63. The composition of claim 52, wherein the imager strand comprises a sequence complementary to a sequence encoded by the template, other than the reverse complement of the primer binding sequence.

64. The composition of claim 52, wherein the label is a fluorescent label.

65. The composition of claim 52, wherein the imager strand is bound to a quencher strand comprising a quencher molecule and wherein the quencher strand is shorter than the imager strand.

66. The composition of claim 52, wherein the fluorescent label is located at the 3′ end of the imager strand and the quencher molecule is located at the 5′ end of the quencher strand.

67. The composition of claim 52, wherein the composition further comprises an endonuclease.

68. The composition of claim 52 further comprising a polymerase selected from a DNA polymerase, a RNA polymerase and reverse transcriptase.

69. The composition of claim 68, wherein the polymerase has strand displacement activity.

70. The composition of claim 69, wherein the polymerase is a DNA polymerase.

71. The composition of claim 70, wherein the DNA polymerase is phi29 or Bst DNA polymerase, large fragment.

72. The composition of claim 52 further comprising at least one other labeled imager strand.

73. The composition of claim 72, wherein each of the other labeled imager strands comprises a sequence complementary to a distinct sequence encoded by the template.

74. The composition of claim 73, wherein the distinct sequences encoded by the template are separated from each by at least 10 nucleotides.

75. The composition of claim 72, wherein each of the imager strands comprises a spectrally-distinct fluorophore.

76. The composition of claim 52, wherein the circular nucleic acid leash is bound to an affinity tag that binds to a target of interest.

77. The composition of claim 76, wherein the target of interest is a protein or a nucleic acid.

78. A method, comprising:
combining in reaction buffer
(a) a circular nucleic acid template comprising a primer binding sequence and interlocked with a circular nucleic acid leash,
(b) a nucleic acid primer comprising a sequence complementary to the primer binding sequence,
(c) a labeled nucleic acid imager strand,
(d) a polymerase, and
(e) dNTPs or NTPs, thereby forming a reaction mixture; and
incubating the reaction mixture under conditions that result in nucleic acid polymerization and nucleic acid hybridization.

79. The method of claim 78, wherein the template comprises deoxyribonucleic acid (DNA).

80. The method of claim 78, wherein the template has a length of 50-1000 nucleotides.

81. The method of claim 80, wherein the template has a length of 50-500 nucleotides.

82. The method of claim 78, wherein the leash has a length of 50-1000 nucleotides.

83. The method of claim 78, wherein the primer comprises DNA.

84. The method of claim 78, wherein the primer has a length of 5-50 nucleotides.

85. The method of claim 78, wherein the imager strand comprises DNA.

86. The method of claim 78, wherein the imager strand has a length of 5-50 nucleotides.

87. The method of claim 78, wherein the imager strand comprises a sequence complementary to a distinct sequence encoded by the primer binding sequence.

88. The method of claim 78, wherein the imager strand comprises a sequence complementary to a distinct sequence encoded by the template, other than the reverse complement of the primer binding sequence.

89. The method of claim 78, wherein the label is a fluorescent label.

90. The method of claim 78, wherein the imager strand is bound to a quencher strand comprising a quencher molecule and wherein the quencher strand is shorter than the imager strand.

91. The method of claim 78, wherein the fluorescent label is located at the 3′ end of the imager strand and the quencher molecule is located at the 5′ end of the quencher strand.

92. The method of claim 78, wherein the composition further comprises an endonuclease.

93. The method of claim 78 wherein the polymerase is selected from a DNA polymerase, a RNB polymerase and reverse transcriptase.

94. The method of claim 93, wherein the polymerase has strand displacement activity.

95. The method of claim 94, wherein the polymerase is a DNA polymerase.

96. The method of claim 95, wherein the DNA polymerase is phi29 or Bst DNA polymerase, large fragment.

97. The method of claim 78 further comprising at least one other labeled imager strand.

98. The method of claim 97, wherein each of the other labeled imager strands comprises a sequence complementary to a distinct sequence encoded by the template.

99. The method of claim 98, wherein the distinct sequences encoded by the template are separated from each by at least 10 nucleotides.

100. The method of claim 97, wherein each of the imager strands comprises a spectrally-distinct fluorophore.

101. The method of claim 78, wherein the circular nucleic acid leash is bound to an affinity tag that binds to a target of interest.

102. The method of claim 101, wherein the target of interest is a protein or a nucleic acid.

103. The method of claim 78 further comprising imaging the reaction mixture during the incubation step and identifying periods of time during which there is an increase in a level of fluorescence relative to a start time control level of fluorescence, thereby identifying dwell times.

104. The method of claim 103 further comprising identifying the presence or absence of a target of interest based on the a pattern of fluorescence.

105. The method of claim 78, wherein dNTPs are present at a concentration of 2.5 μM to 10 mM.

106. The method of claim 105, wherein dNTPs are present at a concentration of 100 μM.

107. A composition, comprising:
(a) a circular nucleic acid template comprising a primer binding sequence and interlocked with a circular nucleic acid leash;
(b) a primer comprising a sequence complementary to the primer binding sequence; and
(c) a mixture of deoxynucleoside triphosphates (dNTPs) comprising subsets of dATPs, dTTPs, dCTPs and dGTPs, wherein dNTPs of at least one of the subsets comprise a label; or a mixture of nucleoside triphosphates (NTPs) comprising subsets of ATPs, TTPs, CTPs and GTPs, wherein NTPs of at least one of the subsets comprise a label.

108. The composition of claim 107, wherein the template comprises deoxyribonucleic acid (DNA).

109. The composition of claim 107, wherein the template has a length of 50-1000 nucleotides.

110. The composition of claim 109, wherein the template has a length of 50-500 nucleotides.

111. The composition of claim 107, wherein the leash has a length of 50-1000 nucleotides.

112. The composition of claim 107, wherein the primer comprises DNA.

113. The composition of claim 107, wherein the primer has a length of 5-50 nucleotides.

114. The composition of claim 107, wherein the label is a fluorescent label.

115. The composition of claim 107, wherein the imager strand is bound to a quencher strand comprising a quencher molecule and wherein the quencher strand is shorter than the imager strand.

116. The composition of claim 107, wherein the fluorescent label is located at the 3′ end of the imager strand and the quencher molecule is located at the 5′ end of the quencher strand.

117. The composition of claim 107, wherein the composition further comprises an endonuclease.

118. The composition of claim 107 further comprising a polymerase selected from a DNA polymerase, a RNA polymerase and reverse transcriptase.

119. The composition of claim 118, wherein the polymerase has strand displacement activity.

120. The composition of claim 119, wherein the polymerase is a DNA polymerase.

121. The composition of claim 120, wherein the DNA polymerase is phi29 or Bst DNA polymerase, large fragment.

122. The composition of claim 107, wherein the circular nucleic acid leash is bound to an affinity tag that binds to a target of interest.

123. The composition of claim 122, wherein the target of interest is a protein or a nucleic acid.

124. A method, comprising:
combining in reaction buffer
(a) a circular nucleic acid template comprising a primer binding sequence and interlocked with a circular nucleic acid leash,
(b) a primer comprising a sequence complementary to the primer binding sequence,
(c) a mixture of dNTPs comprising subsets of dATPs, dTTPs, dCTPs and dGTPs, wherein dNTPs of at least one of the subsets comprise a label, or mixture of NTPs comprising subsets of ATPs, TTPs, CTPs and GTPs, wherein NTPs of at least one of the subsets comprise a label, and
(d) a polymerase; and
incubating the reaction mixture under conditions that result in nucleic acid polymerization and nucleic acid hybridization.

125. The method of claim 124, wherein the template comprises deoxyribonucleic acid (DNA).

126. The method of claim 124, wherein the template has a length of 50-1000 nucleotides.

127. The method of claim 126, wherein the template has a length of 50-500 nucleotides.

128. The method of claim 124, wherein the leash has a length of 50-1000 nucleotides.

129. The method of claim 124, wherein the primer comprises DNA.

130. The method of claim 124, wherein the primer has a length of 5-50 nucleotides.

131. The method of claim 124, wherein the label is a fluorescent label.

132. The method of claim 124, wherein the imager strand is bound to a quencher strand comprising a quencher molecule and wherein the quencher strand is shorter than the imager strand.

133. The method of claim 124, wherein the fluorescent label is located at the 3′ end of the imager strand and the quencher molecule is located at the 5′ end of the quencher strand.

134. The method of claim 124, wherein the composition further comprises an endonuclease.

135. The method of claim 124 wherein the polymerase is selected from a DNA polymerase, a RNA polymerase and reverse transcriptase.

136. The method of claim 135, wherein the polymerase has strand displacement activity.

137. The method of claim 136, wherein the polymerase is a DNA polymerase.

138. The method of claim 137, wherein the DNA polymerase is phi29 or Bst DNA polymerase, large fragment.

139. The method of claim 124, wherein the circular nucleic acid leash is bound to an affinity tag that binds to a target of interest.

140. The method of claim 139, wherein the target of interest is a protein or a nucleic acid.

141. The method of claim 124 further comprising imaging the reaction mixture during the incubation step and identifying periods of time during which there is an increase in a level of fluorescence relative to a start time control level of fluorescence, thereby identifying dwell times.

142. The method of claim 141 further comprising identifying the presence or absence of a target of interest based on the a pattern of fluorescence.

143. The method of claim 124, wherein dNTPs are present at a concentration of 2.5 μM to 10 mM.

144. The method of claim 143, wherein dNTPs are present at a concentration of 100 μM.

145. A composition, comprising:
(a) a circular nucleic acid template comprising a primer binding sequence and interlocked with a circular nucleic acid leash;
(b) a nucleic acid primer comprising a sequence complementary to the primer binding sequence; and
(c) a labeled nucleic acid imager strand bound to a quencher strand comprising a quencher molecule, wherein the quencher strand is shorter than the imager strand.

146. The composition of claim 145, wherein the fluorescent label is located at the 3′ end of the imager strand and the quencher molecule is located at the 5′ end of the quencher strand.

147. The composition of claim 145, wherein the composition further comprises an endonuclease.

148. A method, comprising:
combining in reaction buffer
(a) a circular nucleic acid template comprising a primer binding sequence and interlocked with a circular nucleic acid leash,
(b) a nucleic acid primer comprising a sequence complementary to the primer binding sequence,
(c) a labeled nucleic acid imager strand bound to a quencher strand comprising a quencher molecule, wherein the quencher strand is shorter than the imager strand,
(d) a polymerase, and
(e) dNTPs or NTPs, thereby forming a reaction mixture; and
incubating the reaction mixture under conditions that result in nucleic acid polymerization and nucleic acid hybridization.

149. The method of claim 148, wherein the fluorescent label is located at the 3′ end of the imager strand and the quencher molecule is located at the 5′ end of the quencher strand.

150. The method of claim 148, wherein the reaction buffer further comprises an endonuclease.
